(19) United States
(12) Patent Application Publication
(10) Pub. No.: US 2026/0243756 A1
(43) Pub. Date: Aug. 20, 2026

(54) 3-PHOSPHOINOSITIDE BIOSENSORS

(71) Applicant: The Regents of the University of California, Oakland, CA (US)

(72) Inventors: Jin Zhang, San Diego, CA (US); Ayse Z. Sahan, Sunnyvale, CA (US); Mingyuan Chen, San Diego, CA (US)

(21) Appl. No.: 19/540,146

(22) Filed: Feb. 13, 2026

Related U.S. Application Data
(60) Provisional application No. 63/758,570, filed on Feb. 14, 2025.

(51) Int. Cl. G01N 33/50; C07K 14/705

(52) U.S. Cl. CPC G01N 33/5035; C07K 14/705; G01N 33/5041

(57) ABSTRACT

This document relates to engineered 3-phosphoinositide (3-PI) biosensor polypeptides including a lipid binding domain and a pseudoligand configured for detecting a target 3-PI in a cell via a detectable signal. In some cases, such biosensor polypeptides are useful for detecting the cellular localization of a target lipid (e.g., a target 3-PI) and/or for assessing Phosphoinositide 3-Kinase (PI3K)/Akt signaling pathway activity in a cell.

### CROSS-REFERENCE TO RELATED APPLICATIONS

This application claims the benefit of U.S. Provisional Application Ser. No. 63/758,570, filed on Feb. 14, 2025. The entire contents of the foregoing are incorporated herein by reference.

### FEDERALLY SPONSORED RESEARCH OR DEVELOPMENT

This invention was made with government support under CA197622, DE030497, GM007752, and DE032886 awarded by the National Institutes of Health. The government has certain rights in the invention.

### SEQUENCE LISTING

This application contains a Sequence Listing that has been submitted electronically as an XML file named 15670-0450001_SL_ST26.xml. The XML file, created on Feb. 10, 2026, is 60,851 bytes in size. The material in the XML file is hereby incorporated by reference in its entirety

### TECHNICAL FIELD

This document relates to engineered 3-phosphoinositide (3-PI) biosensor polypeptides. For example, this document provides biosensor polypeptides that include a lipid binding domain and a pseudoligand configured for detecting a target lipid (e.g., a target 3-PI) in a cell via a detectable signal (e.g., a fluorescent signal). In some aspects, such biosensor polypeptides are useful for detecting the cellular localization of a target lipid (e.g., a target 3-PI) and/or for assessing Phosphoinositide 3-Kinase (PI3K)/Akt signaling pathway activity in a cell.

### BACKGROUND

3-PIs, phosphatidylinositol (3,4) bisphosphate (PI(3,4)P₂) and phosphatidylinositol (3,4,5) trisphosphate (PIP₃), are important lipid second messengers in the PI3K/Akt signaling pathway, which is crucial to cell growth and frequently dysregulated in cancer. Emerging evidence suggests these lipid second messengers may be present in membranes beyond the plasma membrane, yet their spatial regulation within other membrane compartments is not well understood.

### SUMMARY

Provided herein are engineered 3-PI biosensor polypeptides that leverage a pseudoligand-based design. The biosensor polypeptides provided herein incorporate a lipid binding domain that can bind 3-PIs and a pseudoligand that competes with 3-PIs for binding to the lipid binding domain. Upon a 3-PI outcompeting the pseudoligand for binding to the lipid binding domain, the biosensor polypeptide undergoes a detectable conformational change. Such biosensor polypeptides allow for assessment of cellular spatial regulation and dynamics of 3-PIs with enhanced phospholipid targetability and potential for affinity tuning.

Accordingly, aspects of the present disclosure provide biosensor polypeptides comprising (a) a first linker; (b) a lipid binding domain comprising the amino acid sequence set forth in SEQ ID NO: 1; (c) a second linker; (d) a pseudoligand comprising the amino acid sequence set forth in SEQ ID NO:9; and (e) a third linker.

In some embodiments the biosensor polypeptide comprises, in order from amino terminus (N-terminus) to carboxy terminus (C-terminus), (a) the first linker; (b) the lipid binding domain comprising the amino acid sequence set forth in SEQ ID NO:1; (c) the second linker; (d) the pseudoligand comprising the amino acid sequence set forth in SEQ ID NO:9; and (e) the third linker.

In some embodiments, the N-terminus of the lipid binding domain is attached to the C-terminus of the first linker domain, the N-terminus of the second linker domain is attached the C-terminus of the lipid binding domain, the N-terminus of the pseudoligand is attached to the C-terminus of the second linker domain, and the N-terminus of the third linker is attached to the C-terminus of the pseudoligand.

In some embodiments, the lipid binding domain is capable of binding phosphatidylinositol(3,4,5)trisphosphate (PIP₃).

In some embodiments, the pseudoligand is capable of binding to the lipid binding domain.

In some embodiments, the biosensor polypeptide further comprises a donor and acceptor moiety pair including a donor moiety and an acceptor moiety. In some embodiments, the N-terminus of the first linker is attached to the C-terminus of the donor moiety, and the N-terminus of the acceptor moiety is attached to the C-terminus of the third linker. In some embodiments, the donor moiety and the acceptor moiety comprise a fluorescence resonance energy transfer (FRET) pair, a chemiluminescence resonance energy transfer (CRET) pair, or a bioluminescence resonance energy transfer (BRET) pair. In some embodiments, the donor moiety is a fluorophore donor, and the acceptor moiety is a fluorophore acceptor. In some embodiments, the fluorophore donor is a cerulean fluorescent polypeptide, a cyan fluorescent polypeptide, a blue fluorescent polypeptide, a green fluorescent polypeptide, a yellow fluorescent polypeptide, a red fluorescent polypeptide, a mT-Sapphire fluorescent polypeptide, a mTagBFP fluorescent polypeptide, a mAmertrine fluorescent polypeptide, a mTurquoise2 fluorescent polypeptide, a CyPet fluorescent polypeptide, or a LSSmOrange fluorescent polypeptide. In some embodiments, the fluorophore acceptor is a citrine fluorescent polypeptide, a cpVenus (VE172) fluorescent polypeptide, a tdTomato fluorescent polypeptide, a sfGFP fluorescent polypeptide, a mVenus fluorescent polypeptide, an EYFP fluorescent polypeptide, a mNeonGreen fluorescent polypeptide, a sREACh1 fluorescent polypeptide, a YPet fluorescent polypeptide, a mKate2 fluorescent polypeptide, a green fluorescent polypeptide, a yellow fluorescent polypeptide, a red fluorescent polypeptide, an orange fluorescent polypeptide, or a far-red fluorescent polypeptide. In some embodiments, the donor moiety is a chemiluminescence donor and the acceptor moiety is a chemiluminescence acceptor.

In some embodiments, the first linker comprises the amino acid sequence set forth in SEQ ID NO:10.

In some embodiments, the second linker comprises the amino acid sequence set forth in SEQ ID NO:11.

In some embodiments, the third linker comprises the amino acid sequence set forth in SEQ ID NO:12.

In some embodiments, the biosensor polypeptide further comprises a nucleus targeting sequence, a Golgi apparatus targeting sequence, a lysosome targeting sequence, a plasma membrane targeting sequence, an endosome targeting sequence, an endoplasmic reticulum targeting sequence, and a mitochondria targeting sequence.

In some embodiments, the targeting sequence comprises an amino acid sequence set forth in any one of SEQ ID NOs:43-54.

Aspects of the present disclosure provide biosensor polypeptides comprising (a) a first linker; (b) a lipid binding domain comprising the amino acid sequence set forth in SEQ ID NO:2; (c) a second linker; (d) a pseudoligand comprising the amino acid sequence set forth in SEQ ID NO:9; and (e) a third linker.

In some embodiments, the biosensor polypeptide comprises, in order from amino terminus (N-terminus) to carboxy terminus (C-terminus), (a) the first linker; (b) the lipid binding domain comprising the amino acid sequence set forth in SEQ ID NO:2; (c) the second linker; (d) the pseudoligand comprising the amino acid sequence set forth in SEQ ID NO:9; and (e) the third linker.

In some embodiments, the N-terminus of the lipid binding domain is attached to the C-terminus of the first linker domain, the N-terminus of the second linker domain is attached the C-terminus of the lipid binding domain, the N-terminus of the pseudoligand is attached to the C-terminus of the second linker domain, and the N-terminus of the third linker is attached to the C-terminus of the pseudoligand.

In some embodiments, the lipid binding domain is capable of binding phosphatidylinositol(3,4)bisphosphate (PI(3,4)P₂).

In some embodiments, the pseudoligand is capable of binding to the lipid binding domain.

In some embodiments, the biosensor polypeptide further comprises a donor and acceptor moiety pair including a donor moiety and an acceptor moiety. In some embodiments, the N-terminus of the first linker is attached to the C-terminus of the donor moiety, and the N-terminus of the acceptor moiety is attached to the C-terminus of the third linker. In some embodiments, the donor moiety and the acceptor moiety comprise a fluorescence resonance energy transfer (FRET) pair or a chemiluminescence resonance energy transfer (CRET) pair. In some embodiments, the donor moiety is a fluorophore donor, and the acceptor moiety is a fluorophore acceptor. In some embodiments, the fluorophore donor is a cerulean fluorescent polypeptide, a cyan fluorescent polypeptide, a blue fluorescent polypeptide, a green fluorescent polypeptide, a yellow fluorescent polypeptide, a red fluorescent polypeptide, a mT-Sapphire fluorescent polypeptide, a mTagBFP fluorescent polypeptide, a mAmertrine fluorescent polypeptide, a mTurquoise2 fluorescent polypeptide, a CyPet fluorescent polypeptide, or a LSSmOrange fluorescent polypeptide. In some embodiments, the fluorophore acceptor is a citrine fluorescent polypeptide, a cpVenus (VE172) fluorescent polypeptide, a tdTomato fluorescent polypeptide, a sfGFP fluorescent polypeptide, a mVenus fluorescent polypeptide, an EYFP fluorescent polypeptide, a mNeonGreen fluorescent polypeptide, a sREACh1 fluorescent polypeptide, a YPet fluorescent polypeptide, a mKate2 fluorescent polypeptide, a green fluorescent polypeptide, a yellow fluorescent polypeptide, a red fluorescent polypeptide, an orange fluorescent polypeptide, or a far-red fluorescent polypeptide. In some embodiments, the donor moiety is a chemiluminescence donor and the acceptor moiety is a chemiluminescence acceptor.

In some embodiments, the first linker comprises the amino acid sequence set forth in SEQ ID NO:10.

In some embodiments, the second linker comprises the amino acid sequence set forth in SEQ ID NO:11.

In some embodiments, the third linker comprises the amino acid sequence set forth in SEQ ID NO:12.

In some embodiments, the biosensor further comprises a nucleus targeting sequence, a Golgi apparatus targeting sequence, a lysosome targeting sequence, a plasma membrane targeting sequence, an endosome targeting sequence, an endoplasmic reticulum targeting sequence, or a mitochondria targeting sequence. In some embodiments, the targeting sequence comprises an amino acid sequence set forth in any one of SEQ ID NOs:43-54.

Aspects of the present disclosure provide a nucleic acid sequence encoding a biosensor polypeptide provided herein.

Aspects of the present disclosure provide a vector comprising a nucleic acid sequence encoding a biosensor polypeptide provided herein.

In some embodiments, the vector is an expression plasmid.

In some embodiments, the vector is a viral vector. In some embodiments, the viral vector is a vector based on an adenovirus, a vector based on an adeno-associated virus (AAV), a vector based on a retrovirus, a vector based on an lentivirus, or a vector based on a baculovirus.

Aspects of the present disclosure provide a method for detecting the cellular localization of a polypeptide in a cell, wherein the method comprises (a) contacting the cell with a biosensor polypeptide provided herein, a nucleic acid encoding a biosensor polypeptide provided herein, or a vector comprising a nucleic acid encoding a biosensor polypeptide provided herein; and (b) detecting a signal from the donor and acceptor moiety pair.

In some embodiments, the method comprises detecting a change in the signal upon binding of a lipid to the lipid binding domain.

In some embodiments, the signal from the donor and acceptor moiety pair is the acceptor moiety emission to donor moiety emission ratio.

Aspects of the present disclosure provide a method for detecting phosphoinositide 3-kinase (PI3K) pathway signaling in a cell, wherein the method comprises (a) contacting the cell with a biosensor polypeptide provided herein, a nucleic acid encoding a biosensor polypeptide provided herein, or a vector comprising a nucleic acid encoding a biosensor polypeptide provided herein; and (b) detecting a signal from the donor and acceptor moiety pair.

In some embodiments, the method comprises detecting a change in the signal upon binding of a lipid to the lipid binding domain.

In some embodiments, the signal from the donor and acceptor moiety pair is the acceptor moiety emission to donor moiety emission ratio.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D. Development and characterization of InPGrp. FIG. 1A) Domain structure (upper) and schematic showing the modulation of FRET between cyan and yellow fluorescent protein by a molecular switch consisting of Grp1 PH domain and a pseudoligand. The Grp1 PH domain and pseudoligand structures are depicted as predicted by AlphaFold. FIG. 1B) Changes in the normalized emission ratio (yellow/cyan) of InPGrp-Kras (n=26, maximum response or Rₘₐₓ=8.9 0.6%, n=26) and InPGrp-Kras K273A mutant (n=19, Rₘₐₓ=0.5±0.06%, n=20) in serum starved NIH3T3 cells stimulated with 50 ng/ml PDGF shown over time in time-course graph (left) and as maximum response values (right). Data collected from three independent experiments. Cell image shows plasma membrane localization of the InPGrp-Kras biosensor, scale bar=10 μm. Error bar represents mean±SEM. ****, P<0.0001, unpaired t test with Welch's correction. FIG. 1C) Domain structure of plasma membrane targeted lipid phosphatases and their targeted lipids: PTEN A4-Kras, INPP4B-Kras, and INPP5E-Kras, using CAAX targeting motif, mCherry-Kras was used as the control. FIG. 1D) Representative time-course curves from one individual experiment (left) showing changes in normalized emission ratio (yellow/cyan) of InPGrp-Kras, or InPGrp-Kras co-expressed with lipid phosphatases including PTEN-A4-Kras, INPP4B-Kras, INPP5E-Kras, and mCherry-Kras. Error bar represents mean±SEM. WT: n=5; PTENA4: n=5; INPP4B: n=4; INPP5E: n=4; mCherry: n=4. Bar graph showing maximum normalized emission ratio (Yellow/Cyan) for each cell for data collected across 3 independent experiments. Error bar represents mean±SEM. WT, Rₘₐₓ=8.9±0.6%, n=26; PTENA4-KRAS, Rₘₐₓ=0.6±0.4%, n=16; INPP5E-Kras, Rₘₐₓ=1.1±0.7%, n=15; INPP4B-Kras, Rₘₐₓ=8.4±0.6%, n=19; mCherry-Kras, Rₘₐₓ=7.9±0.6%, n=15; Data from three independent experiments each, WT data reproduced from FIG. 1B. ****, P<0.0001, Statistical significance tested by ordinary one-way ANOVA followed by Dunnett's multiple comparison test.

FIGS. 2A-2D. Development and characterization of InPTapp. FIG. 2A) Domain structure (upper) and schematic showing the modulation of FRET between cyan and yellow fluorescent protein by a molecular switch consisting of Tapp1 PH domain and a pseudoligand. The Tapp1 PH domain and pseudoligand structures are depicted as predicted by AlphaFold. FIG. 2B) Changes in the normalized emission ratio (yellow/cyan) of InPTapp-Kras (n=25, Rₘₐₓ=9.2±0.3%) and InPTapp-Kras R211L mutant (n=19, Rₘₐₓ=0.1±0.05%) following PDGF stimulation in serum starved NIH3T3 cells stimulated with 50 ng/ml PDGF. Data collected from three independent experiments. Cell image shows correct plasma membrane localization of the InPTapp-Kras biosensor. Scale bar=10 μm. Error bar represents mean±SEM. ****, P<0.0001, unpaired t test with Welch's correction. FIG. 2C) Domain structure of plasma membrane targeted lipid phosphatases and their targeted lipid: PTEN A4-Kras, INPP4B-Kras, and INPP5E-Kras, using CAAX targeting motif. mCherry-Kras was used as the control. FIG. 2D) Representative single cell curve of one individual experiment showing changes in normalized emission ratio (Yellow/Cyan) of InPTapp-Kras, or InPTapp-Kras co-expressed with lipid phosphatases including PTEN-A4-Kras, INPP4B-Kras, INPP5E-Kras, and mCherry-Kras. Error bar represents mean SEM, WT: n=4; PTENA4: n=5; INPP4B: n=3; INPP5E: n=4; mCherry: n=3. Bar graph showing maximum normalized emission ratio (yellow/cyan) for each cell for data collected across 3 independent experiments. WT, Rₘₐₓ=9.2 0.4%, n=25; PTENA4-Kras, Rₘₐₓ=0.3±0.4%, n=19; INPP4B-Kras, Rₘₐₓ=0.5±0.4%, n=16; INPP5E-Kras, Rₘₐₓ=9±0.6%, n=16; mCherry-Kras, Rₘₐₓ=8.4±0.5%, n=16. Data from three independent experiments each, WT data reproduced from FIG. 2B. ****, P<0.0001, ordinary one-way ANOVA followed by Dunnett's multiple comparison test.

FIGS. 3A-3E. PIP₃ and PI(3,4)P₂ accumulate at the lysosome. FIG. 3A) Domain structure of Lyso-InPGrp and Lyso InPTapp, and confocal images of Lyso-InPGrp (upper) or Lyso-InPTapp (lower) with LysoTracker Red (Sensor, middle; Lyso Tracker, left; merged, right). Scale bar=10 μm. FIG. 3B) Average changes in the normalized emission ratio (yellow/cyan) of Lyso-InPGrp (Rₘₐₓ=2.8±0.2%, n=30) and Lyso-InPTapp (Rₘₐₓ=1.3±0.1%, n=18) following PDGF stimulation in serum starved NIH3T3 cells stimulated with 50 ng/ml PDGF. Data collected from three independent experiments. Cell image shows correct plasma membrane localization of the InPTapp-Kras biosensor. Error bar represents mean±SEM. ****, P<0.0001, unpaired t test with Welch's correction. FIG. 3C) t_{1/2} comparing PIP₃ and PI(3,4)P₂ kinetics at the plasma membrane (left) and lysosomes (right). InPGrp, plasma membrane, t_{1/2}=1.732±0.111 mins, n=26, Lyso, t_{1/2}=2.074±0.085 mins, n=30; InPTapp, plasma membrane, t_{1/2}=3.311 0.411 mins, n=25, lysosome, t_{1/2}=2.430 0.153 mins, n=18. **, P=0.0014, *, P=0.0321, unpaired t test with Welch's correction. FIG. 3D) Response amplitude or maximum response, Rₘₐₓ, comparison between InPGrp (PIP₃) and InPTapp (PI(3,4)P₂) at the plasma membrane and at the lysosome. InPGrp, plasma membrane, Rₘₐₓ=8.9±0.6%, n=26, Lyso, Rₘₐₓ=2.8±0.2%, n=30; InPTapp, plasma membrane, Rₘₐₓ=9.2±0.3%, n=25, Lyso, Rₘₐₓ=1.3±0.1%, n=18. ns, not significant, P=0.7189, ***, P<0.001, unpaired t test with Welch's correction. FIG. 3E) SAM15 (sustainable activity metric at 15 mins) comparing PIP₃ and PI(3,4)P₂ at the plasma membrane (left) and lysosomes (right). InPGrp, plasma membrane, SAM15=0.9707±0.004, n=26, Lyso, SAM15=0.238+0.113, n=25; plasma membrane, SAM15=0.713±0.033, n=25, Lyso, SAM15=−0.398 0.198, n=18. ****, P<0.0001, **, P=0.0015, unpaired t test with Welch's correction. Metrics from panels C-E were analyzed based on data from three independent experiments and error bars show mean±SD.

FIGS. 4A-4B. Lysosomal PIP₃ and PI(3,4)P₂ accumulation is dependent on endocytosis. FIG. 4A) Average cell curves (left) of Lyso-InPGrp following PDGF stimulation in serum starved NIH3T3 cells (WT, Rₘₐₓ=2.8±0.3%, n=30, Dyngo-4a, Rₘₐₓ<0.01±0.2%, n=18; DN Dynamin, Rₘₐₓ<0.01±0.2%, n=18). Quantified response represented in a bar graph (right). Error bar shows mean±SEM. *, P=0.0431. ****, P<0.0001. Data from three independent experiments, WT data from FIG. 3B was reproduced. FIG. 4B) Average cell curves (left) of Lyso-InPTapp following PDGF stimulation in serum starved NIH13T3 cells (WT, Rₘₐₓ=1.3±0.2%, n=18; Dyngo-4a, Rₘₐₓ=0.1±0.08%, n=12; DN Dynamin, Rₘₐₓ=0.4±0.08%, n=20). Quantified response represented in a bar graph (right). Error bar shows mean SEM. **, P=0.0096. ***, P=0.0002. ****, P<0.0001. Data from three independent experiments, WT data from FIG. 3B was reproduced.

FIGS. 5A-5D. PIP₃ is generated at the plasma membrane and subsequently internalized to the lysosome. FIG. 5A) Diagram showing experimental design for identifying the path of PIP₃ to the lysosome. Plasma membrane targeted lipid phosphatases such as INPP5E-mCherry deplete PIP₃ at the plasma membrane, and thereby inhibit trafficking and accumulation of PIP₃ at lysosomes. FIG. 5B) Representative confocal images of serum starved NIH3T3 cells expressing mCherry-tagged phosphatases (top) with LysoTracker green (middle) or lysosome-targeted InPGrp (positive control, top) and with Lysotracker red (middle), scale bar=10 μm. FIG. 5C) Domain structure of plasma membrane targeted lipid phosphatases and their targeted lipid: PTEN A4-Kras, INPP4B-Kras, and INPP5E-Kras, using CAAX targeting motif mCherry-Kras was used as the control. FIG. 5D) Representative single cell curves of one individual experiment showing changes in normalized emission ratio (yellow/cyan) of Lyso-InPGrp or co-expressed with lipid phosphatases including PTENA4-Kras, INPP4B-Kras, INPP5E-Kras, and mCherry-Kras (WT, n=5; PTENA4, n=4; INPP4B, n=6; INPP5E, n=5; mCherry, n=4) Statistically analyzed Lyso-InPGrp response. WT, Rₘₐₓ=2.6±0.4%, n=30; PTENA4, Rₘₐₓ=0.1±0.1%, n=21; INPP4B, Rₘₐₓ=2.1±0.2%, n=21; INPP5E, Rₘₐₓ=0.1±0.1%, n=17; mCherry, Rₘₐₓ=3.1±0.3%, n=12. Data from three independent experiments each. Ordinary one-way ANOVA followed by Dunnett's multiple comparison test. **** P<0.0001.

FIGS. 6A-6D. PIP₃ accumulates at endosomes en route to lysosomes. FIG. 6A) Average cell curves (left) of PDGF stimulated Endo-InPGrp (WT, Rₘₐₓ=5.3±0.4%, n=36) or mutant Endo-InPGrp (mutant, Rₘₐₓ=1.0±0.5%, n=14) response in serum starved NIH3T3 cells. Quantified response represented in a bar graph (right). Error bar shows mean±SEM. WT n=36, mutant n=14, **, P<0.0001, unpaired t-test with Welch's correction. Data from three independent experiments. FIG. 6B) Diagram showing experimental design for identifying the path of PIP₃ to the lysosome. Endosome targeted lipid phosphatase INPP5E-mCherry depletes PIP₃ at the endosome, and thereby inhibits trafficking and accumulation of PIP₃ at lysosomes. FIG. 6C) Epifluorescence images of cells expressing Lyso-InPGrp (left) with INPP5E-2×FYVE (middle) separately or merged (right). Scale bar=20 μM. FIG. 6D) Average cell curves (left) of PDGF stimulated Lyso-InPGrp response in serum starved NIH3T3 cells with co-expression of INPP5E-2×FYVE or mCherry-2×FYVE. Quantified response represented in a bar graph (right). Error bar shows mean±SEM. mCherry, Rₘₐₓ=3.4±0.3%, n=13; INPP5E, Rₘₐₓ=2.0±0.3%, n=10, **, P<0.01, unpaired t-test with Welch's correction. Data from three independent experiments.

FIGS. 7A-7D. The predicted interactions between the pseudoligand and Grp1 PH domain reveal the conformational change underlying the FRET change of InPGrp. FIG. 7A) The resolved structure of PIP₃-bound Grp1 PH domain as reported elsewhere (20). PIP₃ is shown, the K273A mutation site in Grp1 which abolishes lipid binding is, and lipid binding residues are shown. FIG. 7B) The predicted interaction between the wild type Grp1 PH domain and the pseudoligand of the InPGrp biosensor is shown. The K273Amutation site in Grp1 which abolishes lipid binding is shown, lipid binding residues which interact with the pseudoligand are shown, and non-lipid binding residues which interact with the pseudoligand are shown. FIG. 7C) The predicted interaction between the wild type Grp1 PH domain and the pseudoligand of the InPGrp biosensor is shown when in the presence of PIP₃. The residues interacting with the pseudoligand which are non-lipid binding residues are shown. The pseudoligand is predicted to be competed out of the lipid binding pocket when PIP3 is present. FIG. 7D) The predicted interaction between the mutant K273A Grp1 PH domain and the pseudoligand of the InPGrp biosensor is shown. The K273A mutation is shown, lipid binding residues which interact with the pseudoligand are shown, and non-lipid binding residues which interact with the pseudoligand are shown.

FIGS. 8A-8C. Plasma membrane InPGrp response is dependent on PI3K and PIP₃. FIG. 8A) Average cell curves (left) of PDGF stimulated InPGrp-Kras response in serum starved NIH3T3 cells with or without (+/−) PIK-75 pretreatment. Quantified response represented in a bar graph (right). Error bar shows mean±SEM; −PIK75, Rₘₐₓ=4.6±0.8%, n=18; +PIK75, Rₘₐₓ<0.01±0.8%, n=15; ****, P<0.001, unpaired t-test with Welch's correction. Data from three independent experiments. FIG. 8B) InPGrp-Kras responses plotted against RFP fluorescence intensities in individual cells across three co-expressed lipid phosphatases: PTEN A4, INPP4B, INPP5E, and mCherry control by itself. X-axis shows RFP intensity at the plasma membrane; y-axis shows the maximal emission ratio of InPGrp-Kras at the plasma membrane. FIG. 8C) Lipid-protein interaction assay on a PIP strip using purified InPGrp shows specificity for InPGrp to PI(3,4,5)P₃ over other phosphoinositides. InPGrp protein was purified and incubated on the PIP strip. Bound InPGrp was detected by anti-6×Histag antibody and chemiluminescent detection reagents. For the positive control, InPGrp protein was blotted and dried directly on the PIP strip membrane.

FIGS. 9A-9D. The predicted interactions between the pseudoligand and Tapp1 PH domain reveal the conformational change underlying the FRET change of InPTapp. FIG. 9A) The resolved structure of citrate-bound Tapp1 PH domain, mimicking PI(3,4)P₂ binding, is shown as reported by Thomas et al (33). Citrate is shown, the R211L mutation site in Tapp1 which abolishes lipid binding is shown, and lipid binding residues are shown. FIG. 9B) The predicted interaction between the wild type Tapp1 PH domain and the pseudoligand of the InPTapp biosensor is shown. The R211L mutation site in Tapp1 which abolishes lipid binding is shown, lipid binding residues which interact with the pseudoligand are shown, and non-lipid binding residues which interact with the pseudoligand are shown. FIG. 9C) The predicted interaction between the wild type Tapp1 PH domain and the pseudoligand of the InPTapp biosensor is shown when in the presence of PI(3,4)P₂. The residues interacting with the pseudoligand which are non-lipid binding residues are shown. The pseudoligand is predicted to be competed out of the lipid binding pocket when PI(3,4)P₂ is present. FIG. 9D) The predicted interaction between the mutant R211L Tapp1 PH domain and the pseudoligand of the InPTapp biosensor is shown. The R211L mutation is shown, lipid binding residues which interact with the pseudoligand are shown, and non-lipid binding residues which interact with the pseudoligand are shown.

FIGS. 10A-10B. Plasma membrane InPTapp response is dependent on PI3K and PI(3,4)P2. FIG. 10A) Average cell curves (left) of PDGF stimulated InPTapp-Kras response in serum starved NIH3T3 cells +/−PIK-75 pretreatment. Quantified response represented in a bar graph (right). Error bar shows mean±SEM; −PIK75, Rₘₐₓ=8.6±0.7%, n=18; +PIK75, Rₘₐₓ<0.01±0.8%, n=13; ****, P<0.0001, unpaired t-test with Welch's correction. Data from three independent experiments. FIG. 10B) InPTapp-Kras responses plotted against RFP fluorescence intensities in individual cells across three co-expressed lipid phosphatases: PTEN A4, INPP4B, INPP5E, and mCherry control by itself. X-axis-RFP intensity at the plasma membrane; y-axis-FRET Ratio of InPTapp-Kras emission ratio response at the plasma membrane.

FIGS. 11A-11D. Lysosomal sensor responses are dependent on PI3K activity and respective 3-PIs. FIG. 11A) Average cell curves (left) of PDGF stimulated Lyso-InPGrp (Rₘₐₓ=3.9±0.4%, n=12), Lyso-InPGrp with PIK75 pretreatment (Rₘₐₓ=1.0±0.3%, n=11), or mutant Lyso-InPGrp (Rₘₐₓ=1.5±0.4%, n=12) response in serum starved NIH3T3 cells. Quantified response represented in a bar graph (right). Error bar shows mean±SEM. ***, P<0.001, ****, P<0.0001, ordinary one-way ANOVA with Dunnett's multiple comparisons test. Data from three independent experiments. FIG. 11B) Average cell curves (left) of PDGF stimulated Lyso-InPTapp (Rₘₐₓ=1.7±0.3%, n=14), Lyso-InPTapp with PIK75 pretreatment (Rₘₐₓ=0.05±0.3%, n=12), or mutant Lyso-InPTapp response (Rₘₐₓ=0.0±0.2%, n=17) in serum starved NIH3T3 cells. +PIK-75 curve is baseline corrected. Quantified response represented in a bar graph (right). Error bar shows mean±SEM. *, P<0.05, ordinary one-way ANOVA with Dunnett's multiple comparisons test. Data from three independent experiments. FIG. 11C) SAM15 (sustainable activity metric at 15 mins) and t_{1/2} comparing InPGrp kinetics at the plasma membrane (−Kras) and lysosome (Lyso-). InPGrp, plasma membrane, SAM15=0.97±0.004, t_{1/2}=1.7±0.1 mins, n=30; lysosome, SAM15=0.24±0.1, t_{1/2}=2.1±0.09 mins, n=30; ****, P<0.0001; *, P<0.05; unpaired t-test with Welch's correction. FIG. 11D) SAM15 (sustainable activity metric at 15 mins) and t_{1/2} comparing InPTapp kinetics at the plasma membrane (−Kras) and lysosome (Lyso-). InPTapp, plasma membrane, SAM15=0.7±0.03, t_{1/2}=3.3±0.4 mins, n=18; lysosome, SAM15=−0.4±0.2, t_{1/2}=2.4±0.2 mins, n=30; ****, P<0.0001; ns, indicates not significant p-value P=0.0174, unpaired t-test with Welch's correction. Data shown in C and D is re-organized from FIGS. 3C-3D and error bars show mean±SD.

FIGS. 12A-12B. PIP₃ accumulates at the lysosome upon PDGF stimulation. FIG. 12A) Confocal images of NIH3T3 cells immunostained with an anti-PIP₃ (left) or anti-Rab7 (middle, lysosomal marker) antibody, and co-stained with DAPI (nuclear stain) (right) without (−) or with (+) PDGF stimulation for 10 minutes. Arrows (left) indicate areas of plasma membrane PIP3 accumulation, and arrows (right) indicate regions of PIP3 and Rab7 colocalization which appear white in the merged image. Scale bar=10 μm. FIG. 12B) Pearson's R value indicating colocalization is plotted for each cell without PDGF (−PDGF, 0.43±0.02) or 10 minutes after PDGF stimulation (+PDGF, 0.52±0.02). ***, P<0.001, unpaired t-test. Data from 3 independent experiments, bars represent mean value.

FIGS. 13A-13B. Dyngo-4a treatment does not inhibit PIP₃ and PI(3,4)P₂ generation at the plasma membrane. FIG. 13A) Average cell curves (left) of PDGF stimulated InPGrp-Kras response in serum starved NIH3T3 cells +/−Dyngo-4a pretreatment. Quantified response represented in a bar graph (right). Error bar shows mean±SEM. -Dyngo-4a, Rₘₐₓ=7.6±0.5%, n=25; +Dyngo-4a, Rₘₐₓ=8.9±0.1%, n=14; ns, not significant, P>0.05, unpaired t-test with Welch's correction. Data from three independent experiments. FIG. 13B) Average cell curves (left) of PDGF stimulated InPTapp-Kras response in serum starved NIH3T3 cells+/−Dyngo-4a pretreatment. Quantified response represented in a bar graph (right). Error bar shows mean±SEM. -Dyngo-4a, Rₘₐₓ=9.5±0.1%, n=18; +Dyngo-4a, Rₘₐₓ=6.8±0.1%, n=9; ns, not significant, P>0.05, unpaired t-test with Welch's correction. Data from three independent experiments.

FIGS. 14A-14B. Plasma membrane-targeted lipid phosphatases do not colocalize with lysosomes. FIG. 14A) Quantified Pearson's R-value of LysoTracker Red with Lyso-InPGrp or LysoTracker Green with PTENA4-Kras, INPP4B-Kras, INPP5E-Kras. Pearson correlation coefficient shows the lack of colocalization of plasma membrane-targeted phosphatases with lysosomes. Data from three independent experiments, Lyso-InPGrp, PCC=0.645±0.012, n=26; PTENA4-Kras, PCC=0.168±0.01, n=26; INPP4B-Kras, PCC=0.12±0.04, n=21; INPP5E-Kras, PCC=0.13±0.004, n=18. Ordinary one-way ANOVA followed by Dunnett's multiple comparison test. ****, P<0.0001. FIG. 14B) Confocal images of NIH3T3 cell co-expressing INPP5E-KRAS and InPGrp-2×FYVE are shown on the left. Quantified Mander's M1 value (overlap of INPP5E-KRAS with InPGrp-2×FYVE) and Pearson's R value, on the right, show little colocalization of the plasma membrane targeted phosphatase with endosomes. Scale bar=10 μm.

FIGS. 15A-15F. PIP₃ rapidly accumulates at endosomes en route to lysosomes. FIG. 15A) Endocytosis assay was conducted using ECGreen dye. Serum starved NIH3T3 expressing LAMP1-mCherry (bottom) imaged at a spinning disk confocal microscope before, during and after one minute-incubation with ECGreen (top) and PDGF. Line plots show colocalization of ECGreen and lysosomal mCherry fluorescence one minute after ECGreen and PDGF addition. Scale bar=10 μm unless otherwise noted in image. FIG. 15B) Epifluorescence images of InPGrp-2×FYVE colocalization with mCherry-Rab5 to validate its endosomal localization. Scale bar=20 μm. FIG. 15C) Maximum response (ΔR/R₀, left) and time to half maximum response (t_{1/2}, right) comparing InPGrp responses at the plasma membrane (−Kras), endosome (Endo-), and lysosome (Lyso-). Error bars show mean±standard deviation (SD). InPGrp, plasma membrane, ΔR/R0=8.89±0.6, n=26, t_{1/2}=1.7±0.1 min, n=30; Endo, ΔR/R₀=5.3±0.4, n=36, t1/2=2.2 0.2 min, n=36; Lyso, magenta, ΔR/R₀=2.8±0.3, n=30, t_{1/2}=2.07 0.09 mi n=30; ****, P<0.0001; *, P<0.05; one-way ANOVA with Dunnett's multiple comparisons. PM- and Lyso-data re-organized from FIGS. 3C-3D. Data from three independent experiments. FIG. 15D) Epifluorescence images of InPAkt-2×FYVE (left) colocalization with mCherry-Rab5 (middle) to validate its endosomal localization. Scale bar=20 μm. Average cell curves (left) of PDGF stimulated Endo-InPAkt (−PIK75, Rₘₐₓ=6.8±0.1%, n=14) or PIK75 pre-treated PDGF stimulated Endo-InPAkt (+PIK75, Rₘₐₓ=2.5±0.1%, n=17) response in serum starved NIH3T3 cells. Quantified response represented in a bar graph (right). Error bar shows mean±SEM. ****, P<0.0001; *, P<0.05; unpaired t-test with Welch's correction. Data from three independent experiments. FIG. 15E) Epifluorescence images of INPP5E-2×FYVE (middle) colocalization with EGFP-Rab5 (left) to validate its endosomal localization. Scale bar=20 μm. FIG. 15F) Average cell curves (left) of PDGF stimulated Lyso-InPAkt response in serum starved NIH3T3 cells with co-expression of INPP5E-2×FYVE or mCherry-2×FYVE. Error bar shows mean±SEM. mCherry, Rₘₐₓ=6.1±0.1%, n=18; INPP5E, Rₘₐₓ=2.7±0.6%, n=15; **, P=0.00143, unpaired t-test with Welch's correction. Data from three independent experiments.

FIGS. 16A-16D. Endosomally targeted tyrosine phosphatase PTP1B does not inhibit lysosomal lipid accumulation. FIG. 16A) Epifluorescence images of ER-InPAkt (left) colocalization with ER tracker red (middle) to validate its endoplasmic reticulum localization. Scale bar=20 μm. FIG. 16B) PTP1B expression inhibited accumulation of 3-phosphoinositides at the endoplasmic reticulum. Untargeted, mCherry-tagged PTP1B or mCherry was co-expressed with endoplasmic reticulum targeted InPAkt (ER-InPAkt). Epifluorescence images of ER-InPAkt (left) co-expressed with PTP1B (middle) are shown. Scale bar=20 μm. Serum-starved NIH3T3 cells were stimulated with PDGF and changes in the normalized FRET Ratio (Y/C) against time (left) and bar chart of maximum response for each cell (right) were plotted for each condition. Error bars show mean±SEM. mCherry, Rₘₐₓ=1.04±0.004, n=47, PTP1B, Rₘₐₓ=1.02±0.002, n=50, ***, P<0.001, unpaired t-test with Welch's correction. Data from three independent experiments. Scale bar=20 μm. FIG. 16C) Epifluorescence images of PTP1B-2×FYVE (middle) colocalization with EGFP-Rab5 (left) to validate its endosomal localization. Scale bar=20 μm. FIG. 16D) PDGF stimulated 3-phosphoinositide accumulation at the lysosome was not affected by endosomal PTP1B expression. mCherry tagged PTP1B of mCherry targeted to endosomes was co-expressed with lysosome-targeted InPAkt (Lyso-InPAkt). Serum-starved NIH3T3 cells were stimulated with PDGF and changes in the normalized FRET Ratio (Y/C) against time (left) and bar chart of maximum response for each cell (right) were plotted for each condition. Error bars show mean±SEM. mCherry, Rₘₐₓ=1.03±0.009, n=16, PTP1B, Rₘₐₓ=1.05±0.01, n=21, ns, P>0.05, unpaired t-test with Welch's correction. Data from at least three independent experiments. Scale bar=20 μm.

### DETAILED DESCRIPTION

Provided herein are engineered biosensor polypeptides for detecting a target 3-PI (e.g., PI(3,4)P₂ or PIP₃) in a cell. The biosensor polypeptides provided herein include a lipid binding domain and a pseudoligand that competes with a target 3-PI for binding to the lipid binding domain. Upon a target 3-PI (e.g., PI(3,4)P₂ or PIP₃) outcompeting a previously bound pseudoligand for binding to the lipid binding domain, such biosensor polypeptides undergo a detectable conformational change and thus can be used to assess localization patterns and dynamics of targeted phospholipids within a cell.

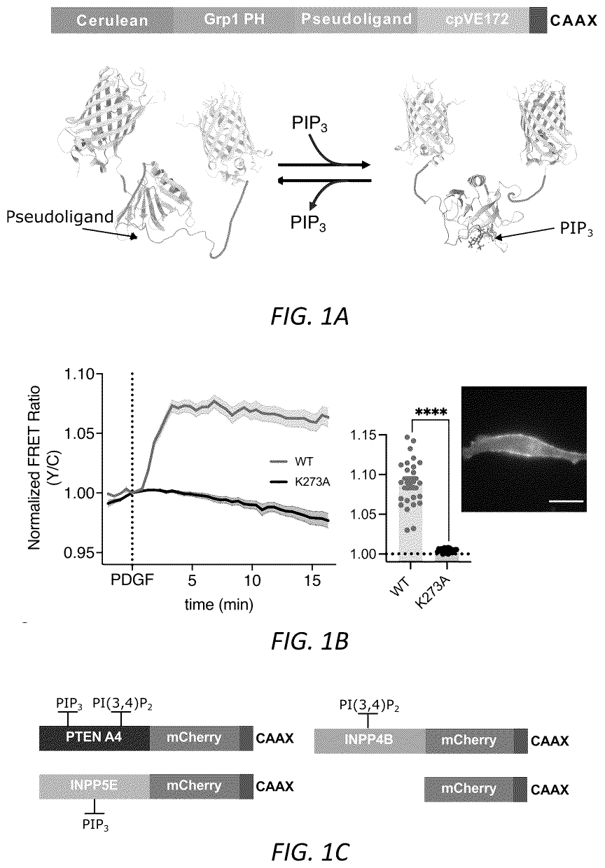

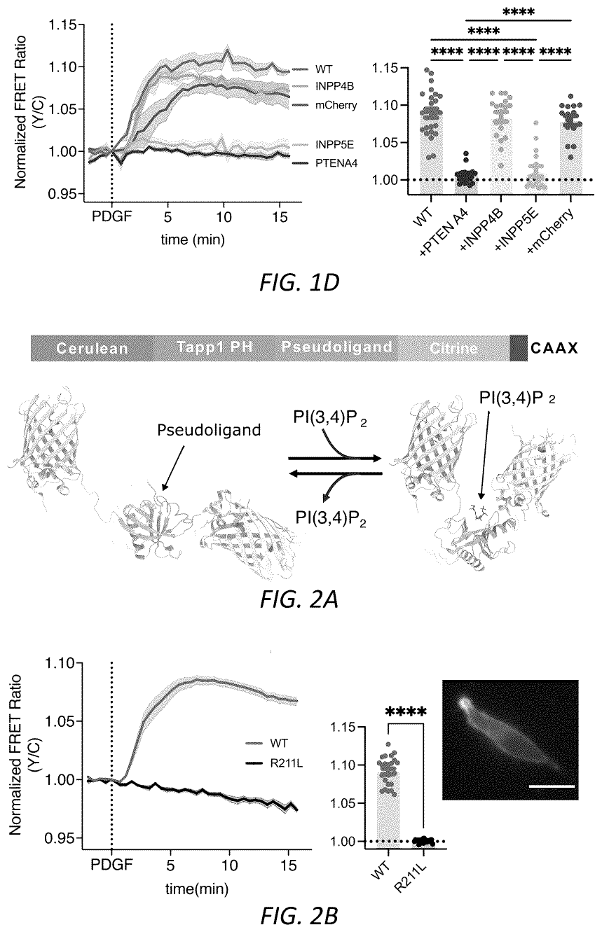

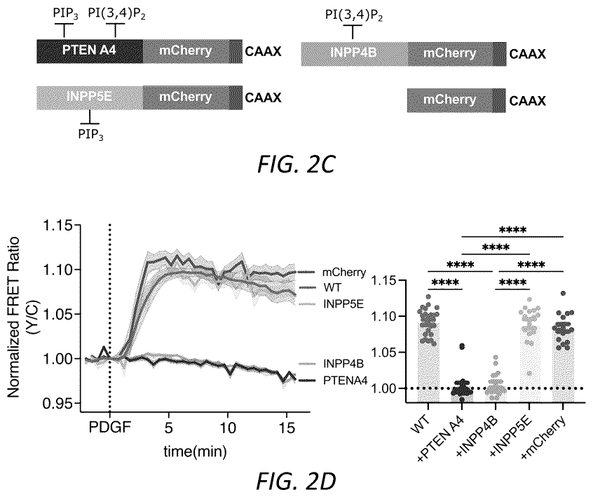

FIGS. 1A and 2A provide non-limiting exemplary schematic depictions of two embodiments of the biosensor polypeptides provided herein. As shown in FIGS. 1A and 2A, biosensor polypeptides provided herein include a lipid binding domain and a pseudoligand capable of binding to the lipid binding domain, sandwiched between a fluorophore donor and a fluorophore acceptor which undergo fluorescence resonance energy transfer (FRET). In the presence of a target 3-PI (e.g., PI(3,4)P₂ or PIP₃), the target 3-PI binds to the lipid binding domain and competes off the previously-bound pseudoligand. As shown in FIGS. 1A and 2A, binding of the target 3-PI (e.g., PI(3,4)P₂ or PIP₃) to the lipid binding domain leads to a detectable change in the conformation of the biosensor polypeptide and thereby the FRET efficiency.

Accordingly, provided herein are engineered biosensor polypeptides that can be used to assess the presence, subcellular localization, and dynamics of target 3-PIs (e.g., PI(3,4)P₂ and PIP₃). Such biosensor polypeptides allow for enhanced phospholipid targetability and the potential for affinity tuning as compared to other lipid sensors. Furthermore, these biosensor polypeptides provide opportunities for assessing cellular localization of target 3-PIs and detecting PI3K/Akt pathway signaling in a cell, for example, in response to PI3K/Akt signaling modulators (e.g., small molecule PI3K/Akt pathway inhibitors).

### I. Biosensor Polypeptides

Engineered biosensor polypeptides provided herein include a lipid binding domain, a pseudoligand, linkers, an acceptor moiety, a donor moiety, and optionally a targeting sequence.

### Lipid Binding Domains

A lipid biding domain refers to a portion of a biosensor polypeptide that is capable of being bound by a target 3-PI with high affinity.

A lipid binding domain for use in a biosensor polypeptide provided herein can include any amino acid sequence. In some embodiments, the lipid binding domain can be a pleckstrin homology (PH) lipid binding domain. For example, a lipid binding domain that can be included in a biosensor polypeptide provided herein can be a PH domain derived from a general receptor of phosphoinositides 1 (GRP1) polypeptide (e.g., a Grp1 PH domain). In another example, a lipid binding domain that can be included in a biosensor polypeptide provided herein can be a PH domain derived from a tandem PH-domain-containing protein 1 (TAPP1) polypeptide (e.g., a Tapp1 PH domain). Additional examples of polypeptides from which a PH domain that can be included in a biosensor polypeptide provided herein can be derived include, without limitation, ARNO, Btk, Cytohesin1, TAPP2, Akt1, and DAPP1. Exemplary lipid binding domains for use in a biosensor polypeptide provided herein can comprise or consist of the amino acid sequences shown in Table 1 at SEQ ID NOs:1-8. In some embodiments, a lipid binding domain for use in a biosensor polypeptide provided herein can comprise or consist of an amino acid sequence selected from SEQ ID NOs:1-8 with one or more mutations (e.g., 1, 2, 3, 4 or more mutations). In some embodiments, a lipid binding domain that can be included in a biosensor polypeptide provided herein can be as described elsewhere (e.g., Marshall et al., Mol Cell Biol., 22(15):5479-91 (2002); Frech et al., Enzymology, 272(13):8474-8481 (1997); Varnai et al., J Cell Sci, 118(20):4879-4888 (2005); and Baraldi et al., Structure, 7(4):449-460 (1999)).

TABLE 1
Exemplary lipid binding domains

| SEQ ID | | |
|---|---|---|
| NO: | Target 3-PI | Lipid Binding Domain Amino Acid Sequence |
| 1 | PIP₃ | TFFNPDREGWLLKLGGRVKTWKRRWFIL |
| | (Grp1 PH domain) | TDNCLYYFEYTTDKEPRGIIPLENLSIREV |
| | | EDPRKPNCFELYNPSHKGQVIKACKTEAD |
| | | GRVVEGNHVVYRISAPSPEEKEEWMKSIK |
| | | ASISRD |
| 2 | PI(3,4)P₂ | FTPKPPQDSAVIKAGYCVKQGAVMKNW |
| | (TAPP1 PH Domain) | KRRYFQLDENTIGYFKSELEKEPLRVIPLK |
| | | EVHKVQECKQSDIMMRDNLFEIVTTSRTF |
| | | YVQADSPEEMHSWIKAVSGAIVAQRGPG |
| | | RSASSEHP |
| 3 | PIP₃ | NEPFKIPEDDGNDLTHTFFNPDREGWLLK |
| | (ARNO PH Domain) | LGGGRVKTWKRRWFILTDNCLYYFEYTT |
| | | DKEPRGIIPLENLSIREVDDPRKPNCFELYI |
| | | PNNKGQLIKACKTEADGRVVEGNHMVY |
| | | RISAPTQEEKDEWIKSIQAAVSVDPFYEM |
| | | LAARKKRISVKKKQEQ |
| 4 | PIP₃ | MAAVILESIFLKRSQQKKKTSPLNFKKRL |
| | (Btk PH Domain) | FLLTVHKLSYYEYDFERGRRGSKKGSIDV |
| | | EKITCVETVVPEKNPPPERQIPRRGEESSE |
| | | MEQISIIERFPYPFQVVYDEGPLYVFSPTEE |
| | | LRKRWIHQLKNVIRYNSDLVQKYHPCFW |
| | | IDGQYLCCSQTAKNAMGC |
| 5 | PIP₃ | PDREGWLLKLGGGRVKTWKRRWFILTD |
| | (Cytohesin PH | NCLYYFEYTTDKEPRGIIPLENLSIREVED |
| | Domain) | SKKPNCFELYIPDNKDQVIKACKTEADGR |
| | | VVEGNHTVYRISAPTPEEKEEWIKCIKAAI |
| | | S |
| 6 | PI(3,4)P₂ | LIKSGYCVKQGNVRKSWKRRFFALDDFTI |
| | (TAPP2 PH Domain) | CYFKCEQDREPLRTIFLKDVLKTHECLVK |
| | | SGDLLMRDNLFEIITSSRTFYVQADSPED |
| | | MHSWIKEIGAAVQ |
| 7 | PI(3,4)P₂ and PIP₃ | MSDVAIVKEGWLHKRGEYIKTWRPRYFL |
| | (Akt1 PH Domain) | LKNDGTFIGYKERPQDVDQREAPLNNFSV |
| | | AQCQLMKTERPRPNTFIIRCLQWTTVIERT |
| | | FHVETPEEREEWTTAIQTVADGLKKQEEE |
| | | EMDFRSGSPSDNSGAEEMEVSLAKPKHR |
| | | VTMNEFEYLKLLGKGTFGKV |
| 8 | PI(3,4)P₂ and PIP₃ | LGTKEGYLTKQGGLVKTWKTRWFTLHR |
| | (DAPP1 PH Domain) | NELKYFKDQMSPEPIRILDLTECSAVQFD |
| | | YSQERVNCFCLVFPFRTFYLCAKTGVEAD |
| | | EWIKILRWKLS |

### Pseudoligands

A pseudoligand refers to a portion of a biosensor polypeptide that is capable of binding to the lipid binding domain of the biosensor polypeptide. In the presence of a target 3-PI (e.g., PI(3,4)P₂ or PIP₃), a pseudoligand is outcompeted by the target 3-PI for binding to the lipid biding domain of the biosensor polypeptide.

A pseudoligand for use in a biosensor polypeptide provided herein can include any amino acid sequence. In some embodiments, a pseudoligand can comprise or consist of the amino acid sequence set forth in SEQ ID NO:9 (VAEEEDDEEEDEDD (SEQ ID NO:9)). In some embodiments, a pseudoligand for use in a biosensor polypeptide provided herein can comprise or consist of the amino acid sequence set forth in SEQ ID NO:9 with one or more mutations (e.g., 1, 2, 3, 4 or more mutations).

### Linkers

A biosensor polypeptide provided herein can include one or more linkers. In some embodiments, the lipid binding domain and pseudoligand of a biosensor polypeptide provided herein can be flanked on one or both ends (e.g., the amino terminus (N-terminus) and/or the carboxy terminus (C-terminus)) by a linker. For example, a biosensor polypeptide provided herein can include, in order from N-terminus to C-terminus, a first linker, a lipid binding domain, a second linker, a pseudoligand, and a third linker.

A linker for use in a biosensor polypeptide provided herein can include any amino acid sequence. In some embodiments, the linker can be a flexible linker. For example, a linker that can be included in a biosensor provided herein can be a serine and glycine rich flexible linker. Exemplary linkers for use in a biosensor polypeptide provided herein can comprise or consist of the amino acid sequences shown in Table 2 at SEQ ID NOs:10-13. In some embodiments, a linker for use in a biosensor polypeptide provided herein can comprise or consist of an amino acid sequence selected from SEQ ID NOs:10-13 with one or more mutations (e.g., 1, 2, 3, 4 or more mutations).

TABLE 2
Exemplary linkers

| SEQ ID | |
|---|---|
| NO: | Linker Amino Acid Sequences |
| 10 | RMH |
| 11 | SAGGS |
| 12 | GGSEL |
| 13 | (GGS)ₙ |

### Donor and Acceptor Moieties

A biosensor polypeptide provided herein can include one or more donor and acceptor moieties (e.g., one or more donor and acceptor moiety pairs) (e.g., one or more donor and acceptor moieties known in the art or described herein). A donor moiety and an acceptor moiety can be included anywhere in a biosensor polypeptide provided herein. In some embodiments, a biosensor polypeptide provided herein can include a donor moiety at the N-terminus of the biosensor polypeptide and an acceptor moiety at the C-terminus of the biosensor polypeptide. For example, a biosensor polypeptide provided herein can include, in order from N-terminus to C-terminus, a donor moiety, a first linker, a lipid binding domain, a second linker, a pseudoligand, a third linker, and an acceptor moiety. In some embodiments, a biosensor polypeptide provided herein can include an acceptor moiety at the N-terminus of the biosensor polypeptide and a donor moiety at the C-terminus of the biosensor polypeptide. For example, a biosensor polypeptide provided herein can include, in order from N-terminus to C-terminus, an acceptor moiety, a first linker, a lipid binding domain, a second linker, a pseudoligand, a third linker, and a donor moiety.

In some embodiments, the donor moiety comprises a fluorophore donor and the acceptor moiety comprises a fluorophore acceptor (e.g., a fluorescence resonance energy transfer (FRET) pair). Any fluorophore donor and acceptor moiety suitable for FRET can be used in a biosensor polypeptide provided herein. Fluorophore donor and acceptor moiety pairs for use in a biosensor polypeptide provided herein are known in the art and are commercially available (e.g., Sakar et al., Journal of Biomedical Optics, 14(3):034047 (2009); Heim, Prasher, and Tsien, PNAS, 91(26):12501-12504 (1994); Nagai et al., PNAS, 101(29):10554-10559; Ross et al., eLife, 7:e35458 (2018); Griesbeck et al., JBC, 276(31):29188-29194 (2001), and Bajar et al., Sensors (Basel), 16(9):1488 (2016)).

As used herein, a “fluorophore donor” refers to a fluorophore that, upon absorbing light, can transfer excitation energy to a fluorophore acceptor. In some embodiments, a fluorophore donor for use in a biosensor polypeptide provided herein can be a genetically encodable fluorescent protein. Non-limiting examples of genetically encodable fluorescent proteins for use as a fluorophore donor in a biosensor polypeptide provided herein include cerulean, cyan (e.g., enhanced cyan fluorescent protein (ECFP)), blue, green, yellow, red, mT-Sapphire, mTagBFP, mAmertrine, mTurquoise2, CyPet, and LSSmOrange fluorescent polypeptides. In some embodiments, a fluorophore donor for use in a biosensor polypeptide provided herein can be a self-labeling protein labeled with a small molecule fluorophore (e.g., HaloTag and/or SNAPtag). Exemplary fluorophore donors for use in a biosensor polypeptide provided herein can comprise or consist of the amino acid sequences shown in Table 3 at SEQ ID NOs: 14-26. In some embodiments, a fluorophore donor for use in a biosensor polypeptide provided herein can comprise or consist of an amino acid sequence selected from SEQ ID NOs:14-26 with one or more mutations (e.g., 1, 2, 3, 4 or more mutations).

TABLE 3
Exemplary fluorophore donors

| SEQ ID | | |
|---|---|---|
| NO: | Fluorophore Donor | Fluorophore Donor Amino Acid Sequence |
| 14 | Cerulean Fluorescent | MVSKGEELFTGVVPILVELDGDVNGHRFS |
| | Protein | VSGEGEGDATYGKLTLKFICTTGKLPVPW |
| | | PTLVTTLTWGVQCFARYPDHMKQHDFFK |
| | | SAMPEGYVQERTIFFKDDGNYKTRAEVK |
| | | FEGDTLVNRIELKGIDFKEDGNILGHKLE |
| | | YNAISDNVYITADKQKNGIKAHFKIRHNI |
| | | EDGSVQLADHYQQNTPIGDGPVLLPDNH |
| | | YLSTQSALSKDPNEKRDHMVLLEFVTAA |
| | | GITLGMDELYK |
| 15 | Cyan Fluorescent | MVSKGEELFTGVVPILVELDGDVNGHKF |
| | Protein | SVSGEGEGDATYGKLTLKFICTTGKLPVP |
| | | WPTLVTTLSWGVQCFARYPDHMKQHDF |
| | | FKSAMPEGYVQERTIFFKDDGNYKTRAE |
| | | VKFEGDTLVNRIELKGIDFKEDGNILGHK |
| | | LEYNAIHGNVYITADKQKNGIKANFGLN |
| | | CNIEDGSVQLADHYQQNTPIGDGPVLLPD |
| | | NHYLSTQSKLSKDPNEKRDHMVLLEFVT |
| | | AAGITLGMDELYK |
| 16 | Enhanced Cyan | MVSKGEELFTGVVPILVELDGDVNGHKF |
| | Fluorescent Protein | SVSGEGEGDATYGKLTLKFICTTGKLPVP |
| | (ECFP) | WPTLVTTLTWGVQCFSRYPDHMKQHDFF |
| | | KSAMPEGYVQERTIFFKDDGNYKTRAEV |
| | | KFEGDTLVNRIELKGIDFKEDGNILGHKL |
| | | EYNYISHNVYITADKQKNGIKANFKIRHN |
| | | IEDGSVQLADHYQQNTPIGDGPVLLPDNH |
| | | YLSTQSALSKDPNEKRDHMVLLEFVTAA |
| | | GITLGMDELYK |
| 17 | Blue Fluorescent | MSKGEELFTGVVPILVELDGDVNGHKFS |
| | Protein | VSGEGEGDATYGKLTLKFICTTGKLPVPW |
| | | PTLVTTFSHGVQCFSRYPDHMKQHDFFKS |
| | | AMPEGYVQERTIFFKDDGNYKTRAEVKF |
| | | EGDTLVNRIELKGIDFKEDGNILGHKLEY |
| | | NFNSHNVYIMADKQKNGIKVNFKIRHNIE |
| | | DGSVQLADHYQQNTPIGDGPVLLPDNHY |
| | | LSTQSALSKDPNEKRDHMVLLEFVTAAGI |
| | | THGMDELYK |
| 18 | Green Fluorescent | MSKGEELFTGVVPILVELDGDVNGHKFS |
| | Protein | VSGEGEGDATYGKLTLKFICTTGKLPVPW |
| | | PTLVTTFSYGVQCFSRYPDHMKQHDFFKS |
| | | AMPEGYVQERTIFFKDDGNYKTRAEVKF |
| | | EGDTLVNRIELKGIDFKEDGNILGHKLEY |
| | | NYNSHNVYIMADKQKNGIKVNFKIRHNIE |
| | | DGSVQLADHYQQNTPIGDGPVLLPDNHY |
| | | LSTQSALSKDPNEKRDHMVLLGFVTAAGI |
| | | THGMDELYK |
| 19 | Yellow Fluorescent | MVSKGEELFTGVVPILVELDGDVNGHKF |
| | Protein | SVRGEGEGDATNGKLTLKLISTTGKLPVP |
| | | WPTLVTTLGYGLMVFARYPDHMKQHDF |
| | | FKSAMPEGYVQERTISFEDDGYYKTRAE |
| | | VKFEGDTLVNRIVLKGIDFKEDGNILGHK |
| | | LEYNFNPHNVYITADKQKNGIKANFKIRH |
| | | NVEDGGVQLADHYQQNTPIGDGPVLMPD |
| | | NHYLSYQSKLSKDPNEKRDHMVLKERVT |
| | | AAGITHDMNELYK |
| 20 | Red Fluorescent | MASSEDVIKEFMRFKVRMEGSVNGHEFEI |
| | Protein | EGEGEGRPYEGTQTAKLKVTKGGPLPFA |
| | | WDILSPQFQYGSKAYVKHPADIPDYLKLS |
| | | FPEGFKWERVMNFEDGGVVTVTQDSSLQ |
| | | DGEFIYKVKLRGTNFPSDGPVMQKKTMG |
| | | WEASTERMYPEDGALKGEIKMRLKLKDG |
| | | GHYDAEVKTTYMAKKPVQLPGAYKTDI |
| | | KLDITSHNEDYTIVEQYERAEGRHSTGA |
| 21 | mT-Sapphire | MVSKGEELFTGVVPILVELDGDVNGHKF |
| | Fluorescent Protein | SVSGEGEGDATYGKLTLKFICTTGKLPVP |
| | | WPTLVTTFSYGVMVFARYPDHMKQHDF |
| | | FKSAMPEGYVQERTIFFKDDGNYKTRAE |
| | | VKFEGDTLVNRIELKGIDFKEDGNILGHK |
| | | LEYNFNSHNVYIMADKQKNGIKANFKIR |
| | | HNIEDGGVQLADHYQQNTPIGDGPVLLP |
| | | DNHYLSIQSALSKDPNEKRDHMVLLEFVT |
| | | AAGITLGMDELYK |
| 22 | mTagBFP Fluorescent | MSELIKENMHMKLYMEGTVDNHHFKCT |
| | Protein | SEGEGKPYEGTQTMRIKVVEGGPLPFAFD |
| | | ILATSFLYGSKTFINHTQGIPDFFKQSFPEG |
| | | FTWERVTTYEDGGVLTATQDTSLQDGCL |
| | | IYNVKIRGVNFTSNGPVMQKKTLGWEAF |
| | | TETLYPADGGLEGRNDMALKLVGGSHLI |
| | | ANIKTTYRSKKPAKNLKMPGVYYVDYRL |
| | | ERIKEANNETYVEQHEVAVARYCDLPSK |
| | | LGHKLN |
| 23 | mAmetrine Fluorescent | MVSKGEELFTGVVPILVELDGDVNGHKF |
| | Protein | SVRGEGEGDATNGKLTLKFICTSGKLPVP |
| | | WPTLVTTLSYGVQCFARYPDHMKQHDFF |
| | | KSAMPEGYVQERTISFKDDGSYRTRAEV |
| | | KFEGDTLVNRIELKGIDFKEDGNILGHKL |
| | | EYNMNVWDAYITADKQKNGIKANFKIEH |
| | | NVEDGGVQLADAYQQNTPIGDGSVLLPD |
| | | NHYLSFQSKLFKDPNEQRDHMVLLEFVT |
| | | AAGITPGMDELYK |
| 24 | m Turquoise2 | MVSKGEELFTGVVPILVELDGDVNGHKF |
| | Fluorescent Protein | SVSGEGEGDATYGKLTLKFICTTGKLPVP |
| | | WPTLVTTLSWGVQCFARYPDHMKQHDF |
| | | FKSAMPEGYVQERTIFFKDDGNYKTRAE |
| | | VKFEGDTLVNRIELKGIDFKEDGNILGHK |
| | | LEYNYFSDNVYITADKQKNGIKANFKIRH |
| | | NIEDGGVQLADHYQQNTPIGDGPVLLPD |
| | | NHYLSTQSKLSKDPNEKRDHMVLLEFVT |
| | | AAGITLGMDELYK |
| 25 | CyPet Fluorescent | MVSKGEELFGGIVPILVELEGDVNGHKFS |
| | Protein | VSGEGEGDATYGKLTLKFICTTGKLPVPW |
| | | PTLVTTLTWGVQCFSRYPDHMKQHDFFK |
| | | SVMPEGYVQERTIFFKDDGNYKTRAEVK |
| | | FEGDTLVNRIELKGIDFKEDGNILGHKLE |
| | | YNYISHNVYITADKQKNGIKANFKARHNI |
| | | TDGSVQLADHYQQNTPIGDGPVILPDNHY |
| | | LSTQSALSKDPNEKRDHMVLLEFVTAAGI |
| | | THGMDELYK |
| 26 | LSSmOrange | MVSKGEENNMAIIKEFMRFKVRMEGSVN |
| | Fluorescent Protein | GHEFEIEGEGEGRPYEGFQTVKLKVTKGG |
| | | PLPFAWDILSPQFTYGSKAYVKHPADIPD |
| | | YLKLSFPEGFKWERVMNFEDGGVVTVTQ |
| | | DSSLQDGEFIYKVKLRGTNFPSDGPVMQK |
| | | KTMGMEASSERMYPEDGALKGEDKLRL |
| | | KLKDGGHYTSEVKTTYKAKKPVQLPGAY |
| | | IVDIKLDITSHNEDYTIVEQYERAEGRHST |
| | | GGMDELYK |

As used herein, a “fluorophore acceptor” refers to a fluorophore that can accept energy transferred by a fluorophore donor and use the transferred energy to emit light at its own characteristic emission wavelength spectrum. In some embodiments, a fluorophore acceptor for use in a biosensor polypeptide provided herein can be a genetically encodable fluorescent protein. Non-limiting examples of genetically encodable fluorescent proteins for use as a fluorophore acceptor in a biosensor polypeptide provided herein include citrine, cpVenus (VE172), tdTomato, sfGFP, mVenus, EYFP, mNeonGreen, sREACh1, YPet, mKate2, green, yellow, orange, red, and far-red fluorescent polypeptides. In some embodiments, a fluorophore acceptor for use in a biosensor polypeptide provided herein can be a self-labeling protein labeled with a small molecule fluorophore (e.g., HaloTag and/or SNAPtag). Exemplary fluorophore acceptors for use in a biosensor polypeptide provided herein can comprise or consist of the amino acid sequences shown in Table 4 at SEQ ID NOs:27-42. In some embodiments, a fluorophore acceptor for use in a biosensor polypeptide provided herein can comprise or consist of an amino acid sequence selected from SEQ ID NOs:27-42 with one or more mutations (e.g., 1, 2, 3, 4 or more mutations).

TABLE 4
Exemplary fluorophore acceptors

| SEQ ID | | |
|---|---|---|
| NO: | Fluorophore Donor | Fluorophore Donor Amino Acid Sequence |
| 27 | cp Venus[E172] | MGGVQLADHYQQNTPIGDGPVLLPDNHY |
| | | LSYQSKLSKDPNEKRDHMVLLEFVTAAGI |
| | | TLGMDELYKGGTGGSMVSKGEELFTGVV |
| | | PILVELDGDVNGHKFSVSGEGEGDATYG |
| | | KLTLKLICTTGKLPVPWPTLVTTLGYGLQ |
| | | CFARYPDHMKQHDFFKSAMPEGYVQERT |
| | | IFFKDDGNYKTRAEVKFEGDTLVNRIELK |
| | | GIDFKEDGNILGHKLEYNYNSHNVYITAD |
| | | KQKNGIKANFKIRHNIE |
| 28 | Citrine | MVSKGEELFTGVVPILVELDGDVNGHKF |
| | | SVSGEGEGDATYGKLTLKFICTTGKLPVP |
| | | WPTLVTTFGYGLMCFARYPDHMKQHDF |
| | | FKSAMPEGYVQERTIFFKDDGNYKTRAE |
| | | VKFEGDTLVNRIELKGIDFKEDGNILGHK |
| | | LEYNYNSHNVYIMADKQKNGIKVNFKIR |
| | | HNIEDGSVQLADHYQQNTPIGDGPVLLPD |
| | | NHYLSYQSALSKDPNEKRDHMVLLEFVT |
| | | AAGITLGMDELYK |
| 29 | tdTomato | MVSKGEEVIKEFMRFKVRMEGSMNGHE |
| | | FEIEGEGEGRPYEGTQTAKLKVTKGGPL |
| | | PFAWDILSPQFMYGSKAYVKHPADIPD |
| | | YKKLSFPEGFKWERVMNFEDGGLVTVT |
| | | QDSSLQDGTLIYKVKMRGTNFPPDGPV |
| | | MQKKTMGWEASTERLYPRDGVLKGEI |
| | | HQALKLKDGGHYLVEFKTIYMAKKPVQ |
| | | LPGYYYVDTKLDITSHNEDYTIVEQYER |
| | | SEGRHHLFLGHGTGSTGSGSSGTASSED |
| | | NNMAVIKEFMRFKVRMEGSMNGHEFEI |
| | | EGEGEGRPYEGTQTAKLKVTKGGPLPFA |
| | | WDILSPQFMYGSKAYVKHPADIPDYKK |
| | | LSFPEGFKWERVMNFEDGGLVTVTQDS |
| | | SLQDGTLIYKVKMRGTNFPPDGPVMQK |
| | | KTMGWEASTERLYPRDGVLKGEIHQAL |
| | | KLKDGGHYLVEFKTIYMAKKPVQLPGY |
| | | YYVDTKLDITSHNEDYTIVEQYERSEGR |
| | | HHLFLYGMDELYK |
| 30 | sfGFP | MSKGEELFTGVVPILVELDGDVNGHKFS |
| | | VRGEGEGDATNGKLTLKFICTTGKLPVP |
| | | WPTLVTTLTYGVQCFSRYPDHMKRHDF |
| | | FKSAMPEGYVQERTISFKDDGTYKTRA |
| | | EVKFEGDTLVNRIELKGIDFKEDGNILGH |
| | | KLEYNFNSHNVYITADKQKNGIKANFKI |
| | | RHNVEDGSVQLADHYQQNTPIGDGPVL |
| | | LPDNHYLSTQSVLSKDPNEKRDHMVLL |
| | | EFVTAAGITHGMDELYK |
| 31 | mVenus | MVSKGEELFTGVVPILVELDGDVNGHKF |
| | | SVSGEGEGDATYGKLTLKLICTTGKLPV |
| | | PWPTLVTTLGYGLQCFARYPDHMKQH |
| | | DFFKSAMPEGYVQERTIFFKDDGNYKTR |
| | | AEVKFEGDTLVNRIELKGIDFKEDGNIL |
| | | GHKLEYNYNSHNVYITADKQKNGIKAN |
| | | FKIRHNIEDGGVQLADHYQQNTPIGDGP |
| | | VLLPDNHYLSYQSKLSKDPNEKRDHMV |
| | | LLEFVTAAGITLGMDELYK |
| 32 | EYFP | MVSKGEELFTGVVPILVELDGDVNGHKF |
| | | SVSGEGEGDATYGKLTLKFICTTGKLPV |
| | | PWPTLVTTFGYGLQCFARYPDHMKQH |
| | | DFFKSAMPEGYVQERTIFFKDDGNYKTR |
| | | AEVKFEGDTLVNRIELKGIDFKEDGNIL |
| | | GHKLEYNYNSHNVYIMADKQKNGIKV |
| | | NFKIRHNIEDGSVQLADHYQQNTPIGDG |
| | | PVLLPDNHYLSYQSALSKDPNEKRDHM |
| | | VLLEFVTAAGITLGMDELYK |
| 33 | mNeonGreen | MVSKGEEDNMASLPATHELHIFGSINGV |
| | | DFDMVGQGTGNPNDGYEELNLKSTKG |
| | | DLQFSPWILVPHIGYGFHQYLPYPDGMS |
| | | PFQAAMVDGSGYQVHRTMQFEDGASLT |
| | | VNYRYTYEGSHIKGEAQVKGTGFPADG |
| | | PVMTNSLTAADWCRSKKTYPNDKTIIST |
| | | FKWSYTTGNGKRYRSTARTTYTFAKPM |
| | | AANYLKNQPMYVFRKTELKHSKTELNF |
| | | KEWQKAFTDVMGMDELYK |
| 34 | SREACh1 | MVSKGEELFTGVVPILVELDGDVNGHKF |
| | | SVSGEGEGDATYGKLTLKLICTTGKLPV |
| | | PWPTLVTTFGYGLMCFARYPDHMKQH |
| | | DFFKSAMPEGYVQERTIFFKDDGNYKTR |
| | | AEVKFEGDTLVNRIELKGIDFKEDGNIL |
| | | GHKLEYAWPVVNVYIMADKQKNGIKV |
| | | NFKIRHNIEDGSVQLADHYQQNTPIGDG |
| | | PVLLPDNHYLSYQSKLSKDPNEKRDHM |
| | | VLLEFVTAAGITLGMDELYK |
| 35 | YPet | MVSKGEELFTGVVPILVELDGDVNGHKF |
| | | SVSGEGEGDATYGKLTLKLLCTTGKLP |
| | | VPWPTLVTTLGYGVQCFARYPDHMKQ |
| | | HDFFKSAMPEGYVQERTIFFKDDGNYKT |
| | | RAEVKFEGDTLVNRIELKGIDFKEDGNI |
| | | LGHKLEYNYNSHNVYITADKQKNGIKA |
| | | NFKIRHNIEDGGVQLADHYQQNTPIGD |
| | | GPVLLPDNHYLSYQSALFKDPNEKRDH |
| | | MVLLEFLTAAGITEGMNELYK |
| 36 | mKate2 | MSELIKENMHMKLYMEGTVNNHHFKC |
| | | TSEGEGKPYEGTQTMRIKVVEGGPLPFA |
| | | FDILATSFMYGSKTFINHTQGIPDFFKQS |
| | | FPEGFTWERVTTYEDGGVLTATQDTSL |
| | | QDGCLIYNVKIRGVNFPSNGPVMQKKTL |
| | | GWEASTEMLYPADGGLEGRSDMALKL |
| | | VGGGHLICNLKTTYRSKKPAKNLKMPG |
| | | VYYVDRRLERIKEADKETYVEQHEVAV |
| | | ARYCDLPSKLGHK |
| 37 | GFP | MSKGEELFTGVVPILVELDGDVNGHKFS |
| | | VSGEGEGDATYGKLTLKFICTTGKLPVP |
| | | WPTLVTTFSYGVQCFSRYPDHMKQHDF |
| | | FKSAMPEGYVQERTIFFKDDGNYKTRA |
| | | EVKFEGDTLVNRIELKGIDFKEDGNILGH |
| | | KLEYNYNSHNVYIMADKQKNGIKVNF |
| | | KIRHNIEDGSVQLADHYQQNTPIGDGPV |
| | | LLPDNHYLSTQSALSKDPNEKRDHMVL |
| | | LGFVTAAGITHGMDELYK |
| 38 | YFP | MVSKGEELFTGVVPILVELDGDVNGHKF |
| | | SVRGEGEGDATNGKLTLKLISTTGKLPV |
| | | PWPTLVTTLGYGLMVFARYPDHMKQH |
| | | DFFKSAMPEGYVQERTISFEDDGYYKTR |
| | | AEVKFEGDTLVNRIVLKGIDFKEDGNIL |
| | | GHKLEYNFNPHNVYITADKQKNGIKAN |
| | | FKIRHNVEDGGVQLADHYQQNTPIGDG |
| | | PVLMPDNHYLSYQSKLSKDPNEKRDHM |
| | | VLKERVTAAGITHDMNELYK |
| 39 | Orange Fluorescent | MNLSKNVSVSVYMKGNVNNHEFEYDG |
| | Protein | EGGGDPYTGKYSMKMTLRGQNCLPFS |
| | | YDIITTAFQYGFRVFTKYPEGIVDYFKDS |
| | | LPDAFQWNRRIVFEDGGVLNMSSDITYK |
| | | DNVLHGDVWAVGVNFPPNGPVMKNEI |
| | | VMEEPTEETFTPKNGVLVGFCPKAYLL |
| | | KDGSYYYGNMTTFYRSKKSGQAPPGYH |
| | | FVKHRLVKTNVGHGFKTVEQTEYATA |
| | | HVSDLPK |
| 40 | Red Fluorescent | MASSEDVIKEFMRFKVRMEGSVNGHEF |
| | Protein | EIEGEGEGRPYEGTQTAKLKVTKGGPLP |
| | | FAWDILSPQFQYGSKAYVKHPADIPDYL |
| | | KLSFPEGFKWERVMNFEDGGVVTVTQD |
| | | SSLQDGEFIYKVKLRGTNFPSDGPVMQ |
| | | KKTMGWEASTERMYPEDGALKGEIKM |
| | | RLKLKDGGHYDAEVKTTYMAKKPVQL |
| | | PGAYKTDIKLDITSHNEDYTIVEQYERA |
| | | EGRHSTGA |
| 41 | mi-RFP | MVAGHASGSPDFGTADPSDCEREEIHLA |
| | | GSIQPHGTLLVVSEPDHRIIQASANAAEF |
| | | LNLGSVLGVPLAEIDGDLLIKILPHLDPT |
| | | AEGMPVAVRCRIGNPSTEYDGLMHRPP |
| | | EGGLIIELERAGPPIDLSGTLAPALERIRT |
| | | AGSLRALCDDTALLFQQCTGYDRVMVY |
| | | RFDEQGHGEVYSEIHVTGLESYFGNRYP |
| | | SSLVPQMARRLYERQRVRVLVDVSYQP |
| | | VPLEPRLSPLTGRDLDMSGCFLRSMSPT |
| | | HLQFLKNMGVRATLVVSLVVGGKLWG |
| | | LVICHHYLPRFIHFELRAICELLAEAIAT |
| | | RITAL |
| 42 | smuRFP | MAKTSEQRVNIATLLTENKKKIVDKASQ |
| | | DLWRRHPDLIAPGGIAFSQRDRALCLRD |
| | | YGWFLHLITFCLLAGDKGPIESIGLISIRE |
| | | MYNSLGVPVPAMMESIRCLKEASLSLL |
| | | DEEDANETAPYFDYIIKAMS |

It should be understood that a fluorophore can be a fluorophore donor when paired with one fluorophore, and it can be a fluorophore acceptor when paired with another fluorophore.

In some embodiments, the donor moiety comprises a chemiluminescence donor and the acceptor moiety comprises a chemiluminescence acceptor (e.g., a chemiluminescence resonance energy transfer (CRET) pair). In some embodiments, the donor moiety comprises a chemiluminescence donor and a non-chemiluminescence acceptor. For example, a biosensor polypeptide provided herein can include a chemiluminescence donor and a fluorophore acceptor. Any chemiluminescence donor and acceptor moiety suitable for CRET can be used in a biosensor polypeptide provided herein. In some embodiments, methods involve use of CRET and one or more of DNAzyme, RNAzyme, horseradish peroxidase (HRP)-conjugated gold nanoparticles, and HRP-conjugated antibodies.

In some embodiments, the donor moiety comprises a bioluminescence donor and the acceptor moiety comprises a bioluminescence acceptor (e.g., a bioluminescence resonance energy transfer (BRET) pair). In some embodiments, the donor moiety comprises a bioluminescence donor and a non-bioluminescence acceptor. For example, a biosensor polypeptide provided herein can include a bioluminescence donor and a fluorophore acceptor. Any bioluminescence donor and acceptor moiety suitable for BRET can be used in a biosensor polypeptide provided herein.

Chemiluminescence and bioluminescence donor and acceptor moiety pairs for use in biosensor polypeptides provided herein are known in the art (see, e.g., Wu and Jian, Micromachines (Basel), 13(10):1789 (2022); and De, Arora, and Jasani, Engineering in Translational Medicine, Cai, W. (eds) Engineering in Translational Medicine. Springer, London). In some embodiments, methods involve use of CRET and/or BRET and one or more of Renilla luciferase, firefly luciferase, and NanoLuc.

### Targeting Sequences

In some embodiments, a biosensor polypeptide provided herein can include one or more targeting sequences. For example and without limitation, a biosensor polypeptide provided herein can include one or more targeting sequences to target the biosensor polypeptide to one or more subcellular locations. A targeting sequence can be included anywhere in a biosensor polypeptide provided herein. In some embodiments, a biosensor polypeptide provided herein can include a targeting sequence at the C-terminus of the biosensor polypeptide. For example, a biosensor polypeptide provided herein can include, in order from N-terminus to C-terminus, a donor moiety, a first linker, a lipid binding domain, a second linker, a pseudoligand, a third linker, an acceptor moiety, and a targeting sequence. In another example, a biosensor polypeptide provided herein can include, in order from N-terminus to C-terminus, a donor moiety, a first linker, a lipid binding domain, a second linker, a pseudoligand, a third linker, an acceptor moiety, a fourth linker, and a targeting sequence. In some embodiments, a biosensor polypeptide provided herein can include a targeting sequence at the N-terminus of the biosensor polypeptide. For example, a biosensor polypeptide provided herein can include, in order from N-terminus to C-terminus, a targeting sequence, a donor moiety, a first linker, a lipid binding domain, a second linker, a pseudoligand, a third linker, and an acceptor moiety. In another example, a biosensor polypeptide provided herein can include, in order from N-terminus to C-terminus, a targeting sequence, a first linker, a donor moiety, a second linker, a lipid binding domain, a third linker, a pseudoligand, a fourth linker, and an acceptor moiety.

A targeting sequence for use in a biosensor polypeptide provided herein can target the biosensor polypeptide to any subcellular location. Examples of subcellular locations that can be targeted by a biosensor polypeptide including a targeting sequence provided herein include, without limitation, the nucleus, the cytosol, the Golgi apparatus, the lysosome, the plasma membrane, the endosome, the endoplasmic reticulum, and the mitochondria. Exemplary targeting sequences for use in a biosensor polypeptide provided herein can comprise or consist of the amino acid sequences shown in Table 5 at SEQ ID NOs:43-54. In some embodiments, a targeting sequence for use in a biosensor polypeptide provided herein can comprise or consist of an amino acid sequence selected from SEQ ID NOs:43-54 with one or more mutations (e.g., 1, 2, 3, 4 or more mutations).

TABLE 5
Exemplary targeting sequences

| SEQ ID | | |
|---|---|---|
| NO: | Subcellular Target | Targeting Sequence Amino Acid Sequence |
| 43 | Lysosome | MAAPGSARRPLLLLLLLLLLGLMHCASA |
| | | AMFMVKNGNGTACIMANFSAAFSVNYD |
| | | TKSGPKNMTFDLPSDATVVLNRSSCGKE |
| | | NTSDPSLVIAFGRGHTLTLNFTRNATRYS |
| | | VQLMSFVYNLSDTHLFPNASSKEIKTVES |
| | | ITDIRADIDKKYRCVSGTQVHMNNVTVT |
| | | LHDATIQAYLSNSSFSRGETRCEQDRPSP |
| | | TTAPPAPPSPSPSPVPKSPSVDKYNVSGT |
| | | NGTCLLASMGLQLNLTYERKDNTTVTR |
| | | LLNINPNKTSASGSCGAHLVTLELHSEGT |
| | | TVLLFQFGMNASSSRFFLQGIQLNTILPD |
| | | ARDPAFKAANGSLRALQATVGNSYKCN |
| | | AEEHVRVTKAFSVNIFKVWVQAFKVEG |
| | | GQFGSVEECLLDENSMLIPIAVGGALAG |
| | | LVLIVLIAYLVGRKRSHAGYQTI |
| 44 | Plasma Membrane | CAAX |
| 45 | Plasma Membrane | KKKKKSKTKCVIM |
| 46 | Endosome | RSTSRSTARPNGQPQASKICQFKLVLLGE |
| | (Rab5) | SAVGKSSLVLRFVKGQFHEYQESTIGAA |
| | | FLTQSVCLDDTTVKFEIWDTAGQERYHS |
| | | LAPMYYRGAQAAIVVYDITNQETFARA |
| | | KTWVKELQRQASPSIVIALAGNKADLAN |
| | | KRMVEYEEAQAYADDNSLLFMETSAKT |
| | | AMNVNDLFLAIAKKLPKSEPQNLGGAA |
| | | GRSRGVDLHEQSQQNKSQCCSN |
| 47 | Endosome | MFAAERAPDWVDAEECHRCRVQFGVVT |
| | (2xFYVE) | RKHHCRACGQIFCGKCSSKYSTIPKFGIE |
| | | KEVRVCEPCYEQLNKKAQGQGSESDAM |
| | | FAAERAPDWVDAEECHRCRVQFGVVTR |
| | | KHHCRACGQIFCGKCSSKYSTIPKFGIEK |
| | | EVR VCEPCYEQLNKKA |
| 48 | Endoplasmic Reticulum | MDPVVVLGLCLSCLLLLSLWKQSYGGG |
| 49 | Mitochondria | MAIQLRSLFPLALPGMLALLGWWWFFS |
| | | RKK |
| 50 | Nucleus | MSGRGKQGGKARAKAKSRSSRAGLQFP |
| | (H2A Histone tag) | VGR VHRLLRKGNYAERVGAGAPVYMA |
| | | AVLEYLTAEILELAGNAARDNKKTRIIPR |
| | | HLQLAIRNDEELNKLLGKVTIAQGGVLP |
| | | NIQAVLLPKKTESHKAKSK |
| 51 | Nucleus | PKKKRKV |
| | (Nuclear Localization | |
| | Signal) | |
| 52 | Cytosol | LPPLERLTL |
| | (Nuclear Export Signal) | |
| 53 | Golgi Apparatus | FSEAQQQLCNTRQEVNELRKLLEEERDQ |
| | (Giantin Golgi | RVAAENALSVAEEQIRRLEHSEWDSSRT |
| | Localization Sequence) | PIIGSCGTQEQALLIDLTSNSCRRTRSGVG |
| | | WKR VLRSLCHSR TR VPLLAAIYFLMIHV |
| | | LLILCFTGH |
| 54 | Mitochondria | MAIQLRSLFPLALPGMLALLGWWWFFS |
| | (Mitochondrial Outer | RKKADP |
| | Membrane) | |

### Exemplary Biosensor Polypeptides

In some embodiments, a biosensor polypeptide provided herein can include, in order from N-terminus to C-terminus, a fluorophore donor moiety such as a cerulean fluorescent protein (e.g., a cerulean fluorescent protein that comprises or consists of the amino acid sequence set forth in SEQ ID NO: 14), a first linker (e.g., a linker that comprises or consists of the amino acid sequence set forth in SEQ ID NO: 10), a lipid binding domain such as a Grp PH lipid binding domain (e.g., a Grp PH lipid binding domain that comprises or consists of the amino acid sequence set forth in SEQ ID NO:1), a second linker (e.g., a linker that comprises or consists of the amino acid sequence set forth in SEQ ID NO: 11), a pseudoligand (e.g., a pseudoligand that comprises or consists of the amino acid sequence set forth in SEQ ID NO:9), a third linker (e.g., a linker that comprises or consists of the amino acid sequence set forth in SEQ ID NO:12), and a fluorophore acceptor moiety such as a cpVenus[E172]fluorescent protein (e.g., a cpVenus[E172]fluorescent protein that comprises or consists of the amino acid sequence set forth in SEQ ID NO:27). For example, a biosensor polypeptide provided herein can include an amino acid sequence comprising SEQ ID NO:14, followed by an amino acid sequence comprising SEQ ID NO:10, followed by an amino acid sequence comprising SEQ ID NO: 1, followed by an amino acid sequence comprising SEQ ID NO: 11, followed by an amino acid sequence comprising SEQ ID NO:9, followed by an amino acid sequence comprising SEQ ID NO:12, followed by an amino acid sequence comprising SEQ ID NO:27. In another example, a biosensor polypeptide provided herein can comprise or consist of the amino acid sequence set forth in SEQ ID NO:55.

(SEQ ID NO: 55)
MVSKGEELFTGVVPILVELDGDVNGHRFSVSGEGEGDATYGKLTLKFIC
TTGKLPVPWPTLVTTLTWGVQCFARYPDHMKQHDFFKSAMPEGYVQERT
IFFKDDGNYKTRAEVKFEGDTLVNRIELKGIDFKEDGNILGHKLEYNAI
SDNVYITADKQKNGIKAHFKIRHNIEDGSVQLADHYQQNTPIGDGPVLL
PDNHYLSTQSALSKDPNEKRDHMVLLEFVTAAGITLGMDELYKRMHTFF
NPDREGWLLKLGGRVKTWKRRWFILTDNCLYYFEYTTDKEPRGIIPLEN
LSIREVEDPRKPNCFELYNPSHKGQVIKACKTEADGRVVEGNHVVYRIS
APSPEEKEEWMKSIKASISRDSAGGSVAEEEDDEEEDEDDGGSELMGGV
QLADHYQQNTPIGDGPVLLPDNHYLSYQSKLSKDPNEKRDHMVLLEFVT
AAGITLGMDELYKGGTGGSMVSKGEELFTGVVPILVELDGDVNGHKFSV
SGEGEGDATYGKLTLKLICTTGKLPVPWPTLVTTLGYGLQCFARYPDHM
KQHDFFKSAMPEGYVQERTIFFKDDGNYKTRAEVKFEGDTLVNRIELKG
IDFKEDGNILGHKLEYNYNSHNVYITADKQKNGIKANFKIRHNIE

In some embodiments, a biosensor polypeptide provided herein can include, in order from N-terminus to C-terminus, a fluorophore donor moiety such as a cyan fluorescent protein (e.g., a cyan fluorescent protein that comprises or consists of the amino acid sequence set forth in SEQ ID NO: 15), a first linker (e.g., a linker that comprises or consists of the amino acid sequence set forth in SEQ ID NO: 10), a lipid binding domain such as a Tapp1 PH lipid binding domain (e.g., a Tapp1 PH lipid binding domain that comprises or consists of the amino acid sequence set forth in SEQ ID NO:2), a second linker (e.g., a linker that comprises or consists of the amino acid sequence set forth in SEQ ID NO: 11), a pseudoligand (e.g., a pseudoligand that comprises or consists of the amino acid sequence set forth in SEQ ID NO:9), a third linker (e.g., a linker that comprises or consists of the amino acid sequence set forth in SEQ ID NO:12), and a fluorophore acceptor moiety such as a citrine fluorescent protein (e.g., a citrine fluorescent protein that comprises or consists of the amino acid sequence set forth in SEQ ID NO:28). For example, a biosensor polypeptide provided herein can include an amino acid sequence comprising SEQ ID NO: 15, followed by an amino acid sequence comprising SEQ ID NO: 10, followed by an amino acid sequence comprising SEQ ID NO:2, followed by an amino acid sequence comprising SEQ ID NO:11, followed by an amino acid sequence comprising SEQ ID NO:9, followed by an amino acid sequence comprising SEQ ID NO: 12, followed by an amino acid sequence comprising SEQ ID NO:28. In another example, a biosensor polypeptide provided herein can comprise or consist of the amino acid sequence set forth in SEQ ID NO:56. In another example, a biosensor polypeptide provided herein can comprise or consist of the amino acid sequence set forth in SEQ ID NO:59.

(SEQ ID NO: 56)
MVSKGEELFTGVVPILVELDGDVNGHKFSVSGEGEGDATYGKLTLKFIC
TTGKLPVPWPTLVTTLSWGVQCFARYPDHMKQHDFFKSAMPEGYVQERT
IFFKDDGNYKTRAEVKFEGDTLVNRIELKGIDFKEDGNILGHKLEYNAI
HGNVYITADKQKNGIKANFGLNCNIEDGSVQLADHYQQNTPIGDGPVLL
PDNHYLSTQSKLSKDPNEKRDHMVLLEFVTAAGITLGMDELYKRMHFTP
KPPQDSAVIKAGYCVKQGAVMKNWKRRYFQLDENTIGYFKSELEKEPLR
VIPLKEVHKVQECKQSDIMMRDNLFEIVTTSRTFYVQADSPEEMHSWIK
AVSGAIVAQRGPGRSASSEHPSAGGSVAEEEDDEEEDEDDGGSELMGGV
QLADHYQQNTPIGDGPVLLPDNHYLSYQSKLSKDPNEKRDHMVLLEFVT
AAGITLGMDELYKGGTGGSMVSKGEELFTGVVPILVELDGDVNGHKFSV
SGEGEGDATYGKLTLKLICTTGKLPVPWPTLVTTLGYGLQCFARYPDHM
KQHDFFKSAMPEGYVQERTIFFKDDGNYKTRAEVKFEGDTLVNRIELKG
IDFKEDGNILGHKLEYNYNSHNVYITADKQKNGIKANFKIRHNIE
(SEQ ID NO: 59)
MVSKGEELFTGVVPILVELDGDVNGHKFSVSGEGEGDATYGKLTLKFIC
TTGKLPVPWPTLVTTLSWGVQCFARYPDHMKQHDFFKSAMPEGYVQERT
IFFKDDGNYKTRAEVKFEGDTLVNRIELKGIDFKEDGNILGHKLEYNAI
HGNVYITADKQKNGIKANFGLNCNIEDGSVQLADHYQQNTPIGDGPVLL
PDNHYLSTQSKLSKDPNEKRDHMVLLEFVTAAGITLGMDELYKRMHFTP
KPPQDSAVIKAGYCVKQGAVMKNWKRRYFQLDENTIGYFKSELEKEPLR
VIPLKEVHKVQECKQSDIMMRDNLFEIVTTSRTFYVQADSPEEMHSWIK
AVSGAIVAQRGPGRSASSEHPSAGGSVAEEEDDEEEDEDDGGSELMVSK
GEELFTGVVPILVELDGDVNGHKFSVSGEGEGDATYGKLTLKFICTTGK
LPVPWPTLVTTFGYGLMCFARYPDHMKQHDFFKSAMPEGYVQERTIFFK
DDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNH
YLSYQSALSKDPNEKRDHMVLLEFVTAAGITLGMDELYK

### II. Delivery to Cells

Engineered biosensor polypeptides provided herein can be delivered to cells using any method suitable for delivery of such polypeptides into the cells. For example, a nucleic acid sequence encoding a biosensor polypeptide provided herein can be delivered to cells using a vector (e.g., an expression plasmid or a viral vector). Provided herein are targeted expression vectors for in vitro transfection and expression of a polynucleotide that encodes a biosensor polypeptide provided herein in cells. In some embodiments, approaches include insertion of the polynucleotide into a viral vector. Non-limiting examples of viruses upon which a viral vector can be based include, recombinant retroviruses, adenovirus, adeno-associated virus, lentivirus, and herpes simplex virus-1, alphavirus, vaccinia virus, baculovirus, or recombinant bacterial or eukaryotic plasmids. Viral vectors can transfect cells directly. In some embodiments, approaches include insertion of the polynucleotide into an expression plasmid. Plasmid DNA can be delivered naked or with the help of, for example and without limitation, cationic liposomes (lipofectamine) or derivatized (e.g., antibody conjugated), cationic dendrimers, inorganic vectors (e.g., iron oxide magnetofection), lipidoids, cell-penetrating peptides, cyclodextrin polymer (CDP), polylysine conjugates, gramacidin S, artificial viral envelopes or other such intracellular carriers, as well as direct injection of the construct or CaPO₄ precipitation carried out in vitro.

### III. Assays for Detecting a Target PI-3

Aspects of the present disclosure provide methods for detecting a target PI-3 (e.g., PI(3,4)P₂ or PIP₃) in a cell. To perform the assay method provided herein, a cell is contacted with a biosensor polypeptide including a donor moiety, a first linker, a lipid binding domain, a second linker, a pseudoligand, a third linker, and an acceptor moiety provided herein (or a nucleic acid encoding a biosensor polypeptide provided herein) and a signal from the donor and acceptor moiety pair is detected. In some embodiments, the signal from the donor and acceptor moiety pair that can be detected is a change in resonance energy transfer. A change in resonance energy transfer can be quantified by the acceptor moiety emission to donor moiety emission ratio. For example, in order to detect a target PI-3 (e.g., PI(3,4)P₂ or PIP₃) in a cell, a cell is contacted with a biosensor polypeptide including a donor moiety, a first linker, a lipid binding domain, a second linker, a pseudoligand, a third linker, and an acceptor moiety provided herein (or a nucleic acid encoding a biosensor polypeptide provided herein) and the acceptor moiety emission to donor moiety emission ratio is detected, wherein an increase in the acceptor moiety emission to donor moiety emission ratio as compared to a reference ratio indicates presence of the target PI-3. In another example, in order to detect a target PI-3 (e.g., PI(3,4)P₂ or PIP₃) in a cell, a cell is contacted with a biosensor polypeptide including a donor moiety, a first linker, a lipid binding domain, a second linker, a pseudoligand, a third linker, and an acceptor moiety provided herein (or a nucleic acid encoding a biosensor polypeptide provided herein) and the acceptor moiety emission to donor moiety emission ratio is detected, wherein no change in the acceptor moiety emission to donor moiety emission ratio as compared to a reference ratio indicates absence of the target PI-3. In another example, in order to detect a target PI-3 (e.g., PI(3,4)P₂ or PIP₃) in a cell, a cell is contacted with a biosensor polypeptide including a donor moiety, a first linker, a lipid binding domain, a second linker, a pseudoligand, a third linker, and an acceptor moiety provided herein (or a nucleic acid encoding a biosensor polypeptide provided herein) and the acceptor moiety emission to donor moiety emission ratio is detected, wherein a decrease in the acceptor moiety emission to donor moiety emission ratio as compared to a reference ratio indicates the absence, degradation, and/or loss of the target PI-3. In some embodiments, the acceptor moiety emission to donor moiety emission ratio can be assessed when the donor moiety is excited (e.g., when the donor moiety is a fluorophore donor and the acceptor moiety is a fluorophore acceptor such as when the donor and acceptor moiety pair is a FRET pair). For example, when the donor and acceptor moiety pair is a FRET pair, the acceptor moiety emission to donor moiety emission ratio can be assed by: (1) obtaining the fluorescence intensity from two channels when the donor moiety is excited: (i) a FRET channel that records the emission of the acceptor moiety, and (ii) a donor direct channel that records the emission of the donor moiety, and (2) calculating the ratio between the FRET channel intensity and the donor direct channel intensity. In some embodiments, the acceptor moiety emission to donor moiety emission ratio can be assessed when the donor moiety is allowed to be in contact with a substrate (e.g., when the donor moiety is a chemiluminescence donor or a bioluminescence donor such as when the when the donor and acceptor moiety pair comprises a CRET pair or a BRET pair). In some embodiments, the reference ratio is the acceptor moiety emission to donor moiety emission ratio detected from the biosensor polypeptide prior to stimulation (e.g., nutrient or mitogenic stimulation of PI3K/Akt pathway signaling) of the cell. In some embodiments, the reference ratio is the acceptor moiety emission to donor moiety emission ratio detected from a negative control biosensor polypeptide (e.g., a biosensor polypeptide that does not bind a target PI-3).

In some embodiments, the methods provided herein can include assessing the cellular localization of a target PI-3 (e.g., PI(3,4)P₂ or PIP₃) in a cell. For example, in order to assess the cellular localization of a target PI-3 (e.g., PI(3,4)P₂ or PIP₃) in a cell, a cell is contacted with a biosensor polypeptide including a donor moiety, a first linker, a lipid binding domain, a second linker, a pseudoligand, a third linker, an acceptor moiety provided herein (or a nucleic acid encoding a biosensor polypeptide provided herein), and a targeting sequence that targets the biosensor polypeptide to one or more subcellular locations within the cell, and a signal from the donor and acceptor moiety pair (e.g., the acceptor moiety emission to donor moiety emission ratio) is assessed. If the target PI-3 (e.g., PI(3,4)P₂ and PIP₃) is present in the subcellular location targeted by the targeting sequence, a change (e.g., an increase) in the signal from the donor and acceptor moiety pair (e.g., the acceptor moiety emission to donor moiety emission ratio) will be detected. If the target PI-3 (e.g., PI(3,4)P₂ and PIP₃) is absent in the subcellular location targeted by the targeting sequence, a change in the signal from the donor and acceptor moiety pair (e.g., the acceptor moiety emission to donor moiety emission ratio) will not be detected. In some embodiments, if the target PI-3 (e.g., PI(3,4)P₂ and PIP₃) is absent, degraded, and/or lost in the subcellular location targeted by the targeting sequence, a decrease in the signal from the donor and acceptor moiety pair (e.g., the acceptor moiety emission to donor moiety emission ratio) will be detected.

PI-3 lipids (e.g., PI(3,4)P₂ and PIP₃) are important lipid second messengers of the PI3K/Akt signaling pathway, and active PI3K/Akt pathway signaling in a cell will cause a change (e.g., an increase) in the level of PI-3 lipids (e.g., PI(3,4)P₂ and PIP₃) in the cell. Thus, in some embodiments, the methods provided herein can include assessing PI3K/Akt pathway signaling in a cell. For example, in order to assess PI3K/Akt pathway signaling in a cell, a cell is contacted with a biosensor polypeptide including a donor moiety, a first linker, a lipid binding domain, a second linker, a pseudoligand, a third linker, and an acceptor moiety provided herein (or a nucleic acid encoding a biosensor polypeptide provided herein), and a signal from the donor and acceptor moiety pair (e.g., the acceptor moiety emission to donor moiety emission ratio) is assessed. If there is active PI3K/Akt pathway signaling in the cell, the level of a target PI-3 (e.g., PI(3,4)P₂ or PIP₃) in the cell will increase, and a change (e.g., an increase) in the signal from the donor and acceptor moiety pair (e.g., the acceptor moiety emission to donor moiety emission ratio) will be detected. If there no active PI3K/Akt pathway signaling in the cell, the level of a target PI-3 (e.g., PI(3,4)P₂ or PIP₃) will not increase, and no change in the signal from the donor and acceptor moiety pair (e.g., the acceptor moiety emission to donor moiety emission ratio) will be detected. In some embodiments, if there is no active PI3K/Akt pathway signaling in the cell, the level of a target PI-3 (e.g., PI(3,4)P₂ or PIP₃) will decrease as a result of degradation or loss, and a decrease in the signal from the donor and acceptor moiety pair (e.g., the acceptor moiety emission to donor moiety emission ratio) will be detected.

Stimulation (e.g., nutrient or mitogenic stimulation) of PI3K/Akt pathway signaling in a cell will cause a change (e.g., an increase) in the level of PI-3 lipids (e.g., PI(3,4)P₂ and PIP₃) in the cell. Inhibition of PI3K/Akt pathway signaling in a cell with a PI3K/Akt signaling inhibitor (e.g., small molecule PI3K/Akt signaling inhibitor) will prevent a change (e.g., an increase) in the level of PI-3 lipids (e.g., PI(3,4)P₂ and PIP₃) in the cell, even in the presence of PI3K/Akt pathway stimulation (e.g., nutrient or mitogenic stimulation). Thus, in some embodiments, the methods provided herein can be used to assess the effect of PI3K/Akt signaling modulators (e.g., small molecule PI3K/Akt signaling inhibitors) on PI3K/Akt pathway signaling in a cell. For example, in order to assess the effect of a PI3K/Akt signaling inhibitor on PI3K/Akt pathway signaling in a cell, the cell is contacted with the PI3K/Akt signaling inhibitor and a biosensor polypeptide provided herein including a donor moiety, a first linker, a lipid binding domain, a second linker, a pseudoligand, a third linker, and an acceptor moiety provided herein (or a nucleic acid encoding a biosensor polypeptide provided herein), the cell is then subjected to nutrient or mitogenic stimulation, and a signal from the donor and acceptor moiety pair (e.g., the acceptor moiety emission to donor moiety emission ratio) is assessed. If the PI3K/Akt signaling inhibitor is effective, the level of a target PI-3 (e.g., PI(3,4)P₂ or PIP₃) will not increase, and no change in the signal from the donor and acceptor moiety pair (e.g., the acceptor moiety emission to donor moiety emission ratio) will be detected. If the PI3K/Akt signaling inhibitor is not effective, the level of a target PI-3 (e.g., PI(3,4)P₂ or PIP₃) will increase, and a change (e.g., an increase) in the signal from the donor and acceptor moiety pair (e.g., the acceptor moiety emission to donor moiety emission ratio) will be detected. In some embodiments, if the PI3K/Akt signaling inhibitor is effective, the level of a target PI-3 (e.g., PI(3,4)P₂ or PIP₃) will decrease as a result of degradation or loss, and a decrease in the signal from the donor and acceptor moiety pair (e.g., the acceptor moiety emission to donor moiety emission ratio) will be detected.

Methods provided herein encompass detecting a signal from a detectable label (e.g., a donor and acceptor moiety pair) using any method known in the art or provided herein. In some embodiments, methods comprise detecting a fluorescent signal, a chemiluminescent signal, or a combination thereof. For example, a biosensor polypeptide provided herein can include a fluorophore donor and a fluorophore acceptor (e.g. a FRET pair), and detection can be performed using optical detection.

### IV. Kits

The present disclosure also provides kits for detecting a target 3-PI (e.g., PI(3,4)P₂ or PIP₃). Such kits can include a biosensor polypeptide provided herein. The biosensor polypeptide can include a lipid binding domain and a pseudoligand. In some embodiments, the biosensor polypeptide can also include, without limitation, one or more linkers, a donor moiety and an acceptor moiety, and/or one or more targeting sequences. The kit can also include instructions for practicing any of the methods provided herein. Instructions supplied in the kits of the present disclosure are typically written instructions on a label or a package insert.

The kits provided herein are in suitable packaging. Suitable packaging includes, but is not limited to, containers, bottles, vials, and flexible packaging. Kits can include additional components such as buffers and interpretive information.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The invention is further described in the following examples, which do not limit the scope of the invention described in the claims.

### Materials and Methods

### Reagents and Constructs

PDGF (P3201) was purchased from Sigma, PIK-75 (S1205), Dyngo-4a (S7163) were purchased from Selleckchem, and LysoTracker Green DND-26 (L7526) and Red DND-99 (L7528) were purchased from ThermoFisher Scientific. ECGreen (E296-10) was purchased from Dojindo. InPGrp was generated through restriction enzyme cloning. Cerulean, GRP1-PH gene, and the pseudoligand peptide sequence VAEEEDDEEEDEDD (SEQ ID NO:9) following cp Venus [E172] were subcloned into full length InPGrp using SphI, PstI, and SacI restriction enzyme sites. InPTapp was generated like InPGrp with the Tapp1 PH domain instead of Grp and Citrine instead of cpVenus [E172]. InPGrp-Kras (K273A) and InPTapp-Kras (R211L) were prepared using the Q5 quick change method (E0554). Kras and Lyso variants of InPGrp and InPTapp were prepared by subcloning a BamHIEcoRI digested fragment containing full length InPGrp and InPTapp to pcDNA3′ backbones containing a C-terminal CAAX targeting motif (KKKKKSKTKCVIM; (SEQ ID NO:45)) or an N-terminal lysosome targeting motif derived from LAMP1. The endosomal InPGrp, InPGrp mutant, and InPAkt sensors were similarly generated by subcloning a BamHI/EcoRI digested pcDNA3′ backbone of EKAR4-2×FYVE (Addgene #205513) containing the C-terminal tandem FYVE domain for endosomal targeting. mCherry-tagged phosphatases were targeted to the plasma membrane by C-terminal addition of the CAAX targeting motif by Gibson assembly using the InPTapp-Kras construct as the template for the CAAX motif and pcDNA3-Lyso-PTEN(A4)-mCherry (Addgene #184054) as the template for PTENA4-mCherry. Endosome-targeted mCherry and INPP5E were generated by C-terminal addition of the tandem FYVE domain by Gibson assembly using the InPGrp-2×FYVE (Endo-InPGrp) construct as the template. mCherry and EGFP lacking a stop codon were subcloned into Rab5 containing pcDNA3′ backbones to generate mCherry-Rab5 and EGFP-Rab5 to serve as endosomal localization controls. InPAkt was subcloned into cyp450-tag containing pcDNA3′ backbone to generate ER-targeted InPakt. pPTP1BD181A-mCherry construct (Addgene plasmid #40270) was mutated back to WT, then used to generate endosomal or lysosomal PTP1B by Gibson assembly using the InPGrp-2×FYVE or LAMP1-InPGrp construct as the template. Constructs were verified by forward and reverse sequencing.

### Cell Culture and Transfection

NIH3T3 cells (CRL-1658, ATCC) were cultured in DMEM (11885, Gibco) with 10% calf serum (30-2030, ATCC) and 1% penicillin-streptomycin (Sigma-Aldrich). Cells were routinely tested negative for mycoplasma contamination and cultured in 37° C. with 5% CO₂. For live cell imaging experiments, cells were plated onto sterile 35-mm glass-bottomed dishes (D35-14-1.5N, CellVis) and grown to to about 50% confluence overnight at 37° C. with 5% CO₂. Cells were transfected with Polyjet In Vitro DNA Transfection Reagent (SL100688) from SigmaGen Laboratories with 800 ng of DNA per dish for each imaging experiment and incubated for 18-24 hours in serum-free DMEM prior to imaging.

### Live-Cell Epifluorescence Imaging

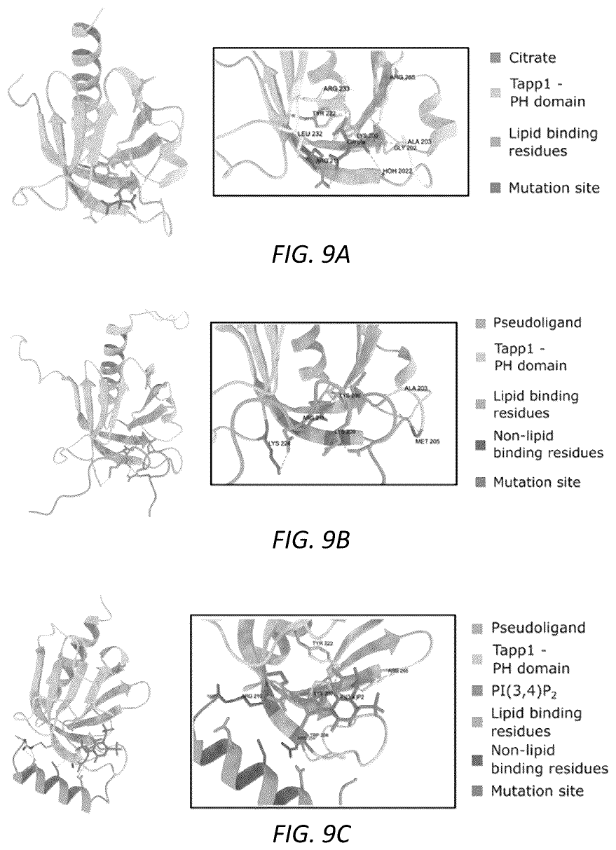

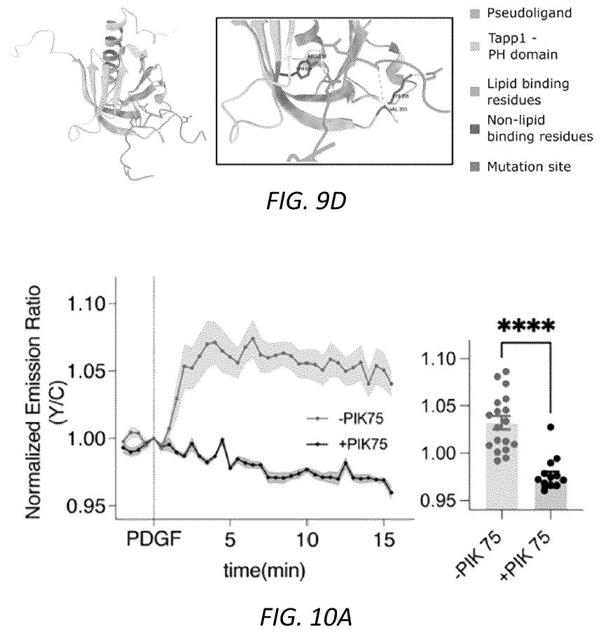

NIH3T3 cells were washed and then incubated in modified Hank's balanced salt solution, including 1×HBSS with 2 g/L glucose, pH 7.4, (10×HBSS, 14065, GIBCO), at room temperature for 10 minutes before imaging. Cells were imaged in the dark at room temperature. A Zeiss Axio Observer Z7 (Carl Zeiss) microscope with a 40×/1.4NA oil objective, Definite Focus 2 system (Carl Zeiss), and photometrics Prime95B sCMOS camera (Photometrics) were used to acquire images with the METAFLUOR 7.7 software from Molecular Devices. Dual-emission ratio imaging was performed for InPTapp, InPGrp, or InPAkt with a 420DF20 excitation filter, a 455DRLP dichroic mirror, and 473DF24 CFP and 535DF25 YFP emission filters. To image the mCherry tagged phosphatases, a 572DF35 excitation filter, a 594 DRLP dichroic mirror, and a 645DF75 emission filter were used. For all epifluorescence imaging experiments, exposure times of 50-500 ms were used with images being taken once every 30 seconds. Metafluor 7.7 software (Molecular Device) was used to analyze the data. Background correction was conducted using raw fluorescence intensity values in regions with no cells. Yellow-to-cyan (Y/C) emission were calculated using background corrected values and normalized to the time point before drug addition. Time courses plot R/R₀, where R is the biosensor emission ratio at each time point and R0 is the average of the baseline values (before treatment). Ratio changes presented as bar graphs were calculated to identify the maximum normalized emission ratio by taking the average of the maximum three ratio values recorded after treatment. Rₘₐₓ reported in the text was calculated as (Rₘₐₓ−R₀)*100. Plasma membrane or lysosomal regions of interest were drawn to analyze subcellularly targeted reporters. Kinetics were characterized using t_{1/2}, calculated by the time required to reach half of the maximum response amplitude, and the Sustained Activity Metric at 15 minutes after treatment (SAM15), which was calculated by (R₁₅−R₀)/(Rₘₐₓ−R₀), where R15 is the normalized emission ratio 15 minutes after treatment. Baseline correction was performed for LAMP1-InPTapp response to PDGF with pre-treatment of PIK-75 in FIG. 9B to correct for baseline drifting. Linear regression analysis was conducted on the baseline response of the sensor, prior to any treatment. The slope of the fitted line was used to correct for drifting in the curve using the following equation: R=R−(slope*time).

Colocalization and Endocytosis Assay with Live Cell Spinning Disk Confocal Imaging

NIH3T3 cells were incubated with 100 nM of LysoTracker Green or Red in modified Hank's balanced salt solution, including 1×HBSS with 2 g/L glucose, pH 7.4, (10×HBSS, 14065, GIBCO) for 30 minutes before imaging. A Nikon Ti2 Scope (Nikon) equipped with a Wi confocal scanhead (Yokogawa Electric), Dual Prime95B (Photometrics) cameras, LUN-F-XL laser engine (Nikon), Nano-Drive (Mad City Labs), Galvo XY Scanner, Sola Light box (Lumencor), Piezo Z-Stage (Mad City Labs), Bandpass filter cubes (Chroma), and an Apo TIRF 100×1.49 NA objective was used to acquire images with the NIS Elements AR software (Nikon). Laser lines at 488 nm and 561 nm were used to co-image LysoTracker Green or Red and mCherry tagged (red) phosphatases fluorescence emission or Lyso-InPGrp. Pearson's coefficient of correlation between the plasma membrane targeted phosphatases and LysoTracker was calculated using the Coloc2 plug-in on Fiji (Image J).

Serum-starved NIH3T3 cells were incubated with 100 nM of LysoTracker Red in modified Hank's balanced salt solution, including 1×HBSS with 2 g/L glucose, pH 7.4, (10×HBSS, 14065, GIBCO) for 10 minutes before imaging. Using the same confocal setup as the colocalization assay, laser lines at 405 nm and 561 nm were used to co-image ECGreen and LysoTracker Red fluorescence emission. Cells were imaged for at least 5 minutes, with image acquisition every 15 seconds. After imaging 3-4 timepoints with only LysoTracker Red, cells were treated with 2 μL of ECGreen (1:1,000 dilution). Following 1.5 minutes of ECGreen incubation, medium containing ECGreen was replaced with fresh HBSS. Cell imaging was then continued for 2-3 more minutes. Line traces of GFP and RFP fluorescence were generated using Fiji (Image J) to depict fluorescence colocalization of endocytosed ECGreen dye with LysoTracker stained lysosomes as early as 1.5 to 2 minutes after incubation with ECGreen.

### Immunofluorescence Assay

Immunofluorescence was conducted to detect PIP3 colocalization with the lysosomal marker Rab7 upon PDGF stimulation. Cells were plated on glass slides, incubated in serum-free DMEM for 24 hours, and incubated with HBSS for 10 minute at room temperature prior. They were then fixed using 4% paraformaldehyde (PFA) for 20 minutes, then washed with PBS and permeabilized using 0.1% Tween-20 in PBS with 5% BSA for 15 minutes. After permeabilization, blocking was conducted using 5% BSA in PBS for 30 minutes. Primary antibody incubation was conducted overnight at 4° C. using mouse anti-PIP3 and rabbit anti-Rab7 antibodies both diluted 1:50 in 5% BSA in PBS. Secondary antibody incubation was conducted with anti-mouse Alexa Fluor 488 and anti-rabbit Alexa Fluor 555 antibodies diluted 1:20,000 in 5% BSA in PBS at room temperature for 45 minutes. Cells were then mounted using mounting buffer and imaged by spinning disk confocal microscopy as described for the colocalization and endocytosis assays above.

### Structure Prediction and Analysis

The complex structures of the lipid binding domains and pseudoligand were predicted using the AlphaFold 3 algorithm (66) via the AlphaFold server. Specifically, two models corresponding to the biosensors InPGrp and InPTapp were generated. Each model was constructed by inputting one copy of the respective lipid binding domain and its pseudoligand into the AlphaFold server. The pseudosubstrate sequence used for the predictions was GGSVAEEEDDEEEDEDDGGS (SEQ ID NO:57). The resulting models were subsequently visualized and analyzed using UCSF ChimeraX software (67). The published structures, 1FHW (20) and 1EAZ (33), of the respective lipid bound states of Grp1 and Tapp1 were visualized using ChimeraX as well. Lastly, the interactions of the pseudoligand and the lipid binding domains of InPGrp and InPTapp in the presence of respective target lipids were predicted using Chai-1 using its web server (68).

For PIP₃, the following SMILES was used:

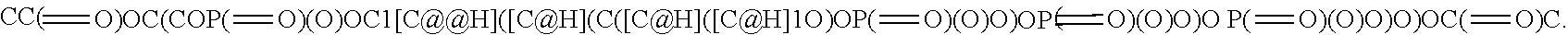

For PI(3,4)P₂, the following SMILES was used:

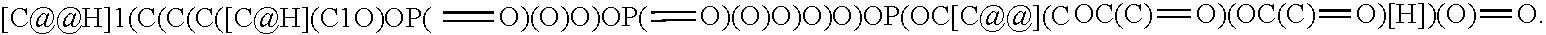

### Expression and Purification of InPGrp

E. coli BL21 (DE3) chemically competent cells were transformed with His-tagged InPGrp in pRSET-B vector. A single colony selected from an LB-Agar plate containing ampicillin (100 μg/ml) was inoculated in 20 mL LB-ampicillin (100 μg/ml) media and cultured overnight at 37° C. This seed culture was then inoculated in 1 L of LB-ampicillin media and cultured at 37° C. with shaking (200 rpm) until reaching an OD600 between 0.6 and 1.0. His-InPGrp expression was induced by adding 500 μM IPTG and cultured for 18 hours at 16° C. with shaking. Cells were harvested by centrifugation (5000×g, 10 min, 4° C.) and directly proceeded to protein purification.

Cell pellets were chilled on ice prior to resuspension in lysis buffer (50 mM Tris, pH 7.4, 300 mM NaCl, 1 mM DTT) containing Complete Protease Inhibitor Cocktail (EDTA-free, 1 tablet/50 ml, Roche). Cells were then lysed by probe sonication at 100% amplitude and ten 30 seconds ON/30 seconds OFF cycles on ice. Cell debris was removed by centrifugation (45000×g for 0.5 hours, 4° C.), and the supernatant was filtered through 0.45 m membrane filter before loaded onto a pre-packed Hispur™ nickel nitrilotriacetic acid resin column (Ni-NTA, 5 ml, GE Healthcare) through a syringe at roughly 1 ml/min at 4° C. The column was then washed thoroughly with washing buffers (lysis buffer containing imidazole) at increasing imidazole concentrations: 0 imidazole, 25 ml; 20 mM imidazole, 50 ml; 70 mM imidazole, 20 ml. His-InPGrp was then eluted with lysis buffer supplemented with a total of 300 mM imidazole. Fractions were analyzed by SDS-PAGE and the pure fractions were pooled, concentrated, and buffer exchanged to 50 mM Tris, pH 7.4, 300 mM NaCl, 10% glycerol using Amicon Ultra-15 centrifugal columns (30-kD cut-off, Millipore).

### Protein-Lipid Interaction Assay Using PIP Strip

The PIP strip (P-6001, Echelon Biosciences) was stored in the dark at 4° C. when not in use and all incubations were conducted in the dark at room temperature. The PIP strip was blotted with 1 μL of purified InPGrp to serve as a positive control for detection and allowed to dry for half an hour. The strip was then blocked for 15 minutes with 3% BSA in PBS. Following blocking, the strip was incubated with 1.25 μg/mL of purified InPGrp protein for 2 hours on a shaker, then incubated with mouse anti-6×Histag antibody (diluted 1:1,000 in 3% BSA in PBS) for 1 hour to detect bound InPGrp. Horseradish peroxidase-conjugated anti-mouse antibody, used at 1:10,000 dilution in 3% BSA in PBS, was incubated for 1 hour. Lastly, the PIP strip was imaged with a chemiluminescent imaging using West Pico Plus chemiluminescent detection reagents (#34580, ThermoFisher Scientific).

### Statistical Analysis

All experiments were independently repeated at least three times and statistical tests were noted in each figure legend. GraphPad Prism 9 was used to analyze data. For comparison of two groups, unpaired t-test with Welch's correction was used. For comparison of three or more groups, ordinary one-way ANOVA followed by Dunnett's test for multiple comparisons was used. Statistical significance was set at p<0.05, wherein * indicates a p-value <0.05, ** indicates a p-value <0.01, *** indicates a p-value <0.001, and **** indicates a p-value <0.0001. n numbers representing the number of cells are indicated in the figure legends, and data are presented as mean±standard error of the mean unless otherwise noted.

### Example 1. InPGrp (Indicator of PIP3 Based on GRP) Specifically Detects Plasma Membrane PIP3 Dynamics

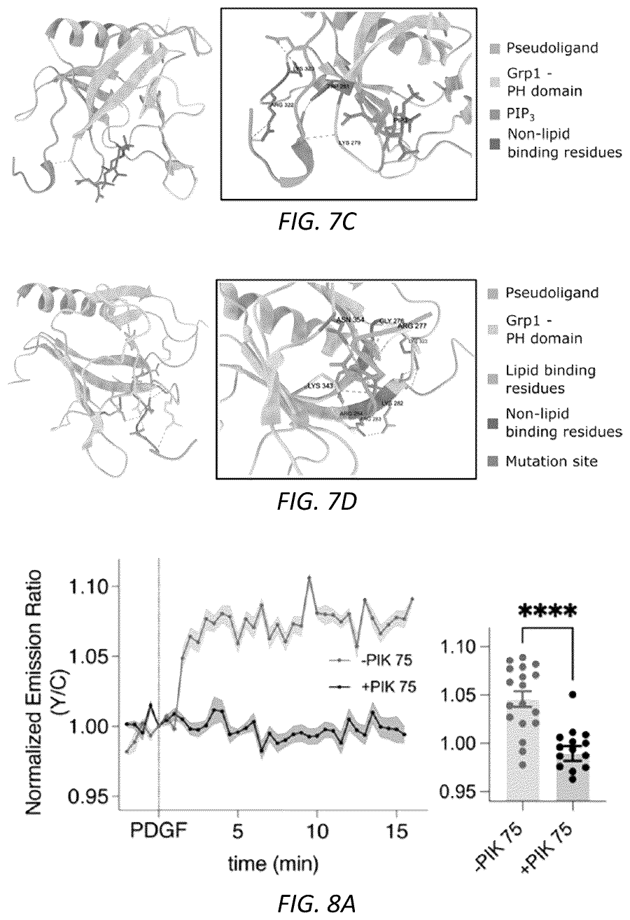

Using the pseudoligand-based design (12), we engineered a FRET-based PIP₃ sensor, InPGrp, and targeted it to the plasma membrane by a C-terminal Kras targeting sequence CAAX (SEQ ID NO:44) (17) (FIG. 1A). The molecular switch of InPGrp consists of a GRP1-PH domain, which specifically binds PIP₃ (18), and a pseudoligand peptide sequence VAEEEDDEEEDEDD (SEQ ID NO:9). The molecular switch is sandwiched in between the cyan and yellow fluorescent proteins, Cerulean (19) and circularly permuted Venus [E172]. PIP₃ interacts with several basic residues in the Grp1 PH domain (FIG. 7A)(20, 21). In the absence of PIP₃, the pseudoligand is predicted to interact with several of the same basic residues in Grp1 (FIG. 7B). Once present, the natural ligand, PIP3, is predicted to outcompete the pseudoligand (FIG. 7C), releasing the pseudoligand from the lipid binding pocket of the PH domain and generating a conformational change that leads to an increase in the yellow to cyan emission ratio (FIG. 1A).

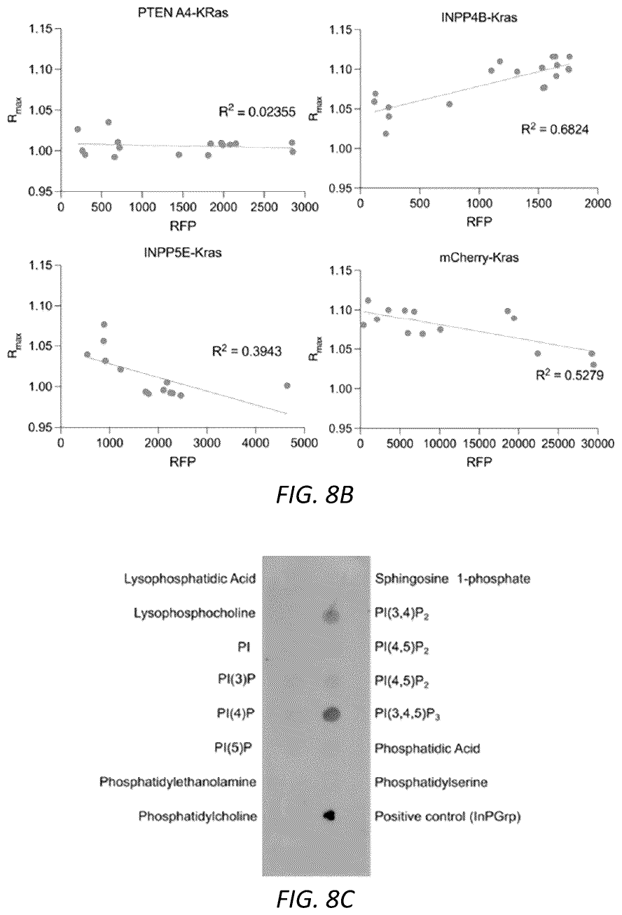

Growth factor stimulation leads to a rapid generation of PIP₃ and PI(3,4)P₂ at the plasma membrane. To detect the generation of PIP₃ at the plasma membrane, we treated serum starved NIH3T3 cells with PDGF and quantified the resulting change in yellow to cyan emission ratio of InPGrp-Kras 15 minutes post-treatment. As shown by the average response curve (FIG. 1B), the increase in emission ratio of InPGrp-Kras following PDGF treatment indicates PIP3 accumulation at the plasma membrane. To confirm that the sensor response is dependent on PIP3 binding, we generated a mutant InPGrp-Kras (K273A), which contains a K273A mutation in the PH domain that abolishes PIP₃ binding (18). This mutant sensor is predicted to retain the interaction between the pseudoligand and the lipid binding domain (FIG. 7D), but is unable to bind PIP₃, thereby serving as a suitable control for nonspecific interactions of the sensor which may contribute to the observed response. As expected, the mutant sensor exhibited no obvious changes in response to PDGF stimulation, indicating InPGrp response specificity for PIP₃. Additionally, we tested whether growth factor stimulated PIP3 accumulation at the plasma membrane was in agreement with canonical class I PI3K signaling (22). When NIH3T3 cells were pretreated with the class IA PI3K inhibitor PIK-75, InPGrp-Kras response to PDGF was abolished (FIG. 8A), suggesting that the PIP3 accumulation observed via InPGrp is dependent on PI3K activity, as expected.

Next, we characterized the signal specificity of InPGrp-Kras for PIP3 compared to similar lipid species. To perturb phosphoinositides (PIs) in a site-specific manner, we targeted lipid phosphatases tagged with red fluorescent protein mCherry to the plasma membrane using the CAAX (SEQ ID NO:44) (Kras) motif (FIG. 1C, domain structures). We used three lipid phosphatases that selectively degrade different phosphoinositides: PTENA4, INPP4B, and INPP5E. PTEN (phosphatase and tensin homologue)-A4 is a constitutively active form of PTEN that incorporates 4 alanine mutations in the C-terminal tail region of the phosphatase (23-25). PTENA4 converts PIP₃ and PI(3,4)P₂ to PI(4,5)P₂ and PI4P, respectively, and INPP5E converts PIP₃ and PI(4,5)P₂ to PI(3,4)P₂ and PI4P (22, 26-29). Consistent with the PIP₃ depletion effects of these two phosphatases, cells co-expressing InPGrp-Kras with PTENA4-Kras or INPP5E-Kras exhibited diminished response to PDGF compared to cells co-expressing the sensor with the vector control mCherry-Kras (FIG. 1D). We observed that increased expression of INPP5E-Kras correlated to lower InPGrp-Kras emission ratio after PDGF stimulation, whereas PTENA4 did not have such a trend (FIG. 8B). Furthermore, INPP4B-Kras, which converts PI(3,4)P₂ to PI3P (30), did not significantly affect InPGrp-Kras response to PDGF compared to the vector control and WT conditions (FIG. 1D). These findings, together with an in vitro lipid-protein interaction assay (FIG. 8C), demonstrate specificity of the sensor for PIP3 over other similar lipids, including PI(3,4)P₂. Overall, we developed InPGrp, a lipid biosensor specifically sensing changes in PIP3 levels. InPGrp was able to detect PDGF induced accumulation of PIP3 at the plasma membrane.

### Example 2. InPTapp (Indicator of PI(3,4)P₂ Based on Tapp) Specifically Detects Plasma Membrane PI(3,4)P₂ Dynamics

InPTapp, the PI(3,4)P₂ biosensor, was engineered using a design like that of InPGrp. The molecular switch incorporates the Tapp1-PH domain, which specifically binds to PI(3,4)P₂, and the same pseudoligand peptide as in InPGrp. These two elements are sandwiched between Cerulean and Citrine fluorescent proteins (14, 31, 32). InPTapp-Kras was targeted to the plasma membrane by fusing a CAAX (SEQ ID NO:44) targeting sequence to the C-terminus (FIG. 2A, domain structure). In a mechanism similar to InPGrp, the pseudoligand is expected to interact with several basic residues in the PH domain of InPTapp in the absence of PI(3,4)P₂ (FIGS. 9A-9B). Once the natural ligand, PI(3,4)P₂, is accumulated, the pseudoligand is predicted to be competed out of the lipid binding pocket of the PH domain (FIG. 9C), generating a conformational change that leads to an increase in the yellow over cyan emission ratio (FIG. 2A).

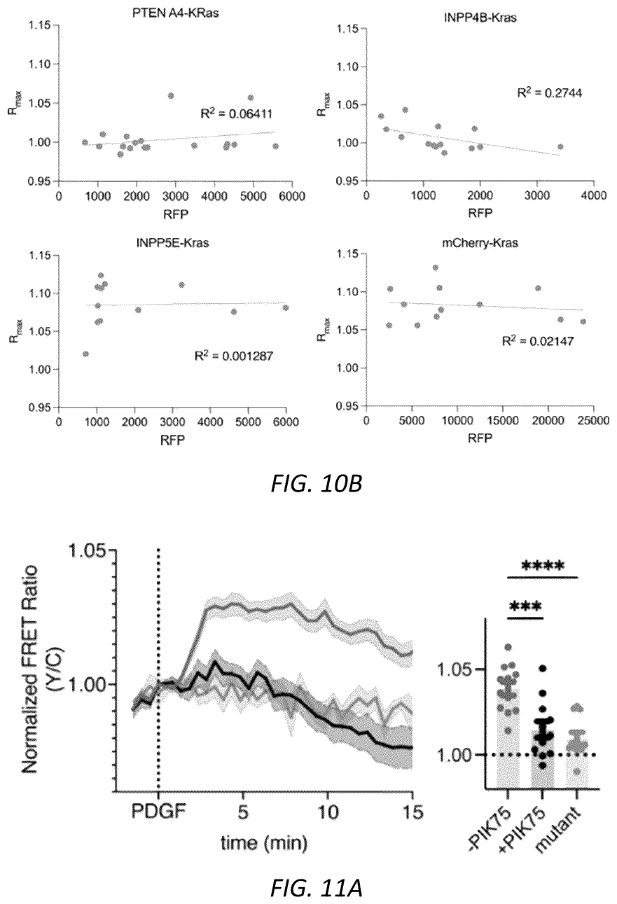

Like PIP₃, PI(3,4)P₂ is generated at the plasma membrane upon activation of PI3K by external stimuli. Upon growth factor treatment, the yellow/cyan emission ratio of InPTapp-Kras increased, indicating accumulation of PI(3,4)P₂ at the plasma membrane in serum starved NIH3T3 cells (FIG. 2B). A mutant version of InPTapp-Kras incorporating an R21 1L mutation in the Tapp1-PH domain (31) was engineered to validate the requirement of lipid binding (FIG. 9D). This mutant InPTapp-Kras (R21 1L) exhibited no obvious changes in response to PDGF stimulation (FIG. 2B). Additionally, we probed whether growth factor stimulated PI(3,4)P₂ accumulation at the plasma membrane relies on the canonical class I PI3K signaling (22). When NIH3T3 cells were pretreated with PIK-75, a PI3K inhibitor, plasma membrane-targeted InPTapp showed no changes in emission ratio upon PDGF treatment (FIG. 10A), indicating PI3K activity is required.

Next, we characterized the signal specificity of InPTapp-Kras for PI(3,4)P₂ in comparison with other lipid species. InPTapp-Kras was co-expressed with PTENA4-Kras, INPP4B-Kras, INPP5E-Kras, or mCherry-Kras in serum starved NIH3T3 cells stimulated with growth factor (FIG. 2C). Representative cell curves demonstrate that both PTENA4 and INPP4B, which convert PI(3,4)P₂ to other lipid species, expressed at the plasma membrane, diminished InPTapp-Kras response to PDGF stimulation (FIG. 2D). Conversely, overexpression of INPP5E-Kras, which only converts PIP₃ to PI(3,4)P₂ and PI4P, or the vector control mCherry-Kras, had no effect on InPTapp-Kras response to PDGF (FIG. 2D). InPTapp-Kras therefore exhibits a signal specifically dependent on PI(3,4)P₂.

### Example 3. Both PIP3 and PI(3,4)P2 Accumulate at the Lysosome

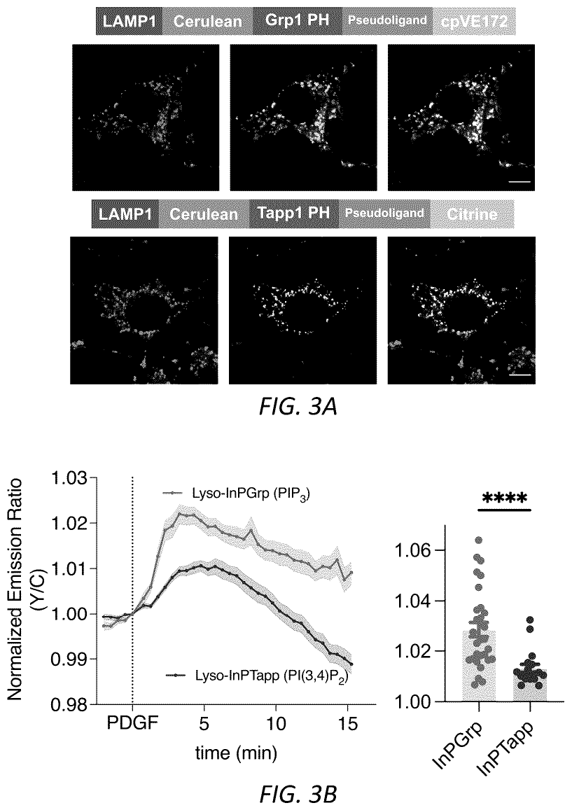

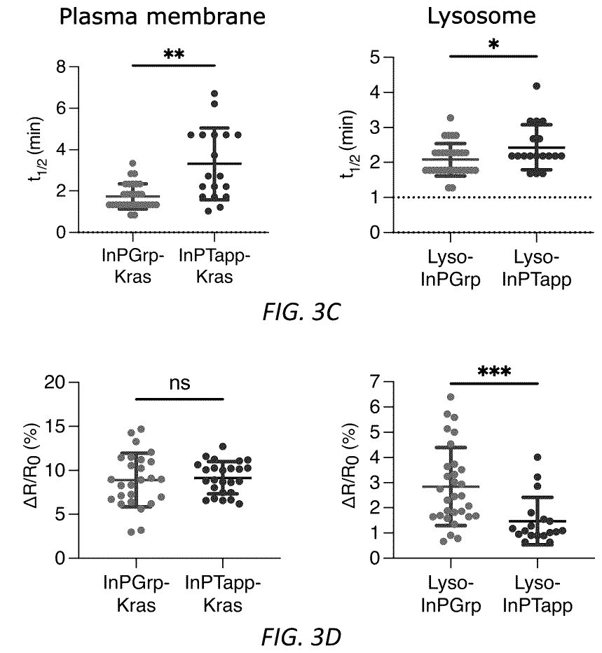

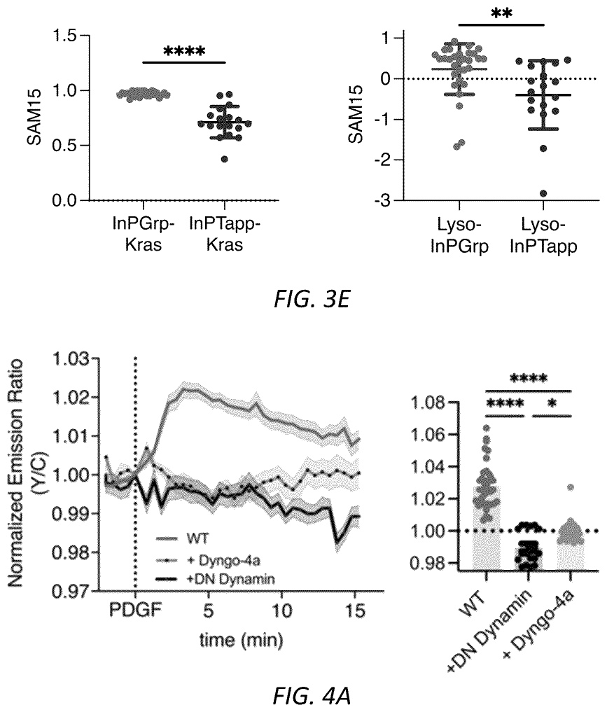

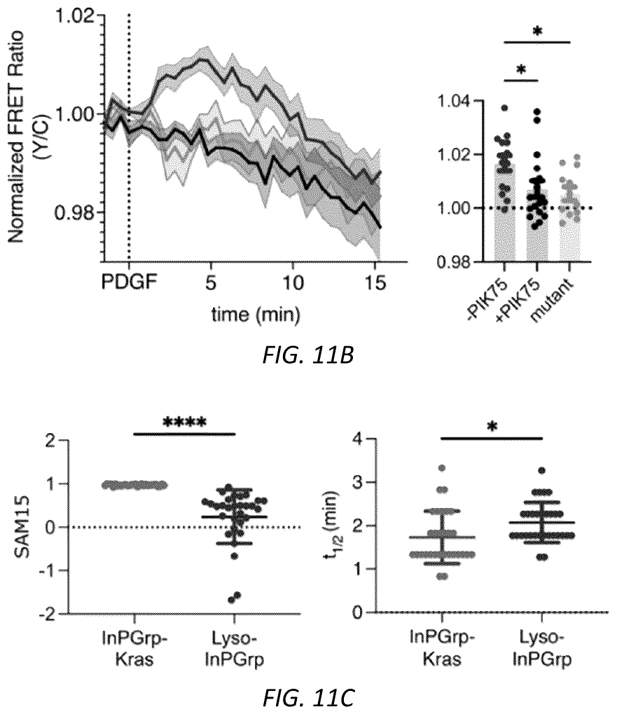

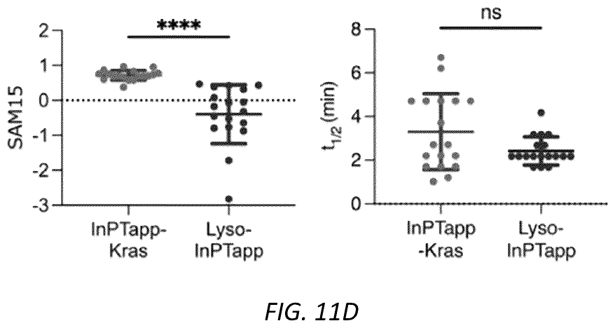

The lysosome is an emerging signaling hub and a major site of mTORC1 signaling, yet the dynamics of PIP₃ and PI(3,4)P₂ accumulation at the lysosome have not been well characterized. Due to the importance of these lipids as signaling molecules that can promote Akt/mTOR signaling, we aimed to investigate which of the 3-PI species (PIP₃, PI(3,4)P₂, or both) accumulate at the lysosomal membrane and how they reach the lysosome. To probe lysosomal accumulation of PIP₃ and PI(3,4)P₂, we targeted InPGrp and InPTapp to the lysosome by fusing the LAMP1 lysosomal targeting motif to the N-terminus of each biosensor. These biosensors exhibited lysosomal localization as indicated by colocalization with the LysoTracker dye (FIG. 3A). Lyso-InPGrp reported PDGF-stimulated PIP₃ accumulation at the lysosome via an increase in Y/C emission ratio in serum staved NIH3T3 cells (FIG. 3B). Lyso-InPTapp demonstrated a small but consistent response to PDGF stimulation, indicating lysosomal accumulation of PI(3,4)P₂ as well (FIG. 3B). Compared to the control, incorporation of the K237A mutation to abolish the phosphoinositide binding or pretreatment of cells with PIK75 abolished lysosomal InPGrp response (FIG. 11A). Similarly, Lyso-InPTapp response to PDGF was diminished by incorporation of the R21 1L mutation or PIK75 pretreatment (FIG. 11B).

To further characterize the accumulation of PIP₃ and PI(3,4)P₂ at the lysosome and the plasma membrane, we compared the mean t_{1/2} (time to half of maximum response) of the InPGrp and InPTapp responses. Mean t_{1/2} of the InPTapp response was longer than that of InPGrp at both the plasma membrane and the lysosome (FIG. 3C), consistent with the previous notion that PI(3,4)P₂ is likely generated by dephosphorylation of PIP₃ (34, 35). Furthermore, t_{1/2} of InPGrp at the lysosome is longer than at the plasma membrane (FIG. 11C), hinting at the possibility that the plasma membrane pool of PIP₃ is a precursor or requirement for lysosomal PIP₃ accumulation following PDGF stimulation.

Another major parameter is the response amplitude. InPGrp and InPTapp showed comparable PDGF-induced responses at the plasma membrane (FIG. 3D), suggesting the dynamic range of these two biosensors is comparable. However, the lysosomal response amplitude of InPGrp was significantly higher than that of InPTapp, revealing that lysosomal accumulation of PIP₃ is greater than that of PI(3,4)P₂ (FIG. 3D).

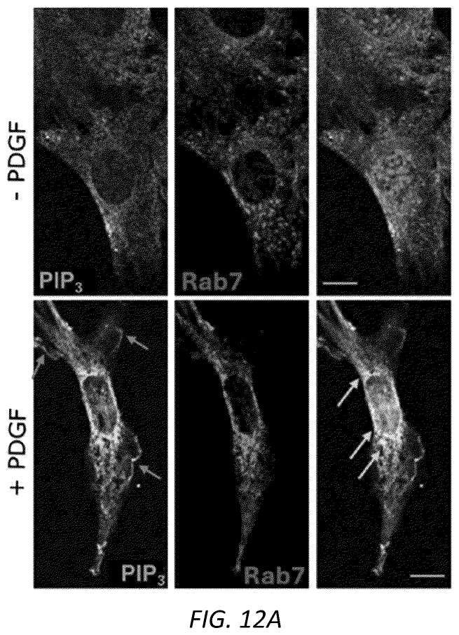

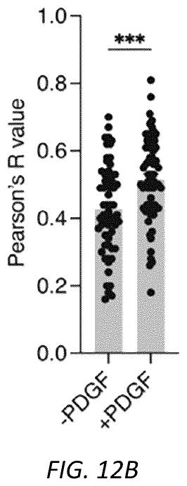

The Sustainable Activity Metric 15 (SAM 15) measures the sustainability of the biosensor response obtained by dividing the magnitude of the endpoint response (at 15 minutes) by the maximum response (36). At both locations, InPGrp exhibits a higher SAM15 value than InPTapp, indicating that PIP₃ dynamics are more sustained than that of PI(3,4)P₂(FIG. 3E). The transience of InPTapp response may indicate reduced conversion of PIP₃ to PI(3,4)P₂ or increased conversion of PI(3,4)P₂ to other lipids over time at both the plasma membrane and lysosomes. The sustainability of InPGrp response, however, suggests that PIP₃ accumulation dominates over PI(3,4)P₂, with increasing preference over time after PDGF treatment. Immunostaining experiments using an anti-PIP₃ antibody further validated the lysosomal accumulation of PIP₃(FIG. 12). Overall, these data reveal preferential accumulation of PIP₃ at lysosomes.

### Example 4. Accumulation of PIP3 and PI(3,4)P2 at the Lysosome is Dependent on Endocytosis

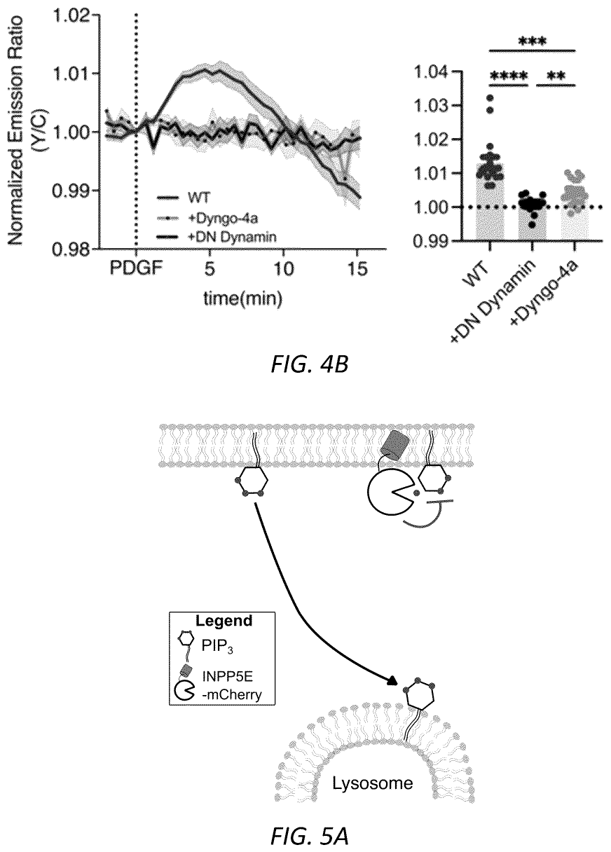

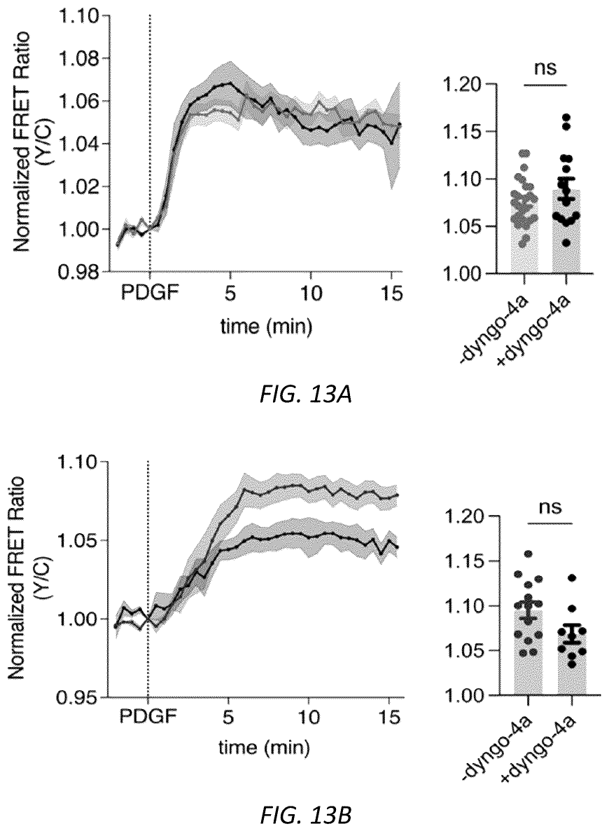

The systematic comparison of PIP₃ and PI(3,4)P₂ at the plasma membrane and lysosome reveals important insights into the dynamics of subcellular lipid accumulation but leaves the question of how these lipids accumulate at lysosomes unanswered. Lysosomal lipid accumulation follows plasma membrane lipid generation, based on the longer t_{1/2} of InPGrp at the lysosome compared to the plasma membrane. We therefore set out to determine whether there is a plasma membrane-to-lysosome trafficking mechanism that is required for lysosomal lipid accumulation. A common mechanism of protein and lipid trafficking to lysosomes is via the endocytic pathway (37). To test the role of dynamin-dependent endocytosis in lysosomal accumulation of PIP₃ or PI(3,4)P₂, we pretreated cells expressing Lyso-InPGrp or Lyso-InPTapp with Dyngo-4a, a pharmacological inhibitor of dynamin. Lyso-InPGrp response to PDGF was abolished upon pretreatment with Dyngo-4a, indicating that dynamin-dependent endocytosis is required for lysosomal PIP3 accumulation (FIG. 4A). Dyngo-4a pretreatment abolished growth factor stimulated Lyso-InPTapp response as well (FIG. 4B), indicating that lysosomal accumulation of PI(3,4)P₂ also requires dynamin-dependent endocytosis. On the other hand, Dyngo-4a pretreatment did not significantly affect the PDGF-induced responses of plasma membrane targeted InPGrp and InPTapp (FIG. 13).

The effect of inhibiting dynamin-mediated endocytosis on lysosomal 3-PI accumulation was validated using an alternative method of inhibition: expression of dominant negative dynamin-2 (DN-dynamin, harboring the K44A mutation) (38). Lyso-InPGrp and Lyso-InPTapp responses to PDGF were abolished in cells expressing DN-dynamin (FIG. 4), confirming the dependence of lysosomal PIP₃ and PI(3,4)P₂ accumulation on dynamin-dependent endocytosis.

### Example 5. PIP3 is Generated at the Plasma Membrane and Internalized to the Lysosome

The dependence of lysosomal PIP₃ accumulation on endocytosis could be explained by two possible mechanisms: (1) an essential signaling component(s), such as the receptor or a lipid kinase, could be internalized, leading to the accumulation of PIP₃ at the lysosome; or (2) PIP₃ generated at the plasma membrane is itself internalized via endocytosis, and trafficked to lysosomes.

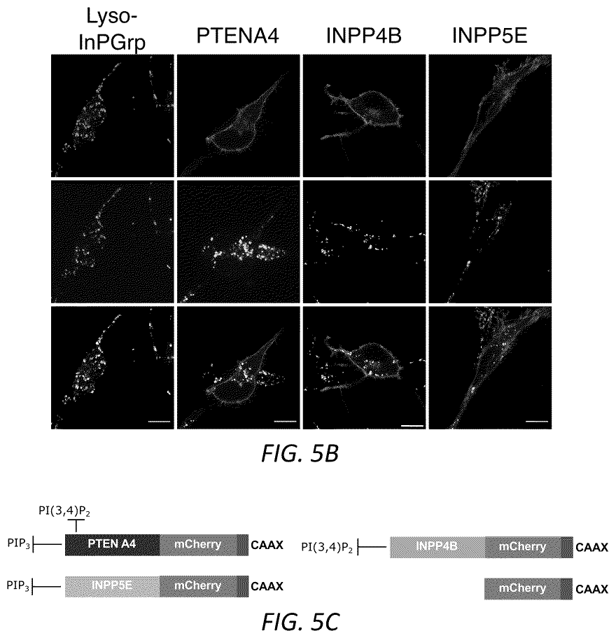

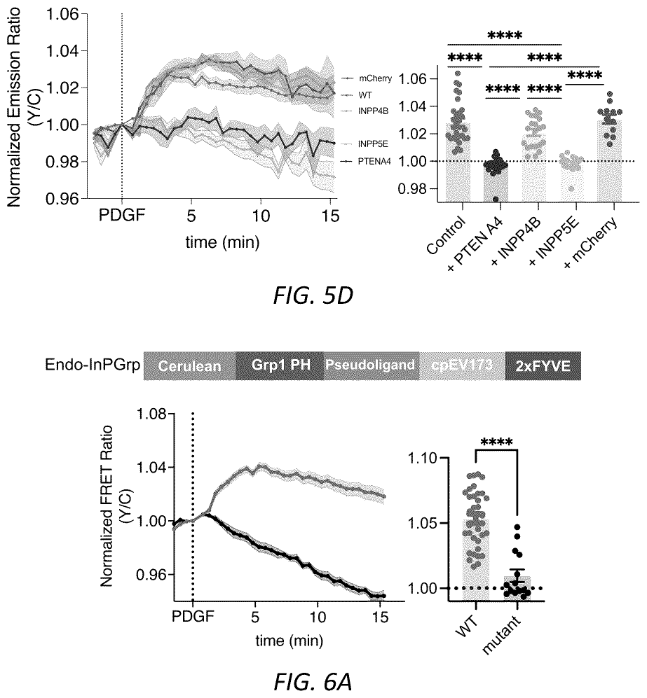

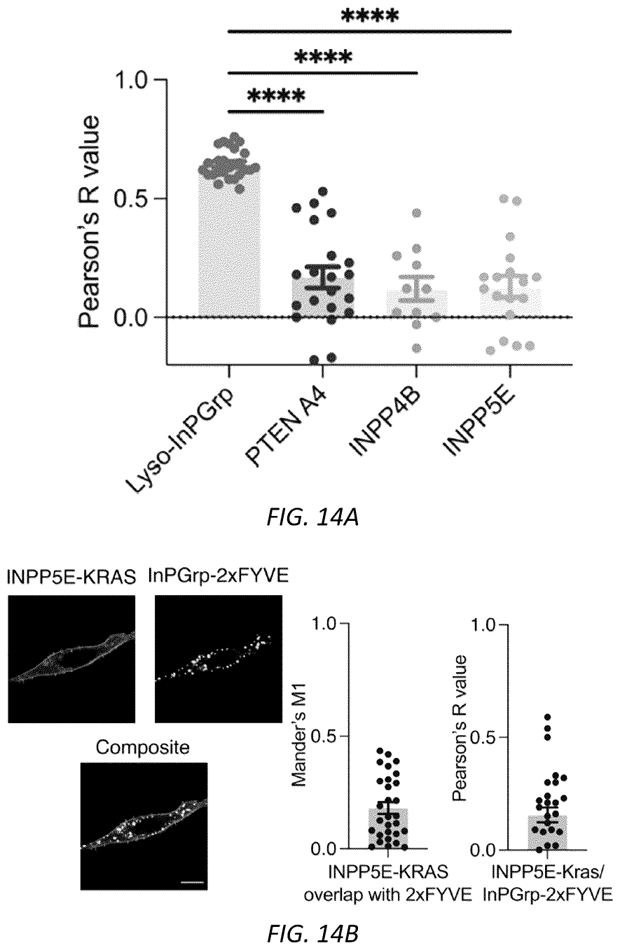

To directly test the latter model of PIP₃ internalization, we designed a set of experiments to utilize plasma membrane-targeted phosphatases to deplete the plasma membrane pool of PIP₃ and, at the same time, visualize lysosomal PIP₃ dynamics using InPGrp (FIG. 5A). If the model that PIP₃ is endocytosed and trafficked to lysosomes was correct, lysosomal PIP₃ dynamics would be dependent on its plasma membrane counterpart. Thus, overexpression of INPP5E-Kras or PTENA4-Kras at the plasma membrane would perturb lysosomal accumulation of PIP₃ by depleting the plasma membrane source of this lipid. To establish the experimental conditions, we first validated the specificity of plasma membrane targeting for these phosphatases. INPP4B-Kras, PTENA4-Kras, INPP5E-Kras, and mCherry-Kras localized to the periphery of cells, showing little colocalization with lysosomal or endosomal markers (FIGS. 5B and 14A-14B).

Next, we investigated whether plasma membrane targeted phosphatases would perturb lysosomal PIP3 accumulation induced by growth factor treatment (FIGS. 5C-5D). PDGF-stimulated Lyso-InPGrp responses were analyzed and compared with or without co-expression of plasma membrane-targeted lipid phosphatases, PTENA4, INPP4B, and INPP5E (FIG. 5D). Both plasma membrane targeted PTENA4 and INPP5E, which can convert PIP₃ to other lipids, abolished PIP₃ accumulation at the lysosome post PDGF stimulation. Conversely, overexpression of INPP4B-Kras or the vector control mCherry-Kras had no effect on PDGF stimulated lysosomal PIP3 accumulation (FIG. 5D). These data suggest that plasma membrane PIP₃ serves as the source of PIP₃ at the lysosome.

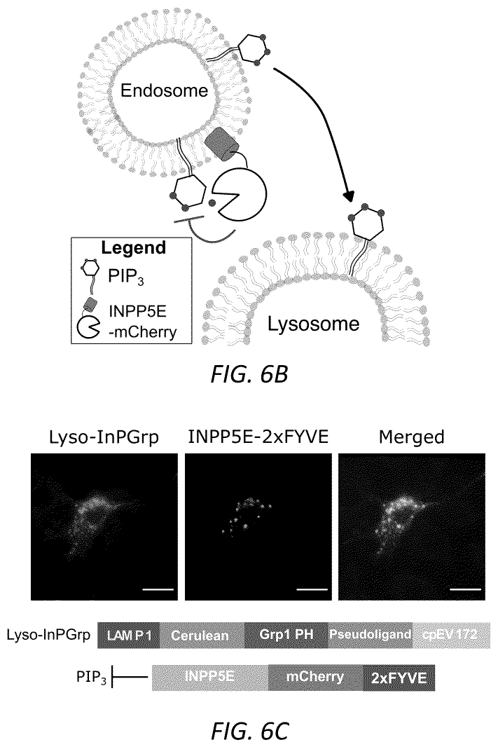

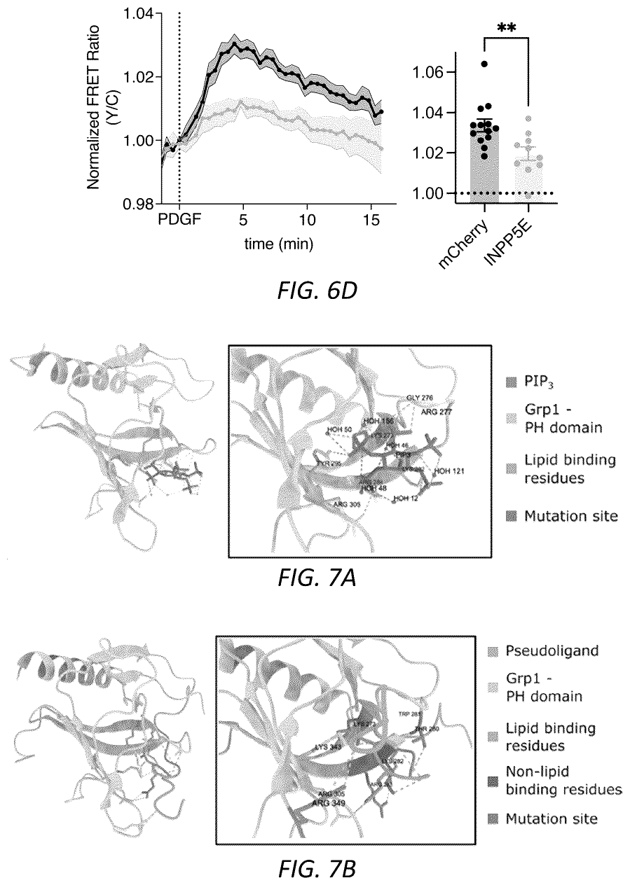

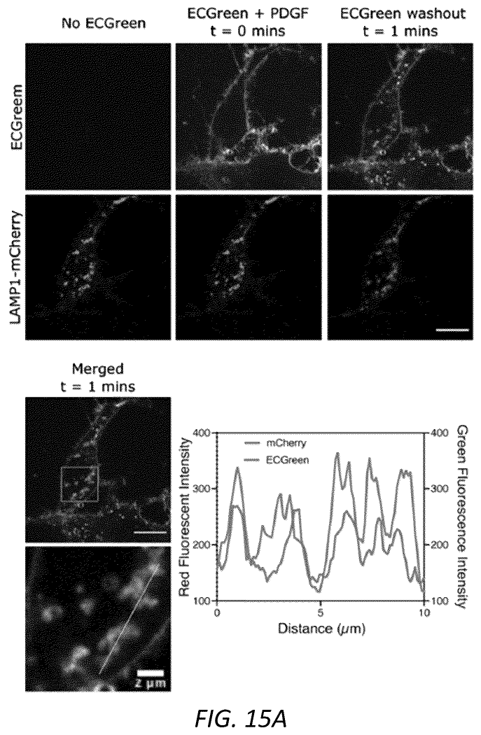

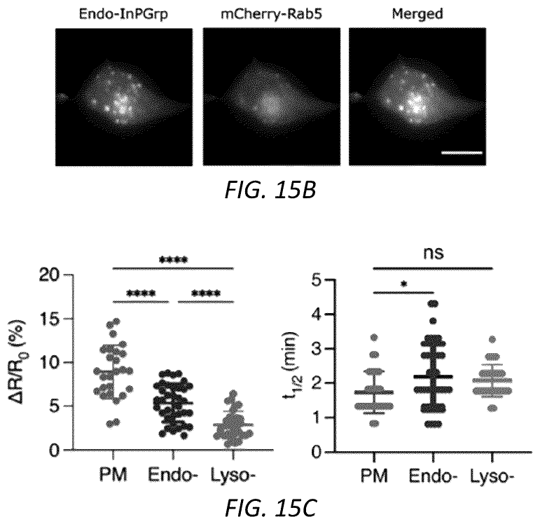

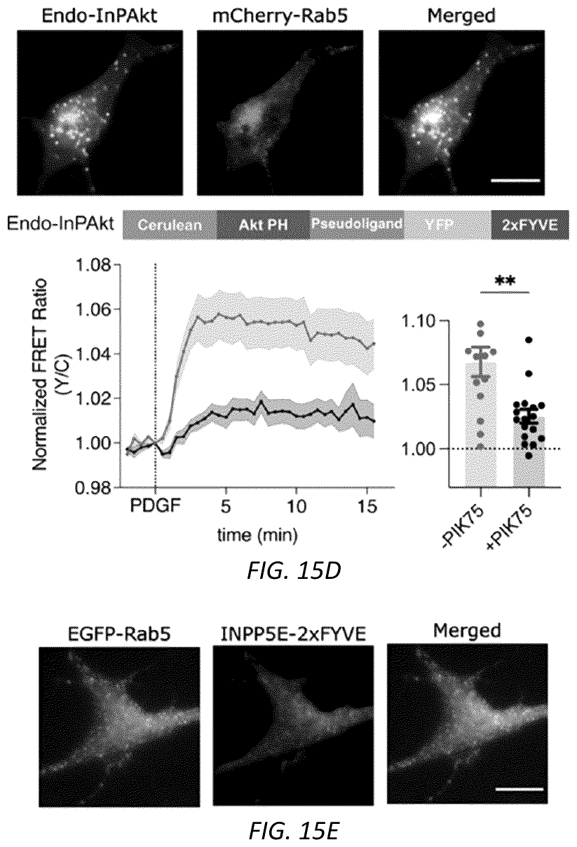

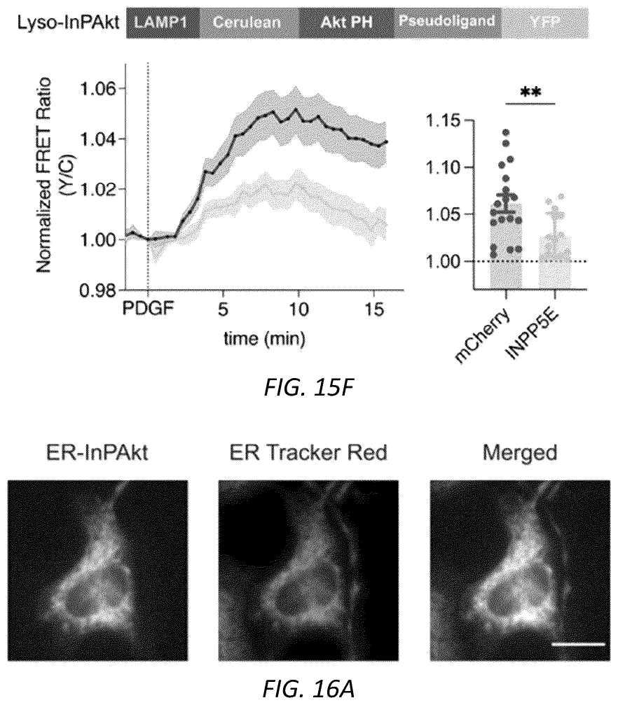

Since inhibition of dynamin-dependent endocytosis abolished lysosomal lipid accumulation, we hypothesized that endocytosis is a key step of PIP3 trafficking to lysosomes. Given the rapid kinetics of lysosomal InPGrp, we aimed to visualize endocytosis in real-time and test if endocytosis and vesicular trafficking to lysosomes can be observed on a time scale in agreement with our biosensor response times. Using ECGreen, a fluorescent dye designed to monitor endocytosis and visualize endocytic vesicles (39), we observed that endocytic vesicles appeared rapidly, within 1 to 2 min of simultaneous PDGF and ECGreen addition, and exhibited proximity or colocalization with lysosomes (FIG. 15A). This observation pointed to a possible route of rapid PIP3 trafficking which involves internalization to endosomes followed by vesicle-based trafficking to the lysosome. We therefore tested the presence of PIP3 at endosomes, given the previous finding that PI(3,4)P₂, but not PIP₃, localizes to endosomes (7). We generated endosome targeted InPGrp (Endo-InPGrp) using the tandem FYVE (SEQ ID NO:58) (2×FYVE) endosomal localization tag (40) to detect PIP₃ at endosomes (FIG. 15B). Following PDGF stimulation of serum starved NIH3T3 cells, we observed significant accumulation of PIP₃ compared to the mutant endo-InPGrp sensor (FIG. 6A), which showed slower kinetics than the plasma membrane response (FIG. 15C). We also utilized InPAkt to serve as an alternative strategy for sensing lysosomal PIP₃ and confirm findings obtained with InPGrp. InPAkt targeted to endosomes, Endo-InPAkt, also detected 3-PI accumulation at endosomes following PDGF stimulation, which was abolished by pretreatment with the PI3K inhibitor, PIK75 (FIG. 15D). All together, these data indicate the presence of PIP₃ at endosomes and suggest that it could be a source for lysosomal accumulation of PIP₃.

If endocytic trafficking of the lipid itself is required for PIP₃ to reach the lysosome, perturbing endosomal PIP₃ should also affect lysosomal PIP₃. We therefore targeted INPP5E to endosomes to test its effect on lysosomal lipid accumulation (FIGS. 6B-6C and 15E). The Lyso-InPGrp response was significantly inhibited by co-expression of endosome-targeted INPP5E compared to endosome-targeted mCherry expression (FIG. 6D). A similar response was observed using Lyso-InPAkt. Compared to the PDGF-induced response of Lyso-InPAkt, endosomal INPP5E expression, which targets PIP₃, significantly diminishes the Lyso-InPAkt response to PDGF (FIG. 15F). Thus, these data suggest that PIP₃ travels to the lysosome from the plasma membrane via endocytosis.

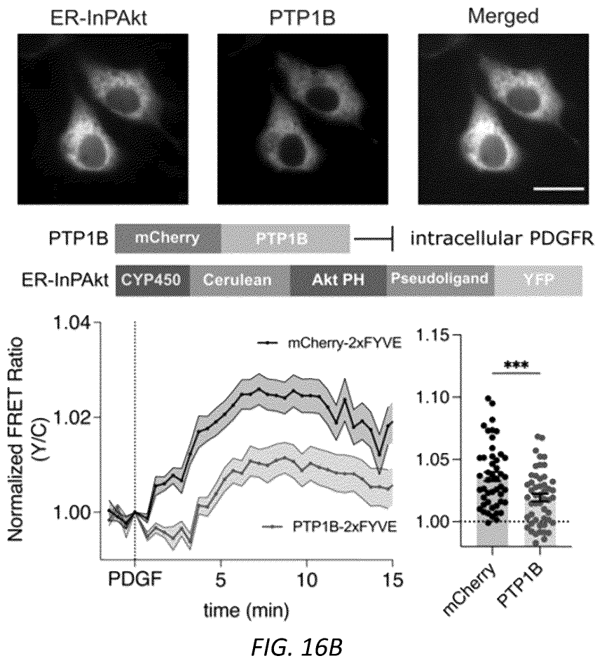

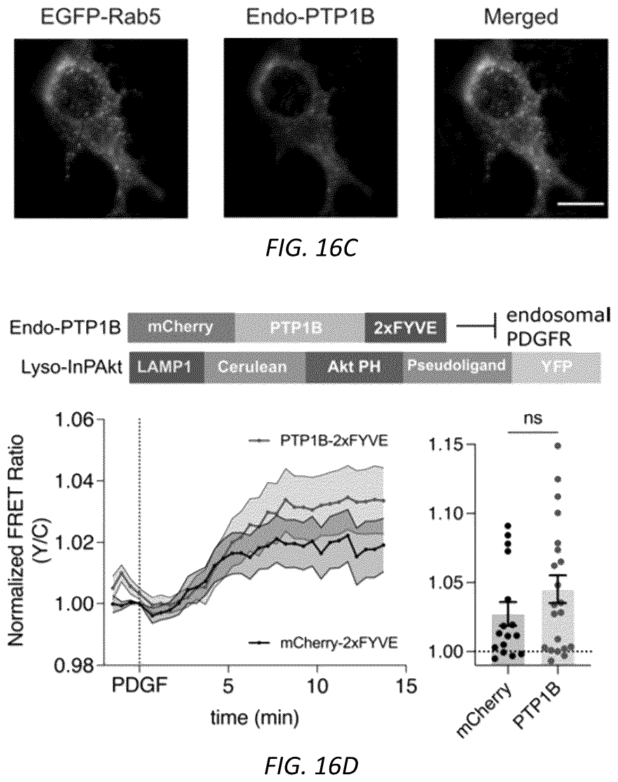

A previous study reported that internalized PDGF receptor (PDGFR) was responsible for the endomembrane accumulation of PIP3 (8). To eliminate the possibility that internalized PDGFR contributes to the lysosomal accumulation of PIP₃, we targeted the PDGFR phosphatase protein tyrosine phosphatase-1B (PTPlB) to the endosome (FIG. 16). If internalized PDGFR generates endosomal PIP₃ that traffics to the lysosome, then endosomal PTP1B should abolish lysosomal PIP₃ accumulation. Although PTP1B expression inhibited accumulation of PIP₃ at the endoplasmic reticulum, which is reported to depend on internalized PDGFR (8) (FIGS. 16A-16B), we found that lysosomal InPGrp response to PDGF was not affected by endosomal PTP1B (FIGS. 16C-16D). Consequently, lysosomal PIP₃ accumulation is not dependent on internalized PDGFR.

In summary, we show that PDGF-induced lysosomal accumulation of PIP₃ is dependent on the plasma membrane pool of PIP₃ and rapid endocytosis, but not dependent on internalized PDGFR. These data suggest a model that PIP₃ is first generated at the plasma membrane and then trafficked to the lysosome via dynamin-mediated endocytosis.

### REFERENCES

1. C. Janetopoulos, P. Devreotes, Phosphoinositide signaling plays a key role in cytokinesis. J Cell Biol 174, 485-490 (2006).
2. Y. Posor, W. Jang, V. Haucke, Phosphoinositides as membrane organizers. Nat Rev Mol Cell Biol 23, 797-816 (2022).
3. O. Idevall-Hagren, E. J. Dickson, B. Hille, D. K. Toomre, P. De Camilli, Optogenetic control of phosphoinositide metabolism. Proc Natl Acad Sci USA 109, E2316-2323 (2012).
4. L. Palamiuc, A. Ravi, B. M. Emerling, Phosphoinositides in autophagy: current roles and future insights. FEBS J287, 222-238 (2020).
5. R. Katso, et al., Cellular function of phosphoinositide 3-kinases: implications for development, homeostasis, and cancer. Annu Rev CellD ev Biol 17, 615-675 (2001).
6. E. C. Lien, C. C. Dibble, A. Toker, PI3K signaling in cancer: beyond AKT. Curr Opin Cell Biol 45, 62-71 (2017).
7. S.-L. Liu, et al., Quantitative Lipid Imaging Reveals a New Signaling Function of Phosphatidylinositol-3,4-Bisphophate: Isoform- and Site-Specific Activation of Akt. Mol Cell 71, 1092-1104.e5 (2018).
8. M. Sato, Y. Ueda, T. Takagi, Y. Umezawa, Production of PtdInsP3 at endomembranes is triggered by receptor endocytosis. Nat Cell Biol 5, 1016-1022 (2003).
9. F. Mazloumi Gavgani, et al., Nuclear Phosphatidylinositol 3,4,5-Trisphosphate Interactome Uncovers an Enrichment in Nucleolar Proteins. Mol Cell Proteomics 20, 100102 (2021).
10. M. Chen, et al., A p53-phosphoinositide signalosome regulates nuclear AKT activation. Nat Cell Biol 24, 1099-1113 (2022).
11. A. J. Valvezan, B. D. Manning, Molecular logic of mTORC1 signalling as a metabolic rheostat. Nat Metab 1, 321-333 (2019).
12. B. Ananthanarayanan, Q. Ni, J. Zhang, Signal propagation from membrane messengers to nuclear effectors revealed by reporters of phosphoinositide dynamics and Akt activity. Proceedings of the National Academy of Sciences 102, 15081-15086 (2005).
13. M. Chen, T. Sun, Y. Zhong, X. Zhou, J. Zhang, A Highly Sensitive Fluorescent Akt Biosensor Reveals Lysosome-Selective Regulation of Lipid Second Messengers and Kinase Activity. ACS Cent Sci 7, 2009-2020 (2021).
14. B. D. Goulden, et al., A high-avidity biosensor reveals plasma membrane PI(3,4)P2 is predominantly a class I PI3K signaling product. J Cell Biol 218, 1066-1079 (2019).
15. T. P. Levine, S. Munro, The pleckstrin homology domain of oxysterol-binding protein recognises a determinant specific to Golgi membranes. Curr Biol 8, 729-739 (1998).
16. H. Yoshizaki, N. Mochizuki, Y. Gotoh, M. Matsuda, Akt-PDK1 complex mediates epidermal growth factor-induced membrane protrusion through Ral activation. Mol Biol Cell 18, 119-128 (2007).
17. J. F. Hancock, K. Cadwallader, H. Paterson, C. J. Marshall, A CAAX or a CAAL motif and a second signal are sufficient for plasma membrane targeting of ras proteins. EMBO J 10, 4033-4039 (1991).
18. F. B. Naughton, A. C. Kalli, M. S. P. Sansom, Association of Peripheral Membrane Proteins with Membranes: Free Energy of Binding of GRP1 PH Domain with Phosphatidylinositol Phosphate-Containing Model Bilayers. J. Phys. Chem. Lett. 7, 1219-1224 (2016).
19. M. A. Rizzo, G. H. Springer, B. Granada, D. W. Piston, An improved cyan fluorescent protein variant useful for FRET. Nat Biotechnol 22, 445-449 (2004).
20. K. M. Ferguson, et al., Structural basis for discrimination of 3-phosphoinositides by pleckstrin homology domains. Mol Cell 6, 373-384 (2000).
21. C.-L. Lai, et al., Molecular Mechanism of Membrane Binding of the GRP1 PH Domain. JMolBiol 425, 3073-3090 (2013).
22. D. A. Fruman, et al., The PI3K pathway in human disease. Cell 170, 605-635 (2017).
23. F. Vazquez, S. Ramaswamy, N. Nakamura, W. R. Sellers, Phosphorylation of the PTEN tail regulates protein stability and function. Mol Cell Biol 20, 5010-5018 (2000).
24. M. Rahdar, et al., A phosphorylation-dependent intramolecular interaction regulates the membrane association and activity of the tumor suppressor PTEN. Proc Natl Acad Sci USA 106, 480-485 (2009).
25. A. H. Ross, A. Gericke, Phosphorylation keeps PTEN phosphatase closed for business. Proc Natl Acad Sci USA 106, 1297-1298 (2009).
26. B. Vanhaesebroeck, L. Stephens, P. Hawkins, PI3K signalling: the path to discovery and understanding. Nat Rev Mol Cell Biol 13, 195-203 (2012).
27. M. Malek, et al., PTEN Regulates PI(3,4)P2 Signaling Downstream of Class I PI3K. Mol Cell 68, 566-580.e10 (2017).
28. T. Asano, Y. Mochizuki, K. Matsumoto, T. Takenawa, T. Endo, Pharbin, a novel inositol polyphosphate 5-phosphatase, induces dendritic appearances in fibroblasts. Biochem Biophys Res Commun 261, 188-195 (1999).
29. A. M. Kong, et al., Cloning and characterization of a 72-kDa inositol-polyphosphate 5-phosphatase localized to the Golgi network. J Biol Chem 275, 24052-24064 (2000).
30. C. Gewinner, et al., Evidence that inositol polyphosphate 4-phosphatase type II is a tumor suppressor that inhibits PI3K signaling. Cancer Cell 16, 115-125 (2009).
31. A. J. Marshall, A. K. Krahn, K. Ma, V. Duronio, S. Hou, TAPP1 and TAPP2 are targets of phosphatidylinositol 3-kinase signaling in B cells: sustained plasma membrane recruitment triggered by the B-cell antigen receptor. Mol Cell Biol 22, 5479-5491 (2002).
32. N. Ariotti, et al., Modular Detection of GFP-Labeled Proteins for Rapid Screening by Electron Microscopy in Cells and Organisms. Dev Cell 35, 513-525 (2015).
33. C. C. Thomas, S. Dowler, M. Deak, D. R. Alessi, D. M. van Aalten, Crystal structure of the phosphatidylinositol 3,4-bisphosphate-binding pleckstrin homology (PH) domain of tandem PH-domain-containing protein 1 (TAPP1): molecular basis of lipid specificity. Biochem J 358, 287-294 (2001).
34. L. R. Stephens, K. T. Hughes, R. F. Irvine, Pathway of phosphatidylinositol(3,4,5)-trisphosphate synthesis in activated neutrophils.

### Nature 351, 33-39 (1991).

35. P. T. Hawkins, T. R. Jackson, L. R. Stephens, Platelet-derived growth factor stimulates synthesis of PtdIns(3,4,5)P3 by activating a PtdIns(4,5)P2 3-OH kinase. Nature 358, 157-159 (1992).
36. J. Keyes, et al., Signaling diversity enabled by Rapl-regulated plasma membrane ERK with distinct temporal dynamics. eLife 9, e57410 (2020).
37. P. Saftig, J. Klumperman, Lysosome biogenesis and lysosomal membrane proteins: trafficking meets function. Nat Rev Mol Cell Biol 10, 623-635 (2009).
38. H. Damke, T. Baba, D. E. Warnock, S. L. Schmid, Induction of mutant dynamin specifically blocks endocytic coated vesicle formation. J Cell Biol 127, 915-934 (1994).
39. K. Qiu, et al., De Novo Design of A Membrane-Anchored Probe for Multidimensional Quantification of Endocytic Dynamics. Adv Healthc Mater 11, e2102185 (2022).
40. A. Hayakawa, et al., Structural basis for endosomal targeting by FYVE domains. J Biol Chem 279, 5958-5966 (2004).
41. R. Weeks, X. Zhou, T. L. Yuan, J. Zhang, Fluorescent Biosensor for Measuring Ras Activity in Living Cells. J. Am. Chem. Soc. 144, 17432-17440 (2022).
42. A. Romin-Fernindez, et al., The phospholipid PI(3,4)P2 is an apical identity determinant. Nat Commun 9, 5041 (2018).
43. S. D. Pauls, A. J. Marshall, Regulation of immune cell signaling by SHIP1: A phosphatase, scaffold protein, and potential therapeutic target. Eur J Immunol 47, 932-945 (2017).
44. S. J. Rodgers, et al., INPP4B promotes PI3Kα-dependent late endosome formation and Wnt/0-catenin signaling in breast cancer. Nat Commun 12, 3140 (2021).
45. W. S. Park, et al., Comprehensive Identification of PIP3-Regulated PH Domains from C. elegans to H. sapiens by Model Prediction and Live Imaging. Mol Cell 30, 381-392 (2008).
46. G. Servant, et al., Polarization of chemoattractant receptor signaling during neutrophil chemotaxis. Science 287, 1037-1040 (2000).
47. M. Iijima, P. Devreotes, Tumor suppressor PTEN mediates sensing of chemoattractant gradients. Cell 109, 599-610 (2002).
48. S. Funamoto, R. Meili, S. Lee, L. Parry, R. A. Firtel, Spatial and temporal regulation of 3-phosphoinositides by PI 3-kinase and PTEN mediates chemotaxis. Cell 109, 611-623 (2002).
49. F. I. Comer, C. A. Parent, PI 3-kinases and PTEN: how opposites chemoattract. Cell 109, 541-544 (2002).
50. S. S. P. Nadiminti, M. Kamak, S. P. Koushika, Tied up: Does altering phosphoinositide-mediated membrane trafficking influence neurodegenerative disease phenotypes? J Genet 97, 753-771 (2018).
51. T. Bohnacker, et al., PI3K7 Adaptor Subunits Define Coupling to Degranulation and Cell Motility by Distinct PtdIns(3,4,5)P3 Pools in Mast Cells. Science Signaling 2, ra27-ra27 (2009).
52. V. I. Korolchuk, et al., Lysosomal positioning coordinates cellular nutrient responses. Nat CellBiol 13, 453-460 (2011).
53. R. Jia, J. S. Bonifacino, Lysosome Positioning Influences mTORC2 and AKT Signaling. Mol Cell 75, 26-38.e3 (2019).
54. S. Sposini, et al., Imaging endocytic vesicle formation at high spatial and temporal resolutions with the pulsed-pH protocol. Nat Protoc 15, 3088-3104 (2020).
55. T. J. Nawara, et al., Imaging vesicle formation dynamics supports the flexible model of clathrin-mediated endocytosis. Nat Commun 13, 1732 (2022).
56. J. B. Doyon, et al., Rapid and efficient clathrin-mediated endocytosis revealed in genome-edited mammalian cells. Nat Cell Biol 13, 331-337 (2011).
57. M. Ecker, G. M. I. Redpath, P. R. Nicovich, J. Rossy, Quantitative visualization of endocytic trafficking through photoactivation of fluorescent proteins. Mol Biol Cell 32, 892-902 (2021).
58. N. L. Chanaday, E. T. Kavalali, Optical detection of three modes of endocytosis at hippocampal synapses. eLife 7, e36097 (2018).
59. T. Soykan, et al., Synaptic Vesicle Endocytosis Occurs on Multiple Timescales and Is Mediated by Formin-Dependent Actin Assembly. Neuron 93, 854-866.e4 (2017).
60. G. T. Saffi, et al., Inhibition of lipid kinase PIKfyve reveals a role for phosphatase Inpp4b in the regulation of PI(3)P-mediated lysosome dynamics through VPS34 activity. Journal of Biological Chemistry 298, 102187 (2022).
61. P. X. Song, et al., Septin 9 and phosphoinositides regulate lysosome localization and their association with lipid droplets. iScience 25, 104288 (2022).
62. J. X. Tan, T. Finkel, A phosphoinositide signalling pathway mediates rapid lysosomal repair. Nature 609, 815-821 (2022).
63. E. J. Dickson, Phosphoinositide transport and metabolism at membrane contact sites. Biochimica et Biophysica Acta (BBA)—Molecular and Cell Biology of Lipids 1867, 159107 (2022).
64. M. Boutry, P. K. Kim, ORP1L mediated PI(4)P signaling at ER-lysosome-mitochondrion three-way contact contributes to mitochondrial division. Nat Commun 12, 5354 (2021).
65. Q. Ma, et al., Mitochondrial PIP3-binding protein FUNDC2 supports platelet survival via AKT signaling pathway. Cell Death Differ 26, 321-331 (2019).
66. J. Abramson, et al., Accurate structure prediction of biomolecular interactions with AlphaFold 3. Nature 1-3 (2024). https://doi.org/10.1038/s41586-024-07487-w.
67. E. F. Pettersen, et al., UCSF Chimera-A visualization system for exploratory research and analysis. Journal of Computational Chemistry 25, 1605-1612 (2004).
68. C. Discovery, et al., Chai-1: Decoding the molecular interactions of life. [Preprint](2024). Available at: https://www.biorxiv.org/content/10.1101/2024.10.10.615955v2 [Accessed 7 Nov. 2024].

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A biosensor polypeptide comprising:
a first linker;
a lipid binding domain comprising the amino acid sequence set forth in SEQ ID NO:1;
a second linker;
a pseudoligand comprising the amino acid sequence set forth in SEQ ID NO:9; and
a third linker.

2. The biosensor polypeptide of claim 1, wherein said biosensor polypeptide comprises, in order from amino terminus (N-terminus) to carboxy terminus (C-terminus):
said first linker;
said lipid binding domain comprising the amino acid sequence set forth in SEQ ID NO:1;
said second linker;
said pseudoligand comprising the amino acid sequence set forth in SEQ ID NO:9; and
said third linker.

3. The biosensor polypeptide of claim 1 or claim 2, wherein the N-terminus of said lipid binding domain is attached to the C-terminus of said first linker domain, the N-terminus of said second linker domain is attached the C-terminus of said lipid binding domain, the N-terminus of said pseudoligand is attached to the C-terminus of said second linker domain, and the N-terminus of said third linker is attached to the C-terminus of said pseudoligand.

4. The biosensor polypeptide of any one of claims 1-3, wherein said lipid binding domain is capable of binding phosphatidylinositol(3,4,5)trisphosphate (PIP₃).

5. The biosensor polypeptide of any one of claims 1-4, wherein said pseudoligand is capable of binding to said lipid binding domain.

6. The biosensor polypeptide of any one of claims 1-5, further comprising a donor and acceptor moiety pair comprising a donor moiety and an acceptor moiety.

7. The biosensor polypeptide of claim 6, wherein the N-terminus of said first linker is attached to the C-terminus of said donor moiety and the N-terminus of said acceptor moiety is attached to the C-terminus of said third linker.

8. The biosensor polypeptide of claim 6 or claim 7, wherein said donor moiety and said acceptor moiety comprise a fluorescence resonance energy transfer (FRET) pair, a chemiluminescence resonance energy transfer (CRET) pair, or a bioluminescence resonance energy transfer (BRET) pair.

9. The biosensor polypeptide of any one of claims 6-8, wherein said donor moiety is a fluorophore donor and said acceptor moiety is a fluorophore acceptor.

10. The method of claim 9, wherein said fluorophore donor is selected from the group consisting of a cerulean fluorescent polypeptide, a cyan fluorescent polypeptide, a blue fluorescent polypeptide, a green fluorescent polypeptide, a yellow fluorescent polypeptide, a red fluorescent polypeptide, a mT-Sapphire fluorescent polypeptide, a mTagBFP fluorescent polypeptide, a mAmertrine fluorescent polypeptide, a mTurquoise2 fluorescent polypeptide, a CyPet fluorescent polypeptide, and a LSSmOrange fluorescent polypeptide.

11. The method of claim 9 or claim 10, wherein said fluorophore acceptor is selected from the group consisting of a citrine fluorescent polypeptide, a cpVenus (VE172) fluorescent polypeptide, a tdTomato fluorescent polypeptide, a sfGFP fluorescent polypeptide, a mVenus fluorescent polypeptide, an EYFP fluorescent polypeptide, a mNeonGreen fluorescent polypeptide, a sREACh1 fluorescent polypeptide, a YPet fluorescent polypeptide, a mKate2 fluorescent polypeptide, a green fluorescent polypeptide, a yellow fluorescent polypeptide, a red fluorescent polypeptide, an orange fluorescent polypeptide, and a far-red fluorescent polypeptide.

12. The biosensor polypeptide of any one of claims 6-8, wherein said donor moiety is a chemiluminescence donor and said acceptor moiety is a chemiluminescence acceptor.

13. The biosensor polypeptide of any one of claims 1-12, wherein said first linker comprises the amino acid sequence set forth in SEQ ID NO:10.

14. The biosensor polypeptide of any one of claims 1-13, wherein said second linker comprises the amino acid sequence set forth in SEQ ID NO:11.

15. The biosensor polypeptide of any one of claims 1-14, wherein said third linker comprises the amino acid sequence set forth in SEQ ID NO:12.

16. The biosensor polypeptide of any one of claims 1-15, further comprising a targeting sequence selected from the group consisting of a nucleus targeting sequence, a Golgi apparatus targeting sequence, a lysosome targeting sequence, a plasma membrane targeting sequence, an endosome targeting sequence, an endoplasmic reticulum targeting sequence, and a mitochondria targeting sequence.

17. The biosensor polypeptide of claim 16, wherein said targeting sequence comprises an amino acid sequence selected from the group consisting of a sequence set forth in any one of SEQ ID NOs:43-54.

18. A biosensor polypeptide comprising:
a first linker;
a lipid binding domain comprising the amino acid sequence set forth in SEQ ID NO:2;
a second linker;
a pseudoligand comprising the amino acid sequence set forth in SEQ ID NO:9; and
a third linker.

19. The biosensor polypeptide of claim 18, wherein said biosensor polypeptide comprises, in order from amino terminus (N-terminus) to carboxy terminus (C-terminus):
said first linker;
said lipid binding domain comprising the amino acid sequence set forth in SEQ ID NO:2;
said second linker;
said pseudoligand comprising the amino acid sequence set forth in SEQ ID NO:9; and
said third linker.

20. The biosensor polypeptide of claim 18 or claim 19, wherein the N-terminus of said lipid binding domain is attached to the C-terminus of said first linker domain, the N-terminus of said second linker domain is attached the C-terminus of said lipid binding domain, the N-terminus of said pseudoligand is attached to the C-terminus of said second linker domain, and the N-terminus of said third linker is attached to the C-terminus of said pseudoligand.

21. The biosensor polypeptide of any one of claims 18-20, wherein said lipid binding domain is capable of binding phosphatidylinositol(3,4)bisphosphate (PI(3,4)P₂).

22. The biosensor polypeptide of any one of claims 18-21, wherein said pseudoligand is capable of binding to said lipid binding domain.

23. The biosensor polypeptide of any one of claims 18-22, further comprising a donor and acceptor moiety pair comprising a donor moiety and an acceptor moiety.

24. The biosensor polypeptide of claim 23, wherein the N-terminus of said first linker is attached to the C-terminus of said donor moiety and the N-terminus of said acceptor moiety is attached to the C-terminus of said third linker.

25. The biosensor polypeptide of claim 23 or claim 24, wherein said donor moiety and said acceptor moiety comprise a fluorescence resonance energy transfer (FRET) pair, a chemiluminescence resonance energy transfer (CRET) pair, or a bioluminescence resonance energy transfer (BRET) pair.

26. The biosensor polypeptide of any one of claims 23-25, wherein said donor moiety is a fluorophore donor and said acceptor moiety is a fluorophore acceptor.

27. The method of claim 26, wherein said fluorophore donor is selected from the group consisting of a cerulean fluorescent polypeptide, a cyan fluorescent polypeptide, a blue fluorescent polypeptide, a green fluorescent polypeptide, a yellow fluorescent polypeptide, a red fluorescent polypeptide, a mT-Sapphire fluorescent polypeptide, a mTagBFP fluorescent polypeptide, a mAmertrine fluorescent polypeptide, a mTurquoise2 fluorescent polypeptide, a CyPet fluorescent polypeptide, and a LSSmOrange fluorescent polypeptide.

28. The method of claim 26 or claim 27, wherein said fluorophore acceptor is selected from the group consisting of a citrine fluorescent polypeptide, a cpVenus (VE172) fluorescent polypeptide, a tdTomato fluorescent polypeptide, a sfGFP fluorescent polypeptide, a mVenus fluorescent polypeptide, an EYFP fluorescent polypeptide, a mNeonGreen fluorescent polypeptide, a sREACh1 fluorescent polypeptide, a YPet fluorescent polypeptide, a mKate2 fluorescent polypeptide, a green fluorescent polypeptide, a yellow fluorescent polypeptide, a red fluorescent polypeptide, an orange fluorescent polypeptide, and a far-red fluorescent polypeptide.

29. The biosensor polypeptide of any one of claims 23-25, wherein said donor moiety is a chemiluminescence donor and said acceptor moiety is a chemiluminescence acceptor.

30. The biosensor polypeptide of any one of claims 18-29, wherein said first linker comprises the amino acid sequence set forth in SEQ ID NO:10.

31. The biosensor polypeptide of any one of claims 18-30, wherein said second linker comprises the amino acid sequence set forth in SEQ ID NO:11.

32. The biosensor polypeptide of any one of claims 18-31, wherein said third linker comprises the amino acid sequence set forth in SEQ ID NO:12.

33. The biosensor polypeptide of any one of claims 18-32, further comprising a targeting sequence selected from the group consisting of a nucleus targeting sequence, a Golgi apparatus targeting sequence, a lysosome targeting sequence, a plasma membrane targeting sequence, an endosome targeting sequence, an endoplasmic reticulum targeting sequence, and a mitochondria targeting sequence.

34. The biosensor polypeptide of claim 33, wherein said targeting sequence comprises an amino acid sequence selected from the group consisting of a sequence set forth in any one of SEQ ID NOs:43-54.

35. A nucleic acid comprising a nucleic acid sequence encoding a biosensor polypeptide of any one of claims 1-34.

36. A vector comprising the nucleic acid of claim 35.

37. The vector of claim 36, wherein said vector is an expression plasmid.

38. The vector of claim 36, wherein said vector is a viral vector.

39. The vector of claim 38, wherein said viral vector is selected from the group consisting of a vector based on an adenovirus, a vector based on an adeno-associated virus (AAV), a vector based on a retrovirus, a vector based on a lentivirus, and a vector based on a baculovirus.

40. A method for detecting the cellular localization of a polypeptide in a cell, wherein said method comprises:
contacting said cell with a biosensor polypeptide of any one of claims 6-17 and 23-34, a nucleic acid of claim 35, or a vector of any one of claims 36-39; and
detecting a signal from said donor and acceptor moiety pair.

41. The method of claim 40, comprising detecting a change in said signal upon binding of a lipid to said lipid binding domain.

42. The method of claim 40 or claim 41, wherein said signal from said donor and acceptor moiety pair comprises the acceptor moiety emission to donor moiety emission ratio.

43. A method for detecting phosphoinositide 3-kinase (PI3K) pathway signaling in a cell, wherein said method comprises:
contacting said cell with a biosensor polypeptide of any one of claims 6-17 and 23-34, a nucleic acid of claim 35, or a vector of any one of claims 36-39; and
detecting a signal from said donor and acceptor moiety pair.

44. The method of claim 43, comprising detecting a change in said signal upon binding of a lipid to said lipid binding domain.

45. The method of claim 43 or claim 44, wherein said signal from said donor and acceptor moiety pair comprises the acceptor moiety emission to donor moiety emission ratio.


SEQUENCE LISTING (59 sequences)

SEQ ID NO: 1 - protein, 122 aa, synthetic construct
TFFNPDREGWLLKLGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASISRD

SEQ ID NO: 2 - protein, 122 aa, synthetic construct
FTPKPPQDSAVIKAGYCVKQGAVMKNWKRRYFQLDENTIGYFKSELEKEPLRVIPLKEVHKVQECKQSDIMMRDNLFEIVTTSRTFYVQADSPEEMHSWIKAVSGAIVAQRGPGRSASSEHP

SEQ ID NO: 3 - protein, 160 aa, synthetic construct
NEPFKIPEDDGNDLTHTFFNPDREGWLLKLGGGRVKTWKRRWFILTDNCLYYFEYTTDKEPRGIIPLENLSIREVDDPRKPNCFELYIPNNKGQLIKACKTEADGRVVEGNHMVYRISAPTQEEKDEWIKSIQAAVSVDPFYEMLAARKKRISVKKKQEQ

SEQ ID NO: 4 - protein, 165 aa, synthetic construct
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGC

SEQ ID NO: 5 - protein, 117 aa, synthetic construct
PDREGWLLKLGGGRVKTWKRRWFILTDNCLYYFEYTTDKEPRGIIPLENLSIREVEDSKKPNCFELYIPDNKDQVIKACKTEADGRVVEGNHTVYRISAPTPEEKEEWIKCIKAAIS

SEQ ID NO: 6 - protein, 100 aa, synthetic construct
LIKSGYCVKQGNVRKSWKRRFFALDDFTICYFKCEQDREPLRTIFLKDVLKTHECLVKSGDLLMRDNLFEIITSSRTFYVQADSPEDMHSWIKEIGAAVQ

SEQ ID NO: 7 - protein, 164 aa, synthetic construct
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKV

SEQ ID NO: 8 - protein, 96 aa, synthetic construct
LGTKEGYLTKQGGLVKTWKTRWFTLHRNELKYFKDQMSPEPIRILDLTECSAVQFDYSQERVNCFCLVFPFRTFYLCAKTGVEADEWIKILRWKLS

SEQ ID NO: 9 - protein, 14 aa, synthetic construct
VAEEEDDEEEDEDD

SEQ ID NO: 10 - skipped

SEQ ID NO: 11 - protein, 5 aa, synthetic construct
SAGGS

SEQ ID NO: 12 - protein, 5 aa, synthetic construct
GGSEL

SEQ ID NO: 13 - skipped

SEQ ID NO: 14 - protein, 239 aa, synthetic construct
MVSKGEELFTGVVPILVELDGDVNGHRFSVSGEGEGDATYGKLTLKFICTTGKLPVPWPTLVTTLTWGVQCFARYPDHMKQHDFFKSAMPEGYVQERTIFFKDDGNYKTRAEVKFEGDTLVNRIELKGIDFKEDGNILGHKLEYNAISDNVYITADKQKNGIKAHFKIRHNIEDGSVQLADHYQQNTPIGDGPVLLPDNHYLSTQSALSKDPNEKRDHMVLLEFVTAAGITLGMDELYK

SEQ ID NO: 15 - protein, 239 aa, synthetic construct
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNAIHGNVYITADKQKNGIKANFGLNCNIEDGSVQLADHYQQNTPIGDGPVLLPDNHYLSTQSKLSKDPNEKRDHMVLLEFVTAAGITLGMDELYK

SEQ ID NO: 16 - protein, 239 aa, synthetic construct
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 17 - protein, 238 aa, synthetic construct
MSKGEELFTGVVPILVELDGDVNGHKFSVSGEGEGDATYGKLTLKFICTTGKLPVPWPTLVTTFSHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 18 - protein, 238 aa, synthetic construct
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLGFVTAAGITHGMDELYK

SEQ ID NO: 19 - protein, 239 aa, synthetic construct
MVSKGEELFTGVVPILVELDGDVNGHKFSVRGEGEGDATNGKLTLKLISTTGKLPVPWPTLVTTLGYGLMVFARYPDHMKQHDFFKSAMPEGYVQERTISFEDDGYYKTRAEVKFEGDTLVNRIVLKGIDFKEDGNILGHKLEYNFNPHNVYITADKQKNGIKANFKIRHNVEDGGVQLADHYQQNTPIGDGPVLMPDNHYLSYQSKLSKDPNEKRDHMVLKERVTAAGITHDMNELYK

SEQ ID NO: 20 - protein, 225 aa, synthetic construct
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 21 - protein, 239 aa, synthetic construct
MVSKGEELFTGVVPILVELDGDVNGHKFSVSGEGEGDATYGKLTLKFICTTGKLPVPWPTLVTTFSYGVMVFARYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGGVQLADHYQQNTPIGDGPVLLPDNHYLSIQSALSKDPNEKRDHMVLLEFVTAAGITLGMDELYK

SEQ ID NO: 22 - protein, 233 aa, synthetic construct
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 23 - protein, 239 aa, synthetic construct
MVSKGEELFTGVVPILVELDGDVNGHKFSVRGEGEGDATNGKLTLKFICTSGKLPVPWPTLVTTLSYGVQCFARYPDHMKQHDFFKSAMPEGYVQERTISFKDDGSYRTRAEVKFEGDTLVNRIELKGIDFKEDGNILGHKLEYNMNVWDAYITADKQKNGIKANFKIEHNVEDGGVQLADAYQQNTPIGDGSVLLPDNHYLSFQSKLFKDPNEQRDHMVLLEFVTAAGITPGMDELYK

SEQ ID NO: 24 - protein, 239 aa, synthetic construct
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 25 - protein, 239 aa, synthetic construct
MVSKGEELFGGIVPILVELEGDVNGHKFSVSGEGEGDATYGKLTLKFICTTGKLPVPWPTLVTTLTWGVQCFSRYPDHMKQHDFFKSVMPEGYVQERTIFFKDDGNYKTRAEVKFEGDTLVNRIELKGIDFKEDGNILGHKLEYNYISHNVYITADKQKNGIKANFKARHNITDGSVQLADHYQQNTPIGDGPVILPDNHYLSTQSALSKDPNEKRDHMVLLEFVTAAGITHGMDELYK

SEQ ID NO: 26 - protein, 236 aa, synthetic construct
MVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYK

SEQ ID NO: 27 - protein, 245 aa, synthetic construct
MGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 28 - protein, 239 aa, synthetic construct
MVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 29 - protein, 476 aa, synthetic construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 30 - protein, 238 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 31 - protein, 239 aa, synthetic construct
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 32 - protein, 239 aa, synthetic construct
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 33 - protein, 236 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 34 - protein, 239 aa, synthetic construct
MVSKGEELFTGVVPILVELDGDVNGHKFSVSGEGEGDATYGKLTLKLICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYAWPVVNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 35 - protein, 239 aa, synthetic construct
MVSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALFKDPNEKRDHMVLLEFLTAAGITEGMNELYK

SEQ ID NO: 36 - protein, 231 aa, synthetic construct
MSELIKENMHMKLYMEGTVNNHHFKCTSEGEGKPYEGTQTMRIKVVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTEMLYPADGGLEGRSDMALKLVGGGHLICNLKTTYRSKKPAKNLKMPGVYYVDRRLERIKEADKETYVEQHEVAVARYCDLPSKLGHK

SEQ ID NO: 37 - protein, 238 aa, synthetic construct; identical to SEQ ID NO: 18

SEQ ID NO: 38 - protein, 239 aa, synthetic construct; identical to SEQ ID NO: 19

SEQ ID NO: 39 - protein, 222 aa, synthetic construct
MNLSKNVSVSVYMKGNVNNHEFEYDGEGGGDPYTGKYSMKMTLRGQNCLPFSYDIITTAFQYGFRVFTKYPEGIVDYFKDSLPDAFQWNRRIVFEDGGVLNMSSDITYKDNVLHGDVWAVGVNFPPNGPVMKNEIVMEEPTEETFTPKNGVLVGFCPKAYLLKDGSYYYGNMTTFYRSKKSGQAPPGYHFVKHRLVKTNVGHGFKTVEQTEYATAHVSDLPK

SEQ ID NO: 40 - protein, 225 aa, synthetic construct; identical to SEQ ID NO: 20

SEQ ID NO: 41 - protein, 313 aa, synthetic construct
MVAGHASGSPDFGTADPSDCEREEIHLAGSIQPHGTLLVVSEPDHRIIQASANAAEFLNLGSVLGVPLAEIDGDLLIKILPHLDPTAEGMPVAVRCRIGNPSTEYDGLMHRPPEGGLIIELERAGPPIDLSGTLAPALERIRTAGSLRALCDDTALLFQQCTGYDRVMVYRFDEQGHGEVYSEIHVTGLESYFGNRYPSSLVPQMARRLYERQRVRVLVDVSYQPVPLEPRLSPLTGRDLDMSGCFLRSMSPTHLQFLKNMGVRATLVVSLVVGGKLWGLVICHHYLPRFIHFELRAICELLAEAIATRITAL

SEQ ID NO: 42 - protein, 133 aa, synthetic construct
MAKTSEQRVNIATLLTENKKKIVDKASQDLWRRHPDLIAPGGIAFSQRDRALCLRDYGWFLHLITFCLLAGDKGPIESIGLISIREMYNSLGVPVPAMMESIRCLKEASLSLLDEEDANETAPYFDYIIKAMS

SEQ ID NO: 43 - protein, 417 aa, synthetic construct
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI

SEQ ID NO: 44 - skipped

SEQ ID NO: 45 - protein, 13 aa, synthetic construct
KKKKKSKTKCVIM

SEQ ID NO: 46 - protein, 216 aa, synthetic construct
RSTSRSTARPNGQPQASKICQFKLVLLGESAVGKSSLVLRFVKGQFHEYQESTIGAAFLTQSVCLDDTTVKFEIWDTAGQERYHSLAPMYYRGAQAAIVVYDITNQETFARAKTWVKELQRQASPSIVIALAGNKADLANKRMVEYEEAQAYADDNSLLFMETSAKTAMNVNDLFLAIAKKLPKSEPQNLGGAAGRSRGVDLHEQSQQNKSQCCSN

SEQ ID NO: 47 - protein, 155 aa, synthetic construct
MFAAERAPDWVDAEECHRCRVQFGVVTRKHHCRACGQIFCGKCSSKYSTIPKFGIEKEVRVCEPCYEQLNKKAQGQGSESDAMFAAERAPDWVDAEECHRCRVQFGVVTRKHHCRACGQIFCGKCSSKYSTIPKFGIEKEVRVCEPCYEQLNKKA

SEQ ID NO: 48 - protein, 27 aa, synthetic construct
MDPVVVLGLCLSCLLLLSLWKQSYGGG

SEQ ID NO: 49 - protein, 30 aa, synthetic construct
MAIQLRSLFPLALPGMLALLGWWWFFSRKK

SEQ ID NO: 50 - protein, 129 aa, synthetic construct
MSGRGKQGGKARAKAKSRSSRAGLQFPVGRVHRLLRKGNYAERVGAGAPVYMAAVLEYLTAEILELAGNAARDNKKTRIIPRHLQLAIRNDEELNKLLGKVTIAQGGVLPNIQAVLLPKKTESHKAKSK

SEQ ID NO: 51 - protein, 7 aa, synthetic construct
PKKKRKV

SEQ ID NO: 52 - protein, 9 aa, synthetic construct
LPPLERLTL

SEQ ID NO: 53 - protein, 123 aa, synthetic construct
FSEAQQQLCNTRQEVNELRKLLEEERDQRVAAENALSVAEEQIRRLEHSEWDSSRTPIIGSCGTQEQALLIDLTSNSCRRTRSGVGWKRVLRSLCHSRTRVPLLAAIYFLMIHVLLILCFTGH

SEQ ID NO: 54 - protein, 33 aa, synthetic construct
MAIQLRSLFPLALPGMLALLGWWWFFSRKKADP

SEQ ID NO: 55 - protein, 633 aa, synthetic construct
MVSKGEELFTGVVPILVELDGDVNGHRFSVSGEGEGDATYGKLTLKFICTTGKLPVPWPTLVTTLTWGVQCFARYPDHMKQHDFFKSAMPEGYVQERTIFFKDDGNYKTRAEVKFEGDTLVNRIELKGIDFKEDGNILGHKLEYNAISDNVYITADKQKNGIKAHFKIRHNIEDGSVQLADHYQQNTPIGDGPVLLPDNHYLSTQSALSKDPNEKRDHMVLLEFVTAAGITLGMDELYKRMHTFFNPDREGWLLKLGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDSAGGSVAEEEDDEEEDEDDGGSELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 56 - protein, 633 aa, synthetic construct
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNAIHGNVYITADKQKNGIKANFGLNCNIEDGSVQLADHYQQNTPIGDGPVLLPDNHYLSTQSKLSKDPNEKRDHMVLLEFVTAAGITLGMDELYKRMHFTPKPPQDSAVIKAGYCVKQGAVMKNWKRRYFQLDENTIGYFKSELEKEPLRVIPLKEVHKVQECKQSDIMMRDNLFEIVTTSRTFYVQADSPEEMHSWIKAVSGAIVAQRGPGRSASSEHPSAGGSVAEEEDDEEEDEDDGGSELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 57 - protein, 20 aa, synthetic construct
GGSVAEEEDDEEEDEDDGGS

SEQ ID NO: 58 - protein, 4 aa, synthetic construct
FYVE

SEQ ID NO: 59 - protein, 627 aa, synthetic construct
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNAIHGNVYITADKQKNGIKANFGLNCNIEDGSVQLADHYQQNTPIGDGPVLLPDNHYLSTQSKLSKDPNEKRDHMVLLEFVTAAGITLGMDELYKRMHFTPKPPQDSAVIKAGYCVKQGAVMKNWKRRYFQLDENTIGYFKSELEKEPLRVIPLKEVHKVQECKQSDIMMRDNLFEIVTTSRTFYVQADSPEEMHSWIKAVSGAIVAQRGPGRSASSEHPSAGGSVAEEEDDEEEDEDDGGSELMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK